SEQUENCE LISTING (59 sequences)

SEQ ID NO: 1 - DNA, 120 bp, Homo sapiens
ctgtccccggcataggtccatctctgcagaagccatttcaggagtacctggaggctcaacggcagaagcttcaccacaaaagcgaaatgggcacaccacaggtaagactttaatccggtt

SEQ ID NO: 2 - DNA, 228 bp, Homo sapiens
cgcctgtagggttgctgggaacacatgggcagggcgcccacctacacggggctgctgggcacccaagcactcagacggcagccctgccccttcccagtccgcctgcctgccttcgtctcctgagtgacccaccagcgtggggcagggcgtcgggccggctccctgtctcctccgggcaccagctcaggccggggcaggctgactcatgcgtaggagagtgggcagggc

SEQ ID NO: 3 - DNA, 228 bp, Homo sapiens; identical to SEQ ID NO: 2

SEQ ID NO: 4 - DNA, 120 bp, Homo sapiens; identical to SEQ ID NO: 1

SEQ ID NO: 5 - DNA, 120 bp, Homo sapiens
cggttccaagctgagcctaactggggaaatccagacggacatttccccaggaaacgggctgccttcccctcctggatctcctccatgacagggactctgaagagctgtgccaactcaggc

SEQ ID NO: 6 - DNA, 120 bp, Homo sapiens; identical to SEQ ID NO: 5

SEQ ID NO: 7 - DNA, 228 bp, Homo sapiens; identical to SEQ ID NO: 2

SEQ ID NO: 8 - DNA, 228 bp, Homo sapiens; identical to SEQ ID NO: 2

SEQ ID NO: 9 - DNA, 120 bp, Homo sapiens
gccgtaacctacacggagcacgccaagcgtaagacagtcactgcaatggatgttgtctacgcgctcaagcgccagggacgcactctgtacggctttggtggctgagcctcaccccggctt

SEQ ID NO: 10 - DNA, 120 bp, Homo sapiens; identical to SEQ ID NO: 9

SEQ ID NO: 11 - DNA, 431 bp, Homo sapiens
cggctgctagaggggagggaggggatctcgtgcccccgatctggcaccggggtgggcagggcatatggacggcagccattggcgaggtgcccacaccaggccctggcttcgggcccagcatgagcctgggccggcggtgggtaagctctctatccctctctgccctataaaaatccctggcagagcctccagtccatgcccgcaccgcctccgcgtcctcccgggctccccgtggaggggcaccaatttgtcctcgcctgcgcctgctcgggccagatggtgggtatttccaggccacaaagtcctctgacctttgaacagttgccgccgaatttcaataatgaaagggccttttttgaatatgtacaaatgagacgttatatttccatacattttatttccagcctcatctgcgaatctaatattgaccc

SEQ ID NO: 12 - DNA, 431 bp, Homo sapiens; identical to SEQ ID NO: 11

SEQ ID NO: 13 - DNA, 120 bp, Homo sapiens
gaaatagaaacttaagagaaatacacacttctgagaaactgaaacgacaggggaaaggaggtctcactgagcaccgtcccagcatccggacaccacagcggcccttcgctccacgcagaa

SEQ ID NO: 14 - DNA, 120 bp, Homo sapiens; identical to SEQ ID NO: 9

SEQ ID NO: 15 - DNA, 120 bp, Homo sapiens; identical to SEQ ID NO: 9

SEQ ID NO: 16 - DNA, 120 bp, Homo sapiens; identical to SEQ ID NO: 9

SEQ ID NO: 17 - DNA, 228 bp, Homo sapiens; identical to SEQ ID NO: 2

SEQ ID NO: 18 - DNA, 228 bp, Homo sapiens; identical to SEQ ID NO: 2

SEQ ID NO: 19 - DNA, 195 bp, Homo sapiens
gcttggccggatgtggggctctagcggggacctctttccctgagaacatctgggccatgcgttcgtgtctcccggcacccggccctgcccttgagaccaacaccatcctgattcttgatactttccgtgaaatgcgttggctcactggtttggtttttgtcctctctggaaactgttcagatgctctctttgttc

SEQ ID NO: 20 - DNA, 195 bp, Homo sapiens; identical to SEQ ID NO: 19

SEQ ID NO: 21 - DNA, 120 bp, Homo sapiens
ctgagtgtgggggctgtgggcccgcttggccagttgaggcagcagaatgcacctgcccccgaggtgagacctctgtcctccccacctgagtgcgggggctgtgggcccgcttggccggtt

SEQ ID NO: 22 - DNA, 120 bp, Homo sapiens; identical to SEQ ID NO: 13

SEQ ID NO: 23 - DNA, 120 bp, Homo sapiens; identical to SEQ ID NO: 13

SEQ ID NO: 24 - DNA, 120 bp, Homo sapiens; identical to SEQ ID NO: 13

SEQ ID NO: 25 - DNA, 120 bp, Homo sapiens; identical to SEQ ID NO: 13

SEQ ID NO: 26 - DNA, 120 bp, Homo sapiens
ggtcagggctgctgtcttttgagtcagccctggaaggcagaggtgcctgtggccacttgcgtcacttccccgcgtttgtggtgggaaggggagagatagatgggtctatctgctgaagaa

SEQ ID NO: 27 - DNA, 120 bp, Homo sapiens
ttgaggtgacaggcacgtgaaccgccctgatctgactatcccacatcatacacgggcgtcgaaacaccaccctgtattccagaaacaggtgcaattattgtgtgccaattaaaaaaagga

SEQ ID NO: 28 - DNA, 120 bp, Homo sapiens; identical to SEQ ID NO: 27

SEQ ID NO: 29 - DNA, 120 bp, Homo sapiens; identical to SEQ ID NO: 27

SEQ ID NO: 30 - DNA, 120 bp, Homo sapiens
aatggtggcagaggaatctctagctctttcagtcttagcttttcatattgtgaaactctcgcttgtacaattcttgggtcctttgtagagcttagcttagtttttctggtggcaaggcag

SEQ ID NO: 31 - DNA, 120 bp, Homo sapiens
ggtgctctaaaccctgcttcctgtccctgcgccccacaaggaggctgtgcctaacggtctgacccgtttgcacgccaggggcgaggggccggatgctgggtcctccccgctttggagggt

SEQ ID NO: 32 - DNA, 120 bp, Homo sapiens; identical to SEQ ID NO: 31

SEQ ID NO: 33 - DNA, 120 bp, Homo sapiens
ctcctagagccactcttcctggtgctggactaagaggtgcaggcttggagggtgcagggcggtccgcctctcagacgtagaggcccggcctcggatgaaggcggaagggagggcaccgcc

SEQ ID NO: 34 - DNA, 120 bp, Homo sapiens; identical to SEQ ID NO: 33

SEQ ID NO: 35 - DNA, 120 bp, Homo sapiens
gtgcactggggtcaccttagaccacaggaaatgtctggttaacacacgaagagatggaaacgctcgcagccacgccgcaaacggttagtcacgccccacagcctgcactcctcccagcgc

SEQ ID NO: 36 - DNA, 120 bp, Homo sapiens; identical to SEQ ID NO: 35

SEQ ID NO: 37 - DNA, 120 bp, Homo sapiens; identical to SEQ ID NO: 35

SEQ ID NO: 38 - DNA, 120 bp, Homo sapiens; identical to SEQ ID NO: 35

SEQ ID NO: 39 - DNA, 120 bp, Homo sapiens
gttggggaaggaatggaaccagggtttaacgaggttggcctcatccggtggttttcgcagcacgctacacaggcacagaagcagcttgcagtgagttgttaaaattgacgtggtgatagg

SEQ ID NO: 40 - DNA, 120 bp, Homo sapiens; identical to SEQ ID NO: 39

SEQ ID NO: 41 - DNA, 216 bp, Homo sapiens
catgccggggaggatggaggatccgtgcacgtccggggctgagcagcgctccagggagaggaacggtacctgcgcctcctgcaggaagctggcatattcctccgcccctgcgaacacagagcgccttcttcacaccccatctgacaacgcttgccgcccggacgatggacaaagctgctccaggcgcttgtaaacccactcatgcccttcctctct

SEQ ID NO: 42 - DNA, 216 bp, Homo sapiens; identical to SEQ ID NO: 41

SEQ ID NO: 43 - DNA, 120 bp, Homo sapiens; identical to SEQ ID NO: 33

SEQ ID NO: 44 - DNA, 120 bp, Homo sapiens; identical to SEQ ID NO: 33

SEQ ID NO: 45 - DNA, 120 bp, Homo sapiens
gcgagggccagggagatggagccaggtgcggaaagggttgcagcctcgcgggctgaggccggagggtgaaggcgccgctccttgaagtgaggaaaactccggtgaggaagaggctctttc

SEQ ID NO: 46 - DNA, 195 bp, Homo sapiens
cttcagtgcacgtggctgtttgtcccctcagagtaggaagcagctgttctaacttccgtcttcgcagaaggttgtcatctcctctgtggtgagggctctgcagagtcactgcttgtttgaccacattcagattgcggccagggctaaagtcacagcttacctcttcagctctctaccgaactgtattttccttga

SEQ ID NO: 47 - DNA, 195 bp, Homo sapiens; identical to SEQ ID NO: 46

SEQ ID NO: 48 - DNA, 120 bp, Homo sapiens
ccctggtattgtgcctgtttgggggaagaaaacgtcaataaaaattaattgatgagttggcagggcgggcggtgcgggttcgcggcgaggcgcagggtgtcatggcaaatgttacggctc

SEQ ID NO: 49 - DNA, 120 bp, Homo sapiens; identical to SEQ ID NO: 48

SEQ ID NO: 50 - DNA, 120 bp, Homo sapiens; identical to SEQ ID NO: 48

SEQ ID NO: 51 - DNA, 120 bp, Homo sapiens; identical to SEQ ID NO: 48

SEQ ID NO: 52 - DNA, 120 bp, Homo sapiens; identical to SEQ ID NO: 33

SEQ ID NO: 53 - DNA, 120 bp, Homo sapiens; identical to SEQ ID NO: 33

SEQ ID NO: 54 - DNA, 220 bp, Homo sapiens
tggtttcctgatgagcagacaaaattcggtcaggggccatggaaggagccgggagcgaccggaccagggcgaatctgctgtctgtgccggcggaaggccagtaagtgccgtgcagtcgtgaaaaaggaagtggttaaagtggttacggcaaagcggtggcgccggctgctcccgacactgcagcccgagagggagagaggaggactgaaggcagtcccag

SEQ ID NO: 55 - DNA, 220 bp, Homo sapiens; identical to SEQ ID NO: 54

SEQ ID NO: 56 - DNA, 120 bp, Homo sapiens
cctgtgagaggaagctgctgtgattcagagaagagacttcaagctgtgtgtgaccctggcgtccggttcctctcacaggctggagcttttcggaagtggcatgcaaagagtccaggtttg

SEQ ID NO: 57 - DNA, 120 bp, Homo sapiens; identical to SEQ ID NO: 56

SEQ ID NO: 58 - DNA, 33 bp, Artificial Sequence
  Synthetic Construct
  modified_base (2)..(32): c is 5-methylcytosine
acactctttccctacacgacgctcttccgatct

SEQ ID NO: 59 - DNA, 32 bp, Artificial Sequence
  Synthetic Construct
  modified_base (4)..(31): c is 5-methylcytosine
gatcggaagagcacacgtctgaactccagtca

(19) United States
(12) Patent Application Publication
(10) Pub. No.: US 2024/0209453 A1
(43) Pub. Date: Jun. 27, 2024

(54) LIVER CANCER METHYLATION AND PROTEIN MARKERS AND THEIR USES

(71) Applicants: Kenneth CHAHINE, Irvine, CA (US); Jianfeng XU, Irvine, CA (US); Maxime A. GALLANT, Irvine, CA (US); David J. TAGGART, West Lafayette, CA (US); Dhruvajyoti ROY, Irvine, CA (US); Helio Health Inc., Irvine, CA (US)

(72) Inventors: Kenneth CHAHINE, Park City, UT (US); Jianfeng XU, Irvine, CA (US); Maxime A. GALLANT, Irvine, CA (US); David J. TAGGART, West Lafayette, IN (US); Dhruvajyoti ROY, Irvine, CA (US)

(21) Appl. No.: 18/555,711

(22) PCT Filed: Apr. 21, 2022
(86) PCT No.: PCT/US2022/025826

§ 371 Date: Oct. 16, 2023

Related U.S. Application Data
(60) Provisional application No. 63/177,933, filed on Apr. 21, 2021.

(51) Int. Cl. C12Q 1/6886; C12Q 1/6806; C12Q 1/6827; C12Q 1/6874; G16H 50/20

(52) U.S. Cl. CPC C12Q 1/6886; C12Q 1/6806; C12Q 1/6827; C12Q 1/6874; G16H 50/20; C12Q 2600/106; C12Q 2600/118; C12Q 2600/154

(57) ABSTRACT

Disclosed herein, in some aspects, are methods for identifying a subject having liver cancer, in particular, hepatocellular carcinoma. Also provided herein, in certain aspects, are methods for generating a methylation profile of a biomarker and/or protein marker associated with liver cancer, and system, kits, and components thereof (such as probes) useful for the methodology described herein.

### CROSS-REFERENCE TO RELATED APPLICATION

This application claims the priority benefit of U.S. Provisional Application No. 63/177,933 filed on Apr. 21, 2021, entitled “LIVER CANCER METHYLATION AND PROTEIN MARKERS AND THEIR USES,” the contents of which are incorporated herein by reference in its entirety for all purposes.

### TECHNICAL FIELD OF THE INVENTION

The present invention relates generally to methods for the detection of liver cancer, in particular, hepatocellular carcinoma. More specifically, the present invention relates to methods for the detection and diagnosis of hepatocellular carcinoma through the quantitative and qualitative profiling of selected methylation and protein markers.

### BACKGROUND OF THE DISCLOSURE

Liver cancer, in particular the hepatocellular carcinoma (HCC) is the fifth most common neoplasm in the world and the leading cause of death among cirrhotic patients. Any focal liver lesion in a patient with cirrhosis is suggestive of HCC, and early detection may permit curative treatment in 30%-40% of patients (Bruix J, et al. J Hepatol. 2001; 35:421-430). α-fetoprotein (AFP) assay is the most frequent biologic screening test, but the diagnostic performance is poor. The radiologic modality most widely used for screening is ultrasonography, with a sensitivity around 45% for early detection of HCC (Tzartzeva K, et al. Gastroenterology 2018; 154:1706-18).

The threat of HCC is expected to continue to grow in the coming years (Llovet J M, et al., Liver Transpl. 2004 Feb. 10(2 Suppl 1):S115-20). Accordingly, there is a great need for early detection of HCC to improve the survival rate of these patients.

Reliable non-invasive screening methods with improved sensitivity and specificity are critical and urgently needed for the accurate detection of HCC, particularly in high-risk subjects who exhibit symptoms of cirrhosis in the presence or absence of chronic hepatitis.

In addition, the quality of the sequence analysis is, in part, a reflection of the quality of the starting material. It is vital that the preparation that is to be subjected to sequence analysis be of high quality, that is, relatively pure and free of contamination.

Thus, there is also a need for compositions and methods of rapid target enrichment or selection of nucleic acids for next generation sequencing (NGS) and downstream analysis.

### SUMMARY OF THE INVENTION

In some aspects, provided herein is a method of generating a biomarker profile from a sample obtained from an individual, wherein the biomarker profile comprises a methylation profile comprising data of one or more CpG sites from Table 11, the method comprising: (a) determining a methylation status for each of the one or more CpG sites of the methylation profile from a treated genomic DNA derived from the sample; and (b) generating the methylation profile based on the methylation status of the one or more CpG site of the methylation profile to generate the biomarker profile.

In some embodiments, the one or more CpG sites of the methylation profile comprises one or more CpG sites of one or more of the following genes: PSD4, EVL, RASSF5, MAP3K8, LAT2, HEXDC, MYO1G, CTTN, UBE4B, KIAA0930, LTA, C16orf54, LOC101928253, URI1, TNFAIP8L2 (SCNM1), FOXP4 (AS1), IFITM1, RPS6KA1, LINC01298, HIST1H4F, BDH1, MIR153-2, PFN3, LOC101929153, MIR1302-7, LOC100506585, DIRAS1, or MIR21.

In some embodiments, the one or more CpG sites of the methylation profile comprises one or more CpG sites of the following genes: PSD4, EVL, RASSF5, MAP3K8, LAT2, HEXDC, MYO1G, CTTN, UBE4B, KIAA0930, LTA, C16orf54, LOC101928253, URI1, TNFAIP8L2 (SCNM1), FOXP4 (AS1), IFITM1, RPS6KA1, LINC01298, HIST1H4F, BDH1, MIR153-2, PFN3, LOC101929153, MIR1302-7, LOC100506585, DIRAS1, and MIR21.

In some embodiments, the one or more CpG sites of the methylation profile comprises one or more of the following CpG sites: chr17:57915773-57915774, chr19:2723147-2723148, chr19:2723034-2723035, chr17:57915717-57915718, chr5:4629212-4629213, chr5:4629193-4629194, chr19:2723181-2723182, chr19:2723169-2723170, chr6:26240930-26240931, chr6:26240920-26240921, chr19:30562385-30562386, chr19:30562320-30562321, chr11:314074-314075, chr6:26240975-26240976, chr6:26240950-26240951, chr6:26240939-26240940, chr19:2723189-2723190, or chr19:2723184-2723185.

In some embodiments, the one or more CpG sites of the methylation profile comprises the following CpG sites: chr17:57915773-57915774, chr19:2723147-2723148, chr19:2723034-2723035, chr17:57915717-57915718, chr5:4629212-4629213, chr5:4629193-4629194, chr19:2723181-2723182, chr19:2723169-2723170, chr6:26240930-26240931, chr6:26240920-26240921, chr19:30562385-30562386, chr19:30562320-30562321, chr11:314074-314075, chr6:26240975-26240976, chr6:26240950-26240951, chr6:26240939-26240940, chr19:2723189-2723190, and chr19:2723184-2723185.

In some embodiments, the one or more CpG sites of the methylation profile comprises the following CpG sites: chr17:57915773-57915774, chr19:2723147-2723148, chr19:2723034-2723035, chr17:57915717-57915718, chr5:4629212-4629213, chr5:4629193-4629194, chr19:2723181-2723182, chr19:2723169-2723170, chr6:26240930-26240931, chr6:26240920-26240921, chr19:30562385-30562386, chr19:30562320-30562321, chr11:314074-314075, chr6:26240975-26240976, chr6:26240950-26240951, chr6:26240939-26240940, chr19:2723189-2723190, chr19:2723184-2723185, chr8:142852883-142852884, chr8:142852876-142852877, chr7:157563602-157563603, chr11:314113-314114, chr11:314106-314107, chr11:314098-314099, chr11:314086-314087, chr1:206753453-206753454, chr7:157319206-157319207, chr7:157319203-157319204, chr7:157319199-157319200, chr1:151129298-151129299, chr7:73641105-73641106, chr7:73641071-73641072, chr16:29757375-29757376, chr16:29757360-29757361, chr11:70211540-70211541, chr11:70211534-70211535, chr11:70211531-70211532, chr11:70211523-70211524, chr14:100532797-100532798, chr14:100532790-100532791, chr5:176829777-176829778, chr5:176829755-176829756, chr16:29757350-29757351, chr16:29757323-29757324, chr3:197283111-197283112, chr6:11976066-11976067, chr6:11976024-11976025, chr6:41528502-41528503, chr6:41528499-41528500, chr6:41528497-41528498, chr6:41528491-41528492, chr16:29757344-29757345, chr16:29757334-29757335, chr17:80358932-80358933, chr17:80358919-80358920, chr6:31527920-31527921, chr6:31527893-31527894, chr6:31527889-31527890, chr2:113931525-113931526, chr2:113931518-113931519, chr7:45018849-45018850, chr8:96193941-96193942, chr8:96193898-96193899, chr1:26872538-26872539, chr1:26872525-26872526, chr1:26872518-26872519, chr22:45631384-45631385, chr22:45631379-45631380, chr10:30818618-30818619, chr10:30818611-30818612, chr10:30818609-30818610, chr1:10134620-10134621, chr1:10134610-10134611, chr17:80358850-80358851, chr17:80358847-80358848, chr17:80358829-80358830, and chr17:80358819-80358820.

In some embodiments, the methylation status of each CpG site is based on a p-value, and wherein the 0-value of a CpG site is determined based on the proportion of instances of methylation at the CpG site divided by the sum of the instances of methylation at the CpG site plus the instances where the CpG site is not methylated.

In some embodiments, the methylation status is determined using sequencing information derived from the treated genomic DNA. In some embodiments, the sequencing information is obtained using a sequencing technique. In some embodiments, the sequencing technique is a next generation sequencing technique. In some embodiments, the sequencing technique is a whole-genome sequencing technique. In some embodiments, the sequencing technique is a targeted sequencing technique. In some embodiments, the sequence technique is capable of providing paired-end sequencing reads. In some embodiments, the sequencing technique is performed such that the sequencing depth is at least about 50×.

In some embodiments, the method further comprises performing the sequencing technique.

In some embodiments, the method further comprises obtaining the treated genomic DNA derived from the sample. In some embodiments, the obtaining the treated genomic DNA comprises subjecting DNA derived from the sample to processing that enables determination of a methylation status of a CpG. In some embodiments, the processing to obtain the treated genomic DNA comprises an enzyme-based technique for the conversion of unmethylated cytosines to enable the determination of the methylation status of a CpG site. In some embodiments, the enzyme-based technique is an EM-seq technique. In some embodiments, the processing to obtain the treated genomic DNA comprises a bisulfite-based technique.

In some embodiments, the detecting the methylation status for each of the one or more CpG sites is based on sequence reads obtained from the treated genomic DNA.

In some embodiments, the sequence reads used for the detecting the methylation status for each of the one or more CpG sites are pre-processed. In some embodiments, the sequence read pre-processing comprises removing low-quality reads. In some embodiments, the sequence read pre-processing comprises removing sequence adaptor sequences. In some embodiments, the sequence read pre-processing comprises removing M-bias. In some embodiments, the sequence read pre-processing comprises producing paired reads. In some embodiments, the sequence read pre-processing comprises removing sequence reads having a sequencing depth of less than 50×. In some embodiments, the sequence read pre-processing comprises mapping sequence reads to a reference genome. In some embodiments, the reference genome is a human reference genome.

In some embodiments, the biomarker profile further comprises a polypeptide profile. In some embodiments, the polypeptide profile comprises data of one or more of an alpha fetoprotein (AFP) level, a Lens culinaris agglutinin-reactive AFP (AFP-L3%) level, or a des-gamma-carboxyprothrombin (DCP) level obtained from the individual. In some embodiments, the polypeptide profile comprises data of the AFP level, AFP-L3%, and the DCP level. In some embodiments, the AFP level, AFP-L3%, and DCP level are based on respective serum concentrations measured from the individual. In some embodiments, the serum concentrations are derived from the sample obtained from the individual.

In some embodiments, the biomarker profile further comprises a demographic profile. In some embodiments, the demographic profile comprises the age of the individual. In some embodiments, the demographic profile comprises the sex of the individual.

In other aspects, provided herein is a method of generating a biomarker profile from a sample obtained from an individual, wherein the biomarker profile comprises: a methylation profile comprising data of one or more CpG sites from Table 11; a polypeptide profile comprising data of one or more of an AFP level, an AFP-L3%, or a DCP level; and a demographic profile comprising data of one or more of the age or sex of the individual, the method comprising: (a) determining, for the methylation profile, a methylation status for each of the one or more CpG sites of the methylation profile from a treated genomic DNA derived from the sample; (b) determining, for the polypeptide profile, one or more the AFP level, the AFP-L3%, or the DCP level from the sample; (c) determining, for the demographic profile, one or more of the age or sex of the individual; and (d) generating the biomarker profile based on the methylation profile, the polypeptide profile, and the demographic profile. In some embodiments, the methylation profile comprises data of all CpG sites from Table 11. In some embodiments, the polypeptide profile comprises the AFP level, the AFP-L3%, and the DCP level. In some embodiments, the demographic profile comprises the age and sex of the individual.

In some embodiments, the generating the biomarker profile comprises providing the methylation profile, the polypeptide profile, and/or the demographic profile to one or more machine learning classifiers to generate the biomarker profile. In some embodiments, the one or more machine learning classifiers comprises a random forest model. In some embodiments, the one or more machine learning classifiers comprises a grid-search technique. In some embodiments, the grid-search technique comprises optimizing the hyper parameters of the random forest model.

In some embodiments, the biomarker profile combines the methylation profile, the polypeptide profile, and/or the demographic profile using a decision tree model.

In some embodiments, at least one of the one or more machine learning classifiers is trained using a data derived from one or more individuals having known condition(s) and one or more associated methylation profiles, polypeptide profiles, or demographic profiles. In some embodiments, the known condition is whether the individual has a liver cancer or chronic liver disease.

In some embodiments, the sample is a liquid biopsy sample. In some embodiments, the sample is a blood sample. In some embodiments, the sample comprises cfDNA. In some embodiments, the sample is a cfDNA sample.

In some embodiments, the subject is suspected of having a liver cancer. In some embodiments, the liver cancer is hepatocellular carcinoma.

In other aspects, provided is a system for determining a biomarker profile from a sample obtained from an individual, wherein the biomarker profile comprises one or more of: a methylation profile comprising data of one or more CpG sites from Table 11; a polypeptide profile comprising data of one or more of an AFP level, an AFP-L3%, or a DCP level; or a demographic profile comprising data of one or more of the age or sex of the individual, the system comprising: one or more processors; and memory storing one or more programs, the one or more programs configured to be executed by the one or more processors, the one or more programs including instructions for: receiving sequencing information comprising sequence reads; determining one or more of the following: the methylation profile based on data of the one or more CpG sites from Table 11; the polypeptide profile based on data of the one or more of the AFP level, the AFP-L3%, or the DCP level; or the demographic profile based on data of the one or more of the age or sex of the individual, determining the biomarker profile based on one or more of the methylation profile, the polypeptide profile, or the demographic profile.

In some embodiments, the system further comprises one or more machine learning classifiers configured to determine the biomarker profile.

In other aspects, provided is a system for determining a biomarker profile from a sample obtained from an individual, wherein the biomarker profile comprises one or more of: a methylation profile comprising data of one or more CpG sites from Table 11; a polypeptide profile comprising data of one or more of an AFP level, an AFP-L3%, or a DCP level; or a demographic profile comprising data of one or more of the age or sex of the individual, the system comprising: one or more processors; and memory storing one or more programs, the one or more programs configured to be executed by the one or more processors, the one or more programs including instructions for: receiving data pertaining to one or more of the methylation profile, the polypeptide profile, and the demographic profile; applying one or more machine learning classifiers to the received data to determine the biomarker profile based on one or more of the methylation profile, the polypeptide profile, or the demographic profile. In some embodiments, the one or more machine learning classifiers comprises a random forest model. In some embodiments, the one or more machine learning classifiers comprises a grid-search technique. In some embodiments, the grid-search technique comprises optimizing the hyper parameters of the random forest model. In some embodiments, the biomarker profile combines the methylation profile, the polypeptide profile, and/or the demographic profile using a decision tree model. In some embodiments, at least one of the one or more machine learning classifiers is trained using a data derived from one or more individuals having known condition(s) and one or more associated methylation profiles, polypeptide profiles, or demographic profiles. In some embodiments, the known condition is whether the individual has a liver cancer or chronic liver disease.

In other aspects, provided herein is a kit for generating a biomarker profile from a sample from an individual, the kit comprising one or more probes, wherein each probe is suitable for detecting a methylation status of a CpG site in Table 11. In some embodiments, each probe hybridizes to at least a portion of the targeted region in Table 11. In some embodiments, the at least the portion is at least about 50 base pairs. In some embodiments, the at least the portion is about 120 base pairs. In some embodiments, the each probe is complementary to the target portion.

In some embodiments, each probe is about 50 to about 120 base pairs. In some embodiments, each probe is configured to determine the methylation status of one or more CpG sties from Table 11.

In some embodiments, the kit further comprises reagents to determine one or more of an AFP level, an AFP-L3%, or a DCP level from a sample from the individual.

In some embodiments, the kit further comprises instructions for determining the age and/or sex of the individual.

Provided herein are methods, composition and kits for identifying a subject as having liver cancer. Also provided herein are methods and kits for determining the prognosis of a subject having liver cancer. Further provided herein are methods and kits for determining the progression of liver cancer in a subject.

The subject methods may be employed to diagnose hepatocellular carcinoma, for example. In particular embodiments, the subject methods may be employed to differentiate between a subject having hepatocellular carcinoma and a subject having cirrhosis.

In certain embodiments, measuring the level of methylation in said biological sample at a CpG dinucleotide sequence in a genomic target. In certain embodiments, a method for detecting the presence and/or amount of methylated cytosine specific to liver cancer on a region containing CpG sequences in following genes and/or following genes comprising the following steps of (a) to (d): a) isolating the genomic DNA from the sample from a patient; (b) providing a reagent for chemical or enzymatical treatments to the genomic DNA in order to discriminate between methylated and unmethylated; (c) amplifying methylated cytosine-containing regions of a gene and/or multiple genes of the genomic DNA using PCR method; and (d) determining the presence and/or amount of methylated cytosine specific to liver cancer on a region containing CpG sequences in a gene and/or multiple genes in the specimen from said donor.

In certain embodiments, provided herein is a method of selecting a subject suspected of having liver cancer for treatment, the method comprising: (a) processing an extracted genomic DNA with a deaminating agent to generate a genomic DNA sample comprising deaminated nucleotides, wherein the extracted genomic DNA is obtained from a biological sample from the subject suspected of having liver cancer; (b) generating a methylation profile comprising one or more biomarkers selected from a chromosomal region having an annotation selected from the Table 1; capable of distinguishing liver cancer samples from benign liver diseases and healthy donor samples; (c) comparing the methylation profile of the one or more biomarkers with a control; (d) identifying the subject as having liver cancer if the methylation profile correlates to the control; and (e) administering an effective amount of a therapeutic agent to the subject if the subject is identified as having liver cancer.

In some embodiments, the methylation profile comprises one or more differentially methylated regions (DMRs).

In some embodiments, the method is related to screening for HCC in a sample obtained from a subject, the method comprising assaying a methylation state of a marker in a sample obtained from a subject; and identifying the subject as having HCC when the methylation state of the marker is different than a methylation state of the marker assayed in a subject that does not have HCC (e.g., a subject that does not have HCC) (e.g., a subject that does not have HCC but does have liver cirrhosis), wherein the marker comprises one or more bases in a DMR selected from UBE4B, TNFAIP8L2 (SCNM1), RASSF5, RPS6KA1, IFITM1, PPFIA1, SYT9, EVL, C16orf54, VMP1, OGFOD3, PSD4, KIAA0930, BDH1, F12, H4C6, LOC100287329, FOXP4 (AS1), PTPRN2, YZ2 (MYO1G), LAT2, MAP3K8, HEXDC, CTTN, LTA, LOC101928253, URI1, LINC01298, HIST1H4F, MIR153-2, PFN3, LOC101929153, MIR1302-7, LOC100506585, DIRAS1, and MIR21 as provided in Tables 1, 2, and 6. In some embodiments, the marker comprises one or more bases in a DMR selected from UBE4B, TNFAIP8L2 (SCNM1), RASSF5, RPS6KA1, IFITM1, EVL, C16orf54, PSD4, KIAA0930, BDH1, FOXP4 (AS1), YZ2 (MYO1G), LAT2, MAP3K8, HEXDC, CTTN, LTA, LOC101928253, URI1, LINC01298, HIST1H4F, MIR153-2, PFN3, LOC101929153, MIR1302-7, LOC100506585, DIRAS1, and MIR21 as provided in Tables 1, 2, and 6. In some embodiments, the marker comprises one or more bases in a DMR selected from UBE4B, TNFAIP8L2 (SCNM1), RASSF5, RPS6KA1, IFITM1, PPFIA1, SYT9, EVL, C16orf54, VMP1, OGFOD3, PSD4, KIAA0930, BDH1, F12, H4C6, LOC100287329, FOXP4 (AS1), PTPRN2, YZ2 (MYO1G), and LAT2 as provided in Tables 1, 2, and 6.

In some embodiments, the methylation profile comprises one or more bases in a DMR selected from ADCY10, FXYD6, BOLA2, CHA, RUNX1, B4GALT4, ACTR1, PRRT1, and SND1 as provided in Table 1.

In some embodiments, the methylation profile comprises one or more bases in a DMR selected from Table 6.

In some embodiments, the methylation profile comprises one or more bases in a DMR selected from one or more CpG sites captured by the probes listed in Table 2.

In some embodiments, the method determining a tumor DNA methylation profile from a tumor sample from the patient, the tumor DNA methylation profile comprising the methylation status of one or more CpG sites represented by the probes set forth in Table 2.

In some embodiments, the comparing further comprises generating a pair-wise methylation difference dataset comprising: (i) a first difference between the methylation profile of the treated genomic DNA with a methylation profile of a first normal sample; (ii) a second difference between a methylation profile of a second normal sample and a methylation profile of a third normal sample; and (iii) a third difference between a methylation profile of a first primary cancer sample and a methylation profile of a second primary cancer sample.

In some embodiments, the method comprises: a) fragmented genomic DNA, there by generating DNA fragments; b) performing end repair on the DNA fragments; c) ligating a single adapter forming a partial duplex to both ends of each DNA fragment.

In some embodiments, assaying the methylation state of the marker in the sample comprises determining the extent of methylation at a plurality of bases. Moreover, in some embodiments the methylation state of the marker comprises an increased methylation of the marker relative to a normal methylation state of the marker. In some embodiments, the methylation state of the marker comprises a decreased methylation of the marker relative to a normal methylation state of the marker. In some embodiments the methylation state of the marker comprises a different pattern of methylation of the marker relative to a normal methylation state of the marker.

In some embodiments, the comparing further comprises analyzing the pair-wise methylation difference dataset with a control by a machine learning method to generate the methylation profile.

In certain embodiments, the method comprises: a) obtaining hepatocellular carcinoma protein marker profile for a specimen obtained from the subject. b) comparing the protein marker profile to a control group.

In some embodiments, to evaluate whether a subject has HCC, the presence of one or more HCC protein markers in a sample is assessed to produce a profile, and that profile is compared to a control profile to evaluate HCC. The HCC protein marker profile may be employed to distinguish subjects having HCC from subjects having cirrhosis.

While a wide range of proteins may be employed as HCC protein markers, the HCC protein markers employed in many embodiments of the instant methods include proteins selected from the group consisting of: AFP, Lens culinaris agglutinin-reactive AFP (AFP-L3), des-gamma carboxy prothrombin (DCP), osteopontin, midkine (MDK), dikkopf-1 (DKK1), glypican-3 (GPC-3), alpha-1 fucosidase (AFU), and golgi protein-73 (GP-73).

In certain embodiments, the instant methods include: obtaining an HCC protein marker profile or multiple protein markers profile that include quantitative data for at least one protein marker selected from the group consisting of AFP, AFP-L3, and DCP, and comparing the profile with a control profile.

In certain embodiments, the method may further include evaluating AFP, DCP and AFP-L3 levels.

In some embodiments, the method comprising multiple biomarkers encompassing DNA methylation and multiple protein markers.

In some embodiments, the first primary cancer sample is a liver cancer sample.

In some embodiments, the second primary cancer sample is a non-liver cancer sample.

In some embodiments, the control comprises a set of methylation profiles, wherein each said methylation profile is generated from a biological sample obtained from a known cancer type.

In some embodiments, the known cancer type is liver cancer.

In some embodiments, the known cancer type is a relapsed or refractory liver cancer.

In some embodiments, the known cancer type is a metastatic liver cancer.

In some embodiments, the machine learning method utilizes an algorithm selected from one or more of the following: a principal component analysis, a logistic regression analysis, a nearest neighbor analysis, a support vector machine, and a neural network model.

In some embodiments, the method relates to characterizing samples, e.g., blood samples, any liquid biopsy specimen, for the presence or absence of, and/or the amounts of different species of nucleic acids that, for example, may be associated with a health status of a subject. The method further comprises determining a degree of confidence based on the level of each DNA methylation biomarker of the panel of DNA methylation markers; and determining a cutoff value; wherein when the degree of confidence is higher than the cutoff value, a diagnosis of cancer.

In some embodiments, the generating further comprises hybridizing each of the one or more biomarkers with a probe, and performing a DNA sequencing reaction to quantify the methylation of each of the one or more biomarkers.

In one aspect, the invention features a composition for nucleic acid hybridization.

In some embodiments, the composition further comprises a capture probe oligonucleotide, the capture probe oligonucleotide comprising a region that is complementary to a portion of a strand of genomic DNA. The capture probe is not limited to any particular configuration. In certain preferred embodiments, the capture probe oligonucleotide comprises a region that is complementary to a portion of bisulfite or enzymatic converted DNA or the complement thereof.

The kit further provides methods of characterizing samples. In some embodiments, the method comprises a) treating DNA from a sample with a bisulfite reagent or enzymes to produce bisulfite or enzymatic-converted DNA, and b) amplifying a region of the bisulfite-converted DNA.

The present invention pertains to methods of purifying a target molecule contained within a test sample. Typically, the target molecule in a test sample will be a nucleic acid molecule, in particular, single-stranded DNA or bisulfite or enzymatically converted DNA.

In some embodiments, the biological sample comprises a blood sample. In some embodiments, the biological sample comprises cell free DNA. In some embodiments, the biological sample comprises a tissue biopsy sample. In some embodiments, the biological sample comprises circulating tumor cells.

In some embodiments, the subject is a human.

In certain embodiments, provided herein is a method of generating a methylation profile of a biomarker in a subject in need thereof, comprising: (a) processing an extracted genomic DNA with a deaminating agent to generate a genomic DNA sample comprising deaminated nucleotides, wherein the extracted genomic DNA is obtained from a biological sample from the subject; (b) detecting a hybridization between the extracted genomic DNA and a probe, wherein the probe hybridizes to a biomarker selected from Table 1; and (c) generating a methylation profile based on the detected hybridization between the extracted genomic DNA and the probe.

In some embodiments, the generating further comprises generating a pair-wise methylation difference dataset comprising: (i) a first difference between the methylation profile of the treated genomic DNA with a methylation profile of a first normal sample; (ii) a second difference between a methylation profile of a second normal sample and a methylation profile of a third normal sample; and (iii) a third difference between a methylation profile of a first primary cancer sample and a methylation profile of a second primary cancer sample.

In some embodiments, the generating further comprises analyzing the pair-wise methylation difference dataset with a control by a machine learning method to generate the methylation profile.

In some embodiments, the first primary cancer sample is a liver cancer sample.

In some embodiments, the second primary cancer sample is a non-liver cancer sample.

In some embodiments, the control comprises a set of methylation profiles, wherein each said methylation profile is generated from a biological sample obtained from a known cancer type.

In some embodiments, the known cancer type is liver cancer. In some embodiments, the known cancer type is a relapsed or refractory liver cancer. In some embodiments, the known cancer type is a metastatic liver cancer. In some embodiments, the known cancer type is hepatocellular carcinoma (HCC), fibrolamellar HCC, cholangiocarcinoma, angiosarcoma, or hepatoblastoma.

In some embodiments, the machine learning method utilizes an algorithm selected from one or more of the following: a principal component analysis, a logistic regression analysis, a nearest neighbor analysis, a support vector machine, and a neural network model.

In some embodiments, the method further comprises performing a DNA sequencing reaction to quantify the methylation of each of the one or more biomarkers prior to generating the methylation profile.

In some embodiments, the biological sample comprises a blood sample. In some embodiments, the biological sample comprises cell free DNA. In some embodiments, the biological sample comprises a tissue biopsy sample. In some embodiments, the biological sample comprises circulating tumor cells. In some embodiments, the biological sample comprises analytes from liquid biopsy specimens.

In some embodiments, the subject is a human.

In certain embodiments, provided herein is a method, comprising: (a) determining the level of single protein or level of one or more proteins from a protein panel comprising AFP, AFP-L3 and DCP from the biological sample of the subjects, wherein the biological fluids (e.g., serum or plasma or both) are obtained from a biological sample from the subject having liver cancer (e.g., HCC) or having high risk for liver cancer; (b) generating a methylation profile comprising one or more biomarkers selected from the Table 6; (c) obtaining a methylation score based on the methylation profile of the one or more biomarkers; and (d) based on the methylation score, initiate a first treatment, decrease a dosage of a first therapeutic agent if the subject has experienced a remission, initiate a second treatment if the subject has experienced a relapse, or switch to a second therapeutic agent if the subject becomes refractory to the first therapeutic agent.

In some embodiments, liver cancer is metastatic liver cancer. In some embodiments, liver cancer is hepatocellular carcinoma (HCC), fibrolamellar HCC, cholangiocarcinoma, angiosarcoma, or hepatoblastoma.

In some embodiments, the generating further comprises hybridizing each of the one or more biomarkers with a probe, and performing a DNA sequencing reaction to quantify the methylation of each of the one or more biomarkers.

In some embodiments, the biological sample comprises a blood sample. In some embodiments, the biological sample comprises cell free DNA. In some embodiments, the biological sample comprises a tissue biopsy sample. In some embodiments, the biological sample comprises circulating tumor DNA.

In some embodiments, the subject is a human.

In certain embodiments, provided herein is a kit comprising a set of nucleic acid probes that hybridizes to biomarkers: one or more CpG sites from Table 6.

In some embodiments, the present invention relate generally to non-invasive methods, diagnostic tests, especially blood (including serum or plasma) tests that measure biomarkers (e.g.

DNA methylation or protein level), and computer-implemented machine learning methods, apparatuses, systems, and computer-readable media for assessing a likelihood that a patient has a disease, such as cancer, relative to a patient population or a cohort population to determine whether that patient should be followed up with additional, more invasive testing.

In one embodiment, techniques are provided for the use of artificial intelligence/machine learning systems that can incorporate and analyze structured and preferably also unstructured data to perform a risk analysis to determine a likelihood for having cancer, initially liver cancer, but also, other types of cancer, including pan-cancer testing (i.e. testing of multiple tumors from a single patient sample).

In some embodiments, the term “classification” refers to a procedure and/or algorithm in which individual items are placed into groups or classes based on quantitative information on one or more characteristics inherent in the items (referred to as traits, variables, characters, features, etc.) and based on a statistical model and/or a training set of previously labeled items. A “classification tree” is a decision tree that places categorical variables into classes.

In some embodiments, the term “diagnosis” is used herein to refer to the identification or classification of a molecular or pathological state, disease or condition. For example, “diagnosis” may refer to identification of a particular type of cancer, e.g., a liver cancer. “Diagnosis” may also refer to the classification of a particular type of cancer, e.g., by histology (e.g., a hepatocellular carcinoma), by DNA methylation level in a particular gene or genes and/or proteins), or combination of both.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates the identification of differentially “hypermethylated” or having “increased methylation” regions or site for discriminating HCC DNA samples from DNA derived from normal controls (e.g., non-HCC individuals with or without liver cirrhosis).

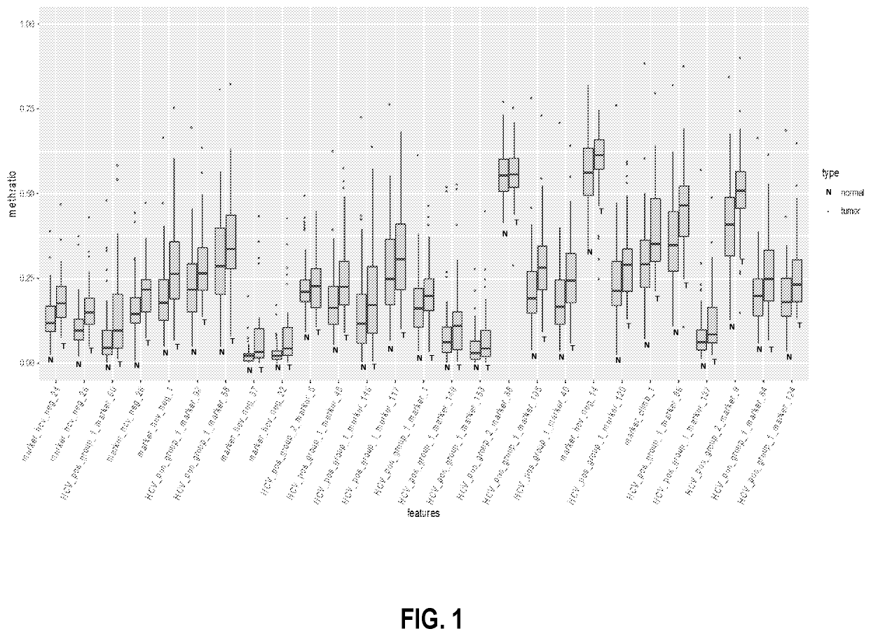

FIG. 2 shows the identification of differentially “hypomethylated” or having “decreased methylation” regions or site for discriminating HCC DNA samples from DNA derived from normal controls (e.g., non-HCC individuals with or without liver cirrhosis).

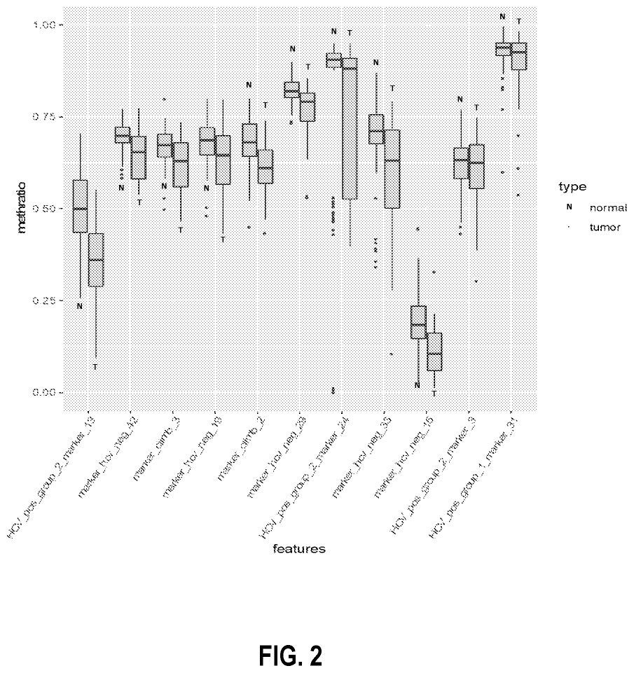

FIG. 3 shows the performance characteristics the pre-selected hypermethylated markers in an independent ethnic and geographic cohort in China (Benign vs. HCC).

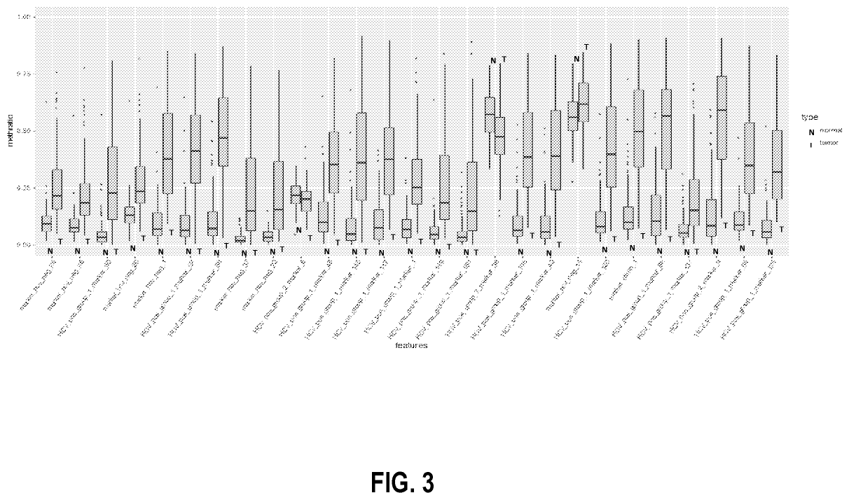

FIG. 4 shows the performance characteristics the pre-selected hypomethylated markers in an independent ethnic and geographic cohort in China (Benign vs. HCC).

FIG. 5 shows the performance characteristics the pre-selected hypermethylated markers in an independent ethnic and geographic cohort in China (Benign vs. HCC_Stage I).

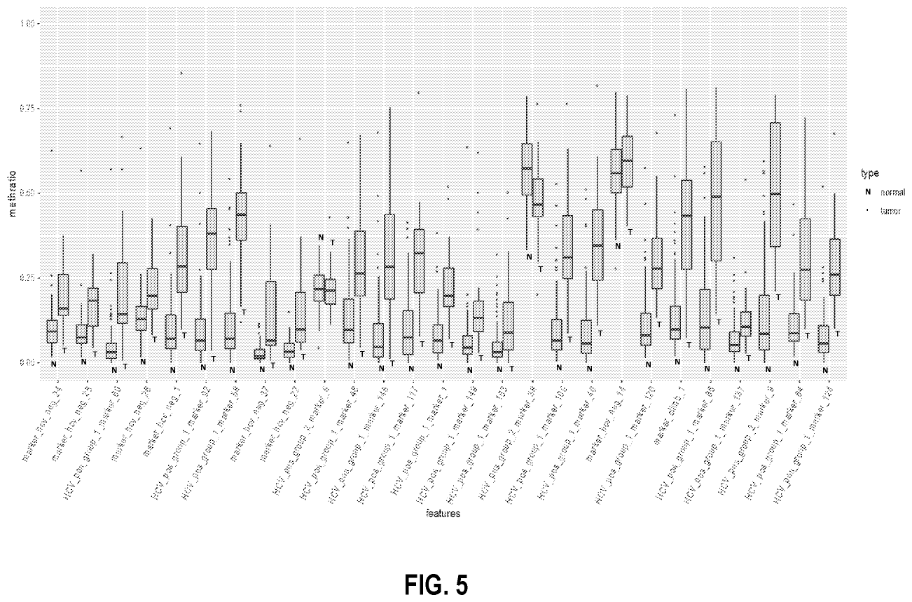

FIG. 6 shows the performance characteristics the pre-selected hypomethylated markers in an independent ethnic and geographic cohort in China (Benign vs. HCC_Stage I).

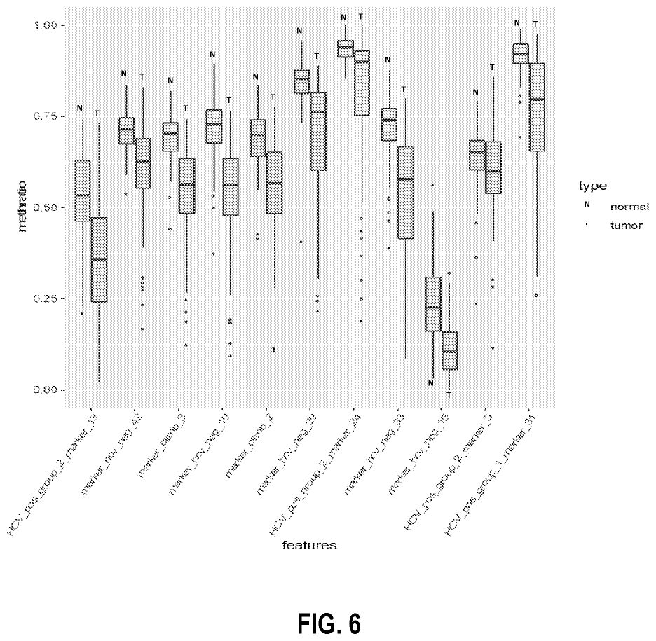

FIG. 7 illustrates the ROC curve and ROC-AUC scores from the cross-validation data set; showing the complementary of the three-protein score, the score predicted by the methylation markers, and the combined score.

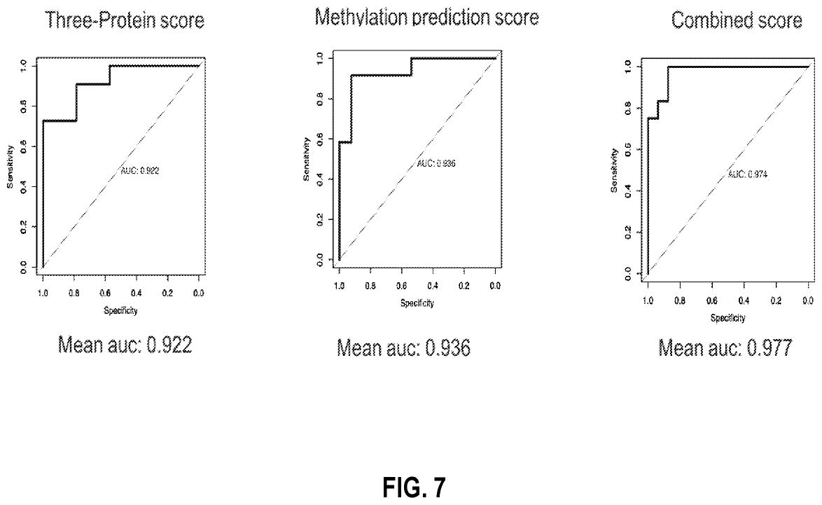

FIG. 8 shows the receiver operating characteristic (ROC) curves for the multi-analyte HCC test for hepatocellular carcinoma (HCC) compared to other biomarker-based tests [HCC (all stages) vs. Non-HCC]. The area under the curve (AUC) values for each ROC curve are included in parentheses. Performance of the multi-analyte HCC test (methylation+three-protein) was compared with (methylation+AFP), three-protein only, methylation alone, and AFP alone.

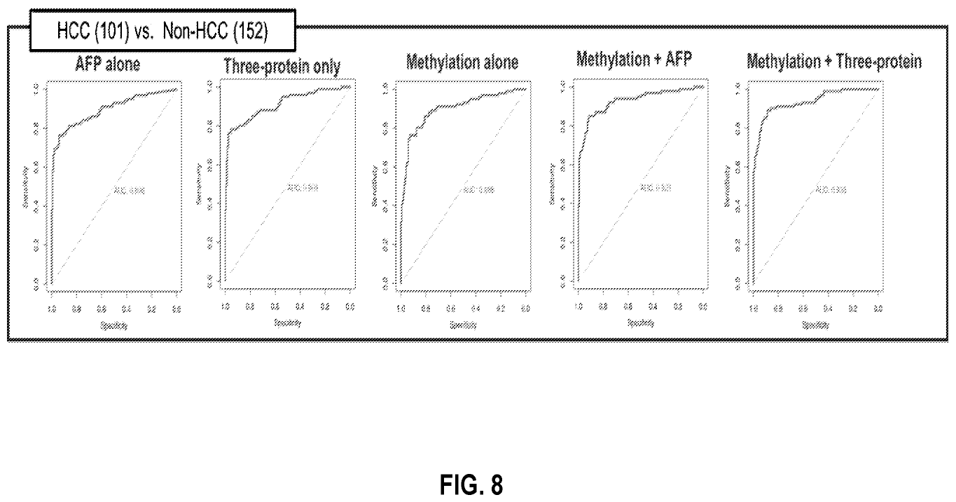

FIG. 9 shows the receiver operating characteristic (ROC) curves for the multi-analyte HCC test for hepatocellular carcinoma (HCC) compared to other biomarker-based tests [HCC (all stages) vs. Benign]. The area under the curve (AUC) values for each ROC curve are included in parentheses. Performance of the multi-analyte HCC test (methylation+three-protein) was compared with (methylation+AFP), three-protein only, methylation alone, and AFP alone.

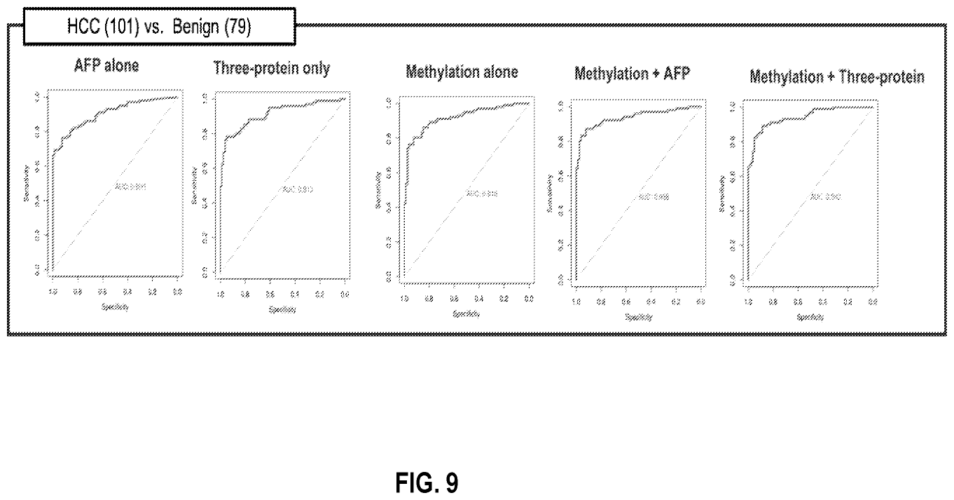

FIGS. 10A and 10B show receiver operating characteristic (ROC) curves for HCC blood tests. FIG. 10A shows ROC curves for analysis of all subjects diagnosed with HCC and control (benign liver disease) subjects. FIG. 10B shows subjects diagnosed with early-stage (AJCC stage I and II) HCC and control (benign liver disease) subjects. AUROC represents area under ROC.

### DETAILED DESCRIPTION OF THE DISCLOSURE

Cancer is characterized by an abnormal growth of a cell caused by one or more mutations or modifications of a gene leading to dysregulated balance of cell proliferation and cell death. DNA methylation silences expression of tumor suppression genes, and presents itself as one of the first neoplastic changes. Methylation patterns found in neoplastic tissue and plasma demonstrate homogeneity, and in some instances are utilized as a sensitive diagnostic marker. For example, cMethDNA assay has been shown in one study to be about 91% sensitive and about 96% specific when used to diagnose metastatic breast cancer. In another study, circulating tumor DNA (ctDNA) was about 87.2% sensitive and about 99.2% specific when it was used to identify KRAS gene mutation in a large cohort of patients with metastatic colon cancer (Bettegowda et al., Detection of Circulating Tumor DNA in Early- and Late-Stage Human Malignancies. Sci. Transl. Med, 6(224):ra24. 2014). The same study further demonstrated that ctDNA is detectable in >75% of patients with advanced pancreatic, ovarian, colorectal, bladder, gastroesophageal, breast, melanoma, hepatocellular, and head and neck cancers (Bettegowda et al).

Additional studies have demonstrated that CpG methylation pattern correlates with neoplastic progression. For example, in one study of breast cancer methylation patterns, P16 hypermethylation has been found to correlate with early stage breast cancer, while TIMP3 promoter hypermethylation has been correlated with late stage breast cancer. In addition, BMP6, CST6 and TRVIP3 promoter hypermethylation have been shown to associate with metastasis into lymph nodes in breast cancer.

In some embodiments, DNA methylation profiling provides higher clinical sensitivity and dynamic range compared to somatic mutation analysis for cancer detection. In other instances, altered DNA methylation signature has been shown to correlate with the prognosis of treatment response for certain cancers. For example, one study illustrated that in a group of patients with advanced rectal cancer, ten differentially methylated regions were used to predict patients' prognosis. Likewise, RASSF1A DNA methylation measurement in serum was used to predict a poor outcome in patients undergoing adjuvant therapy in breast cancer patients in a different study. In addition, SRBC gene hypermethylation was associated with poor outcome in patients with colorectal cancer treated with oxaliplatin in a different study. Another study has demonstrated that ESRI gene methylation correlate with clinical response in breast cancer patients receiving tamoxifen. Additionally, ARHI gene promoter hypermethylation was shown to be a predictor of long-term survival in breast cancer patients not treated with tamoxifen.

In some embodiments, disclosed herein are methods, compositions and kits of diagnosing liver cancer based on DNA methylation profiling. In some instances, provided herein are methods and kits of identifying a subject has having liver cancer based on the DNA methylation profiling. In some instances, also provided herein are methods and kits of determining the prognosis of a subject having liver cancer and determining the progression of liver cancer in a subject based on the DNA methylation profiling.

In some embodiments, the invention relates generally to non-invasive methods, diagnostic tests, especially blood (including serum or plasma) tests that measure biomarkers (e.g. methylation profile and/or protein profile), and computer-implemented machine learning methods, apparatuses, systems, and computer-readable media for assessing a likelihood that a patient has a disease, such as cancer, relative to a patient population or a cohort population to determine whether that patient should be followed up with additional, more invasive testing.

In some embodiments, the technology provides for non-invasive methods, diagnostic tests, and computer-implemented machine learning methods, apparatuses, systems, and computer-readable media for assessing a likelihood that a patient has a disease, such as cancer, relative to a population or a cohort population by generating, e.g., stratified risk categories to more accurately predict the presence of cancer in an otherwise asymptomatic or vaguely symptomatic patient.

In some embodiments, the test provides a risk categorization of a population or cohort population of individuals is used to determine a quantified risk level for the presence of a cancer in an asymptomatic human subject. In some aspects, data used to determine the risk level may include, but is not limited to, a blood test that measures multiple biomarkers in the blood.

### Methods of Use

### Methods of Diagnosis of a Subject

Disclosed herein, in certain embodiments, are methods of diagnosing liver cancer and selecting subjects suspected of having liver cancer for treatment. In some instances, the methods comprise utilizing one or more biomarkers described herein. In some instances, a biomarker comprises a cytosine methylation site. In some instances, cytosine methylation comprises 5-methylcytosine (5-mCyt) and 5-hydroxymethylcytosine. In some cases, a cytosine methylation site occurs in a CpG dinucleotide motif. In other cases, a cytosine methylation site occurs in a CHG or CHH motif, in which H is adenine, cytosine or thymine. In some instances, one or more CpG dinucleotide motif or CpG site forms a CpG island, a short DNA sequence rich in CpG dinucleotide. In some instances, CpG islands are typically, but not always, between about 0.2 to about 1 kb in length. In some instances, a biomarker comprises a CpG island.

In some embodiments, disclosed herein is a method of selecting a subject suspected of having liver cancer for treatment, in which the method comprises (a) processing an extracted genomic DNA with a deaminating agent to generate a genomic DNA sample comprising deaminated nucleotides, wherein the extracted genomic DNA is obtained from a biological sample from the subject suspected of having liver cancer; (b) generating a methylation profile comprising one or more biomarkers selected from the Tables 1, 2, and 6; (c) comparing the methylation profile of the one or more biomarkers with a control; (d) identifying the subject as having liver cancer if the methylation profile correlates to the control; and (e) administering an effective amount of a therapeutic agent to the subject if the subject is identified as having liver cancer.

In some embodiments, the method is related to screening for HCC in a sample obtained from a subject, the method comprising assaying a methylation state of a marker in a sample obtained from a subject; and identifying the subject as having HCC when the methylation state of the marker is different than a methylation state of the marker assayed in a subject that does not have HCC (e.g., a subject that does not have HCC) (e.g., a subject that does not have HCC but does have liver cirrhosis), wherein the marker comprises one or more bases in a differentially methylated region (DMR) selected from UBE4B, TNFAIP8L2 (SCNM1), RASSF5, RPS6KA1, IFITM1, PPFIA1, SYT9, EVL, C16orf54, VMP1, OGFOD3, PSD4, KIAA0930, BDH1, F12, H4C6, LOC100287329, FOXP4 (AS1), PTPRN2, YZ2 (MYO1G), LAT2, MAP3K8, HEXDC, CTTN, LTA, LOC101928253, URI1, LINC01298, HIST1H4F, MIR153-2, PFN3, LOC101929153, MIR1302-7, LOC100506585, DIRAS1, and MIR21 as provided in Tables 1, 2, and 6. In some embodiments, the marker comprises one or more bases in a DMR selected from UBE4B, TNFAIP8L2 (SCNM1), RASSF5, RPS6KA1, IFITM1, EVL, C16orf54, PSD4, KIAA0930, BDH1, FOXP4 (AS1), YZ2 (MYO1G), LAT2, MAP3K8, HEXDC, CTTN, LTA, LOC101928253, URI1, LINC01298, HIST1H4F, MIR153-2, PFN3, LOC101929153, MIR1302-7, LOC100506585, DIRAS1, and MIR21 as provided in Tables 1, 2, and 6. In some embodiments, the marker comprises one or more bases in a DMR selected from UBE4B, TNFAIP8L2 (SCNM1), RASSF5, RPS6KA1, IFITM1, PPFIA1, SYT9, EVL, C16orf54, VMP1, OGFOD3, PSD4, KIAA0930, BDH1, F12, H4C6, LOC100287329, FOXP4 (AS1), PTPRN2, YZ2 (MYO1G), and LAT2 as provided in Tables 1, 2 and 6.

In some embodiments, the markers and/or panels of markers were identified (e.g., a chromosomal region having an annotation provided in Tables 2, 3, 4, 5, and 6) capable of detecting HCC (see, Examples I, and II) (e.g., UBE4B, TNFAIP8L2 (SCNM1), RASSF5, RPS6KA1, IFITM1, PPFIA1, SYT9, EVL, C16orf54, VMP1, OGFOD3, PSD4, KIAA0930, BDH1, F12, H4C6, LOC100287329, FOXP4 (AS1), PTPRN2, YZ2 (MYO1G), and LAT2).

Although the disclosure herein refers to certain illustrated embodiments, it is to be understood that these embodiments are presented by way of example and not by way of limitation.

In some embodiments, a methylation profile comprises a plurality of CpG methylation data for one or more biomarkers described herein. In some instances, a plurality of CpG methylation data is generated by first obtaining a genomic DNA (e.g., nuclear DNA or circulating DNA) from a biological sample, and then treating the genomic DNA by a deaminating agent to generate an extracted genomic DNA. In some instances, the extracted genomic DNA (e.g., extracted nuclear DNA or extracted circulating DNA) is optionally treated with one or more restriction enzymes to generate a set of DNA fragments prior to submitting for sequencing analysis to generate CpG methylation data. In some cases, the sequencing analysis comprises hybridizing each of the one or more biomarkers described herein with a probe, and performing a DNA sequencing reaction to quantify the methylation of each of the one or more biomarkers. In some instances, the CpG methylation data is then input into a machine learning/classification program to generate a methylation profile.

In some instances, a set of biological samples are generated and subsequently input into the machine learning/classification program. In some instances, the set of biological samples comprises 2, 3, 4, 5, 6, 7, 8, 9, 10, 20, 30, or more biological samples. In some instances, the set of biological samples comprises 2, 3, 4, 5, 6, 7, 8, 9, 10, 20, 30, or more normal biological samples. In some instances, the set of biological samples comprises 2, 3, 4, 5, 6, 7, 8, 9, 10, 20, 30, or more cancerous biological samples. In some cases, the set of biological samples comprise a biological sample of interest, a first primary cancer sample, a second primary cancer sample, a first normal sample, a second normal sample, and a third normal sample; wherein the first, and second primary cancer samples are different; and wherein the first, second, and third normal samples are different. In some cases, three pairs of difference datasets are generated in which the three pairs of dataset comprise: a first difference dataset between the methylation profile of the biological sample of interest and the first normal sample, in which the biological sample of interest and the first normal sample are from the same biological sample source; a second difference dataset between a methylation profile of a second normal sample and a methylation profile of a third normal sample, in which the second and third normal samples are different; and a third difference dataset between a methylation profile of a first primary cancer sample and a methylation profile of a second primary cancer sample, in which the first and second primary cancer samples are different. In some instances, the difference datasets are further input into the machine learning/classification program. In some cases, a pair-wise methylation difference dataset from the first, second, and third datasets is generated and then analyzed in the presence of a control dataset or a training dataset by the machine learning/classification method to generate the cancer CpG methylation profile. In some instances, the first primary cancer sample is a liver cancer sample. In some cases, the second primary cancer sample is a non-liver cancer sample. In some cases, the machine learning method comprises identifying a plurality of markers and a plurality of weights based on a top score (e.g., a t-test value, a R test value), and classifying the samples based on the plurality of markers and the plurality of weights. In some cases, the machine learning method utilizes an algorithm selected from one or more of the following: a principal component analysis, a logistic regression analysis, a nearest neighbor analysis, a support vector machine, and a neural network model.

In some embodiments, the CpG methylation profile comprises one or more biomarkers selected from the Table 2. In some embodiments, the CpG methylation profile comprises two or more biomarkers selected from the Table 6.

In some instances, the subject is diagnosed in having liver cancer. In some instances, liver cancer further comprises a relapsed or refractory liver cancer. In other instances, liver cancer comprises a metastatic liver cancer. In some cases, the subject is diagnosed in having a relapsed or refractory liver cancer. In additional cases, the subject is diagnosed in having a metastatic liver cancer.

In some embodiments, a liver cancer is any type of liver cancer. In some instances, a liver cancer comprises hepatocellular carcinoma (HCC), fibrolamellar HCC, cholangiocarcinoma, angiosarcoma, or hepatoblastoma.

In some embodiments, the subject diagnosed of having liver cancer is further treated with a therapeutic agent. Exemplary therapeutic agents include, but are not limited to, sorafenib tosylate, doxorubicin, fluorouracil, cisplatin, or a combination thereof.

In certain embodiments, provided herein is a method of generating a methylation profile of a biomarker in a subject in need thereof, comprising: (a) processing an extracted genomic DNA with a deaminating agent to generate a genomic DNA sample comprising deaminated nucleotides, wherein the extracted genomic DNA is obtained from a biological sample from the subject; (b) detecting a hybridization between the extracted genomic DNA and a probe, wherein the probe hybridizes to a biomarker selected from Table 1; and (c) generating a methylation profile based on the detected hybridization between the extracted genomic DNA and the probe.

In some instances, as described elsewhere herein, a pair-wise methylation difference dataset is generated prior to generating a methylation profile. In some cases, the pair-wise methylation difference dataset comprises (i) a first difference between the methylation profile of the treated genomic DNA with a methylation profile of a first normal sample; (ii) a second difference between a methylation profile of a second normal sample and a methylation profile of a third normal sample; and (iii) a third difference between a methylation profile of a first primary cancer sample and a methylation profile of a second primary cancer sample.

In some cases, the pair-wise methylation difference dataset is analyzed with a control by a machine learning method to generate a methylation profile. In some cases, the machine learning method utilizes an algorithm selected from one or more of the following: a principal component analysis, a logistic regression analysis, a nearest neighbor analysis, a support vector machine, and a neural network model.

In some embodiments, a probe comprises a DNA probe thereof. In some instances, a probe comprises natural nucleic acid molecules and non-natural nucleic acid molecules. In some cases, a probe comprises a labeled probe, such as for example, fluorescently labeled probe or radioactively labeled probe. In some instances, a probe correlates to a CpG site. In some instances, a probe is utilized in a next generation sequencing reaction to generate a CpG methylation data. In further instances, a probe is used in a solution-based next generation sequencing reaction to generate a CpG methylation data.

In some cases, a probe comprises 120 bp or more bases.

In one aspect, the invention features a composition for nucleic acid hybridization.

In some embodiments, the composition further comprises a capture probe oligonucleotide, the capture probe oligonucleotide comprising a region that is complementary to a portion of a strand of genomic DNA. The capture probe is not limited to any particular configuration. In certain preferred embodiments, the capture probe oligonucleotide comprises a region that is complementary to a portion of bisulfite or enzymatic converted DNA or the complement thereof.

The kit further provides methods of characterizing samples. In some embodiments, the method comprises a) treating DNA from a sample with a bisulfite reagent or enzymes to produce bisulfite or enzymatic-converted DNA, and b) amplifying a region of the bisulfite-converted DNA.

The present invention pertains to methods of purifying a target molecule contained within a test sample. Typically, the target molecule in a test sample will be a nucleic acid molecule, in particular, single-stranded DNA or bisulfite or enzymatically converted DNA.

In some cases, the method further comprises performing a DNA sequencing reaction such as those described elsewhere herein to quantify the methylation of each of the one or more biomarkers prior to generating a methylation profile.

In some embodiments, a CpG methylation site is located at the promoter region (e.g., induces a promoter methylation). In some instances, promoter methylation leads to a downregulation of its corresponding gene expression. In some instances, one or more CpG methylation sites described supra and in subsequent paragraphs are located at promoter regions, leading to promoter methylation, and subsequent downregulation of the corresponding gene expression. In some instances, the CpG methylation site is as illustrated in Tables 6, or list of genes from the Table 1. In some cases, an increase in gene expression leads to a decrease in tumor volume.

In some embodiments, the method comprising generating a methylation profile comprising one or more genes selected from: UBE4B, TNFAIP8L2 (SCNM1), RASSF5, RPS6KA1, IFITM1, PPFIA1, SYT9, EVL, C16orf54, VMP1, OGFOD3, PSD4, KIAA0930, BDH1, F12, H4C6, LOC100287329, FOXP4 (AS1), PTPRN2, YZ2 (MYO1G), LAT2, MAP3K8, HEXDC, CTTN, LTA, LOC101928253, URI1, LINC01298, HIST1H4F, MIR153-2, PFN3, LOC101929153, MIR1302-7, LOC100506585, DIRAS1, and MIR21.

In some embodiments, the method comprising generating a methylation profile comprising one or more genes selected from: UBE4B, TNFAIP8L2 (SCNM1), RASSF5, RPS6KA1, IFITM1, EVL, C16orf54, PSD4, KIAA0930, BDH1, FOXP4 (AS1), YZ2 (MYO1G), LAT2, MAP3K8, HEXDC, CTTN, LTA, LOC101928253, URI1, LINC01298, HIST1H4F, MIR153-2, PFN3, LOC101929153, MIR1302-7, LOC100506585, DIRAS1, and MIR21.

In some embodiments, described herein is a method of selecting a subject suspected of having liver cancer for treatment, the method comprising generating a methylation profile comprising one or more genes selected from: UBE4B, TNFAIP8L2 (SCNM1), RASSF5, RPS6KA1, IFITM1, PPFIA1, SYT9, EVL, C16orf54, VMP1, OGFOD3, PSD4, KIAA0930, BDH1, F12, H4C6, LOC100287329, FOXP4 (AS1), PTPRN2, YZ2 (MYO1G), and LAT2.

In some instances, the methylation profile comprises one or more genes selected from the Table 1, 2, or 6. In some instances, the methylation profile comprises one or more genes selected from the Table 1. In some instances, the methylation profile comprises one or more genes selected from the Table 2. In some instances, the methylation profile comprises one or more genes selected from the Table 6.

### Determining the Protein Profile

In some embodiments, disclosed herein include a method of determining the protein profile for risk scoring, the method comprises: a) obtaining hepatocellular carcinoma protein marker profile for a specimen obtained from the subject. b) comparing the protein marker profile to a control group.

In some embodiments, to evaluate whether a subject has HCC, the presence of one or more HCC protein markers in a sample is assessed to produce a profile, and that profile is compared to a control profile to evaluate HCC. The HCC protein marker profile may be employed to distinguish subjects having HCC from subjects having cirrhosis.

While a wide range of proteins may be employed as HCC protein markers, the HCC protein markers employed in many embodiments of the instant methods include proteins selected from the group consisting of: AFP, Lens culinaris agglutinin-reactive AFP (AFP-L3), des-gamma carboxy prothrombin (DCP), osteopontin, midkine (MDK), dikkopf-1 (DKK1), glypican-3 (GPC-3), alpha-1 fucosidase (AFU), and golgi protein-73 (GP-73).

In certain embodiments, the instant methods include: obtaining an HCC protein marker profile or multiple protein markers profile that include quantitative data for at least one protein marker selected from the group consisting of AFP, AFP-L3, and DCP, and comparing the profile with a control profile.

In certain embodiments, the method may further include evaluating AFP, DCP and AFP-L3 levels.

In certain embodiments, the protein panel thereof may be used in diagnostic methods and in in vitro assays to detect the presence of HCC.

The method includes a step or multiple steps of performing ELISA or other automated immunoassay analyzers (e.g., microfluidic electrophoretic device) on a blood-based sample obtained from the patient.

In some embodiments, the candidate protein markers demonstrated test set performance of clinical relevance in screening of patients at high risk for developing HCC. The protein panel performance seemed effective to detect underlying liver disease within the range of etiologies studied, which spanned the most common causes of liver disease in the United States population. The high performance extended to detection of small lesions of less than 2 cm or TNM stage T1. This is important as for any HCC screening program to impact patient survival, the cancer be identified as early as possible, when effective therapies can be offered to newly diagnosed patients.

In some embodiments, the multi-analyte test comprising multiple biomarkers encompassing DNA methylation and the three-protein markers. The multi-analyte panel performance seemed superior over the other methods. The high performance extended to detection of small lesions of less than 2 cm or TNM stage T1.

### Combination of Methylation and Three-Protein Scores

In some instances, a methylation score is utilized to determine the diagnosis of a subject. In some instances, diagnosis refers to the prediction of the likelihood of cancer-attributable death or progression, including recurrence, metastatic spread, and drug resistance, of liver cancer. The term “prediction” is used herein to refer to the likelihood that a subject will respond either favorably or unfavorably to a drug or set of drugs, and also the extent of those responses, or that a subject will survive, following chemotherapy for a certain period of time without cancer recurrence and/or following surgery (e.g., removal of the spleen). In some instances, a methylation score is utilized to determine the prognosis of a subject having liver cancer.

As such, “making a diagnosis” or “diagnosing”, as used herein, is further inclusive of making determining a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the measure of the diagnostic biomarkers (e.g., DMR) disclosed herein. Further, in some embodiments of the presently disclosed subject matter, multiple determinations of the biomarkers over time can be made to facilitate diagnosis and/or prognosis. A temporal change in the biomarker can be used to predict a clinical outcome, monitor the progression of HCC, and/or monitor the efficacy of appropriate therapies directed against the cancer. In such an embodiment for example, one might expect to see a change in the methylation state of one or more biomarkers (e.g., DMR) disclosed herein (and potentially one or more additional biomarker(s), if monitored) in a biological sample over time during the course of an effective therapy.

Thus, the present method and risk score are based, at least in part, on 1) the identification and clustering of a set of proteins and/or resulting methylation levels of specific genes that can serve as markers for the presence of a cancer, 2) normalization and aggregation of the markers measured to generate a biomarker composite score; and, 3) medical data for a patient and other publicly available sources of data for risk factors for having cancer; and (4) determination of threshold values used to divide patients into groups with varying degrees of risk for the presence of cancer in which the likelihood of an asymptomatic human subject having a quantified increased risk for the presence of the cancer is determined. A machine learning system may be utilized to determine the best cohort grouping as well as determine how biomarker composite data, medical data and other data are to be combined in order to generate a risk categorization in an optimal or near-optimal manner, e.g., correctly predicting which individuals have cancer with a low false positive rate. The machine learning system yields a numerical risk score for each patient tested, which can be used by physicians to make treatment decisions concerning the therapy of cancer patients or, importantly, to further inform screening procedures to better predict and diagnose early-stage cancer in asymptomatic patients.

Also, as described in more detail herein, the machine learning system is adapted to receive additional data as the system is used in a real-world clinical setting and to recalculate and improve the risk categories and algorithm so that the system becomes “smarter” the more that it is used.

In some embodiments, a statistical analysis associates diagnostic or prognostic indicators with a predisposition to an adverse outcome. For example, in some embodiments, a methylation state different from that in a normal control sample obtained from a patient who does not have a disorder can signal that a subject is more likely to suffer from a disorder than subjects with a level that is more similar to the methylation state in the control sample, as determined by a level of statistical significance.

In some instances, the combination of methylation and the three-protein score can be used for final prediction.

In some instances, the methylation markers and three-protein markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

### Control

In some embodiments, a control is a methylation value, methylation level, or methylation profile of a sample. In some instances, the control comprises a set of methylation profiles, wherein each said methylation profile is generated from a biological sample obtained from a known cancer type. In some cases, the known cancer type is liver cancer. In some cases, the known cancer type is a relapsed or refractory liver cancer. In other cases, the known cancer type is a metastatic liver cancer. In some cases, the known cancer type is hepatocellular carcinoma (HCC), fibrolamellar HCC, cholangiocarcinoma, angiosarcoma, or hepatoblastoma.

In some embodiments, various other control groups include those with non-cancerous liver disorders, benign liver diseases, normal controls (controls with and without cirrhosis) and other cancers.

In some embodiments of the method, the control methylation state is any detectable methylation state of the biomarker. In other embodiments of the method where a control sample is tested concurrently with the biological sample, the predetermined methylation state is the methylation state in the control sample.

In some embodiments, a control can be DNA or oligonucleotides for use in ensuring that components of reactions are functioning properly.

In some embodiments, a control also relates to use of endogenous methylated DNAs as internal controls for marker gene methylation assays (e.g., markers in Table 5).

In some embodiments, a control relates to methylated control DNA that can be processed and detected alongside methylated marker DNA indicative of disease. A control nucleic acid comprising a sequence from a DMR selected from the Table 6, and having a methylation state associated with a subject who does not have a cancer.

### Probes for Capture

In some embodiments, the constituents of the kit are the same as for the method disclosed above. The DNA hybridization probes are preferably specific for target sequences selected from the group of specific regions. The specific regions are preferably selected from the group of metabolic genes, regulatory genes and oncogenes. The capture DNA probes may be synthesized DNA probes. Alternatively, the DNA probes may be isolated and purified from a biological sample.

### Detection Methods

In some embodiments, a number of methods are utilized to measure, detect, determine, identify, and characterize the methylation status/level of a gene or a biomarker (e.g., CpG island-containing region/fragment) in identifying a subject as having liver cancer, determining the liver cancer subtype, the prognosis of a subject having liver cancer, and the progression or regression of liver cancer in subject in the presence of a therapeutic agent.

In some instances, the methylation profile is generated from a biological sample isolated from an individual. In some embodiments, the biological sample is a biopsy. In some instances, the biological sample is a tissue sample. In some instances, the biological sample is a tissue biopsy sample. In some instances, the biological sample is a blood sample. In other instances, the biological sample is a cell-free biological sample. In other instances, the biological sample is a cell-free DNA sample. In other instances, the biological sample is a circulating tumor DNA sample. In one embodiment, the biological sample is a cell free biological sample containing circulating tumor DNA.

In some embodiments, a biomarker (or an epigenetic marker) is obtained from a liquid sample. In some embodiments, the liquid sample comprises blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some embodiments, the biological fluid is blood, a blood derivative or a blood fraction, e.g., serum or plasma. In a specific embodiment, a sample comprises a blood sample. In another embodiment, a serum sample is used. In another embodiment, a sample comprises urine. In some embodiments, the liquid sample also encompasses a sample that has been manipulated in any way after their procurement, such as by centrifugation, filtration, precipitation, dialysis, chromatography, treatment with reagents, washed, or enriched for certain cell populations.

In some embodiments, a biomarker (or an epigenetic marker) is obtained from a tissue sample. In some instances, a tissue corresponds to any cell(s). Different types of tissue correspond to different types of cells (e.g., liver, lung, blood, connective tissue, and the like), but also healthy cells vs. tumor cells or to tumor cells at various stages of neoplasia, or to displaced malignant tumor cells. In some embodiments, a tissue sample further encompasses a clinical sample, and also includes cells in culture, cell supernatants, organs, and the like. Samples also comprise fresh-frozen and/or formalin-fixed, paraffin-embedded tissue blocks, such as blocks prepared from clinical or pathological biopsies, prepared for pathological analysis or study by immunohistochemistry.

In some embodiments, a biomarker (or an epigenetic marker) is methylated or unmethylated in a normal sample (e.g., normal or control tissue without disease, or normal or control body fluid, stool, blood, serum, amniotic fluid), most importantly in healthy stool, blood, serum, amniotic fluid or other body fluid. In other embodiments, a biomarker (or an epigenetic marker) is hypomethylated or hypermethylated in a sample from a patient having or at risk of a disease (e.g., one or more indications described herein); for example, at a decreased or increased (respectively) methylation frequency of at least about 50%, at least about 60%, at least about 70%, at least about 75%, at least about 80%, at least about 85%, at least about 90%, at least about 95%, or about 100% in comparison to a normal sample. In one embodiment, a sample is also hypomethylated or hypermethylated in comparison to a previously obtained sample analysis of the same patient having or at risk of a disease (e.g., one or more indications described herein), particularly to compare progression of a disease.

In some embodiments, a methylome comprises a set of epigenetic markers or biomarkers, such as a biomarker described above. In some instances, a methylome that corresponds to the methylome of a tumor of an organism (e.g., a human) is classified as a tumor methylome. In some cases, a tumor methylome is determined using tumor tissue or cell-free (or protein-free) tumor DNA in a biological sample. Other examples of methylomes of interest include the methylomes of organs that contribute DNA into a bodily fluid (e.g. methylomes of tissue such as brain, breast, lung, the prostate, and the kidneys, plasma, etc.).

In some embodiments, a plasma methylome is the methylome determined from the plasma or serum of an animal (e.g., a human). In some instances, the plasma methylome is an example of a cell-free or protein-free methylome since plasma and serum include cell-free DNA. The plasma methylome is also an example of a mixed methylome since it is a mixture of tumor and other methylomes of interest. In some instances, the urine methylome is determined from the urine sample of a subject. In some cases, a cellular methylome corresponds to the methylome determined from cells (e.g., blood cells) of the patient. The methylome of the blood cells is called the blood cell methylome (or blood methylome).

In some embodiments, DNA (e.g., genomic DNA such as extracted genomic DNA or treated genomic DNA) is isolated by any means standard in the art, including the use of commercially available kits. Briefly, wherein the DNA of interest is encapsulated in by a cellular membrane the biological sample is disrupted and lysed by enzymatic, chemical or mechanical means. In some cases, the DNA solution is then cleared of proteins and other contaminants e.g. by digestion with proteinase K. The DNA is then recovered from the solution. In such cases, this is carried out by means of a variety of methods including salting out, organic extraction or binding of the DNA to a solid phase support. In some instances, the choice of method is affected by several factors including time, expense and required quantity of DNA.

Wherein the sample DNA is not enclosed in a membrane (e.g. circulating DNA from a cell free sample such as blood or urine) methods standard in the art for the isolation and/or purification of DNA are optionally employed (See, for example, Bettegowda et al. Detection of Circulating Tumor DNA in Early- and Late-Stage Human Malignancies. Sci. Transl. Med, 6(224): ra24. 2014). Such methods include the use of a protein degenerating reagent e.g. chaotropic salt e.g. guanidine hydrochloride or urea; or a detergent e.g. sodium dodecyl sulphate (SDS), cyanogen bromide. Alternative methods include but are not limited to ethanol precipitation or propanol precipitation, vacuum concentration amongst others by means of a centrifuge. In some cases, the person skilled in the art also make use of devices such as filter devices e.g. ultrafiltration, silica surfaces or membranes, magnetic particles, polystyrol particles, polystyrol surfaces, positively charged surfaces, and positively charged membranes, charged membranes, charged surfaces, charged switch membranes, charged switched surfaces.

In some instances, once the nucleic acids have been extracted, methylation analysis is carried out by any means known in the art. A variety of methylation analysis procedures are known in the art and may be used to practice the methods disclosed herein. These assays allow for determination of the methylation state of one or a plurality of CpG sites within a tissue sample. In addition, these methods may be used for absolute or relative quantification of methylated nucleic acids. Such methylation assays involve, among other techniques, two major steps. The first step is a methylation specific reaction or separation, such as (i) bisulfite treatment, (ii) methylation specific binding, or (iii) methylation specific restriction enzymes. The second major step involves (i) amplification and detection, or (ii) direct detection, by a variety of methods such as (a) PCR (sequence-specific amplification) such as Tagman®, (b) DNA sequencing of untreated and bisulfite-treated DNA, (c) sequencing by ligation of dye-modified probes (including cyclic ligation and cleavage), (d) pyrosequencing, (e) single-molecule sequencing, (f) mass spectroscopy, or (g) Southern blot analysis.

Additionally, restriction enzyme digestion of PCR products amplified from bisulfite-converted DNA may be used, e.g., the method described by Sadri and Hornsby (1996, Nucl. Acids Res. 24:5058-5059), or COBRA (Combined Bisulfite Restriction Analysis) (Xiong and Laird, 1997, Nucleic Acids Res. 25:2532-2534). COBRA analysis is a quantitative methylation assay useful for determining DNA methylation levels at specific gene loci in small amounts of genomic DNA. Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Frommer et al, 1992, Proc. Nat. Acad. Sci. USA, 89, 1827-1831). PCR amplification of the bisulfite converted DNA is then performed using primers specific for the CpG sites of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from micro-dissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides. Additionally, bisulfite conversion reagents may include: DNA denaturation buffer; sulfo nation buffer; DNA recovery reagents or kits (e.g., precipitation, ultrafiltration, affinity column); desulfonation buffer; and DNA recovery components.

In an embodiment, the methylation profile of selected CpG sites is determined using methylation-Specific PCR (MSP). MSP allows for assessing the methylation status of virtually any group of CpG sites within a CpG island, independent of the use of methylation-sensitive restriction enzymes (Herman et al, 1996, Proc. Nat. Acad. Sci. USA, 93, 9821-9826; U.S. Pat. Nos. 5,786,146, 6,017,704, 6,200,756, 6,265,171 (Herman and Baylin); U.S. Pat. Pub. No. 2010/0144836 (Van Engeland et al)). Briefly, DNA is modified by a deaminating agent such as sodium bisulfite to convert unmethylated, but not methylated cytosines to uracil, and subsequently amplified with primers specific for methylated versus unmethylated DNA. In some instances, typical reagents (e.g., as might be found in a typical MSP-based kit) for MSP analysis include, but are not limited to: methylated and unmethylated PCR primers for specific gene (or methylation-altered DNA sequence or CpG island), optimized PCR buffers and deoxynucleotides, and specific probes. One may use quantitative multiplexed methylation specific PCR (QM-PCR), as described by Fackler et al. Fackler et al, 2004, Cancer Res. 64(13) 4442-4452; or Fackler et al, 2006, Clin. Cancer Res. 12(11 Pt 1) 3306-3310.

In an embodiment, the methylation profile of selected CpG sites is determined using MethyLight and/or Heavy Methyl Methods. The MethyLight and Heavy Methyl assays are a high-throughput quantitative methylation assay that utilizes fluorescence-based real-time PCR (Taq Man®) technology that requires no further manipulations after the PCR step (Eads, C. A. et al, 2000, Nucleic Acid Res. 28, e 32; Cottrell et al, 2007, J. Urology 177, 1753, U.S. Pat. No. 6,331,393 (Laird et al)). Briefly, the MethyLight process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. In some cases, sequence discrimination occurs either at the level of the amplification process or at the level of the fluorescence detection process, or both. In some cases, the MethyLight assay is used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites. Typical reagents (e.g., as might be found in a typical MethyLight-based kit) for MethyLight analysis may include, but are not limited to: PCR primers for specific gene (or methylation-altered DNA sequence or CpG island); TaqMan® probes; optimized PCR buffers and deoxynucleotides; and Taq polymerase.

Quantitative MethyLight uses bisulfite to convert genomic DNA and the methylated sites are amplified using PCR with methylation independent primers. Detection probes specific for the methylated and unmethylated sites with two different fluorophores provides simultaneous quantitative measurement of the methylation. The Heavy Methyl technique begins with bisulfate conversion of DNA. Next specific blockers prevent the amplification of unmethylated DNA. Methylated genomic DNA does not bind the blockers and their sequences will be amplified. The amplified sequences are detected with a methylation specific probe. (Cottrell et al, 2004, Nuc. Acids Res. 32:e10, the contents of which is hereby incorporated by reference in its entirety).

The Ms-SNuPE technique is a quantitative method for assessing methylation differences at specific CpG sites based on bisulfite treatment of DNA, followed by single-nucleotide primer extension (Gonzalgo and Jones, 1997, Nucleic Acids Res. 25, 2529-2531). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. In some cases, small amounts of DNA are analyzed (e.g., micro-dissected pathology sections), and the method avoids utilization of restriction enzymes for determining the methylation status at CpG sites. Typical reagents (e.g., as is found in a typical Ms-SNuPE-based kit) for Ms-SNuPE analysis include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

In another embodiment, the methylation status of selected CpG sites is determined using differential Binding-based Methylation Detection Methods. For identification of differentially methylated regions, one approach is to capture methylated DNA. This approach uses a protein, in which the methyl binding domain of MBD2 is fused to the Fc fragment of an antibody (MBD-FC) (Gebhard et al, 2006, Cancer Res. 66:6118-6128; and PCT Pub. No. WO 2006/056480 A2 (Relhi)). This fusion protein has several advantages over conventional methylation specific antibodies. The MBD FC has a higher affinity to methylated DNA and it binds double stranded DNA. Most importantly the two proteins differ in the way they bind DNA. Methylation specific antibodies bind DNA stochastically, which means that only a binary answer can be obtained. The methyl binding domain of MBD-FC, on the other hand, binds DNA molecules regardless of their methylation status. The strength of this protein-DNA interaction is defined by the level of DNA methylation. After binding genomic DNA, eluate solutions of increasing salt concentrations can be used to fractionate non-methylated and methylated DNA allowing for a more controlled separation (Gebhard et al, 2006, Nucleic Acids Res. 34: e82). Consequently this method, called Methyl-CpG immunoprecipitation (MCIP), not only enriches, but also fractionates genomic DNA according to methylation level, which is particularly helpful when the unmethylated DNA fraction should be investigated as well.

In an alternative embodiment, a 5-methyl cytidine antibody to bind and precipitate methylated DNA. Antibodies are available from Abeam (Cambridge, MA), Diagenode (Sparta, NJ) or Eurogentec (c/o AnaSpec, Fremont, CA). Once the methylated fragments have been separated they may be sequenced using microarray based techniques such as methylated CpG-island recovery assay (MIRA) or methylated DNA immunoprecipitation (MeDIP) (Pelizzola et al, 2008, Genome Res. 18, 1652-1659; O'Geen et al, 2006, BioTechniques 41(5), 577-580, Weber et al, 2005, Nat. Genet. 37, 853-862; Horak and Snyder, 2002, Methods Enzymol, 350, 469-83; Lieb, 2003, Methods Mol Biol, 224, 99-109). Another technique is methyl-CpG binding domain column/segregation of partly melted molecules (MBD/SPM, Shiraishi et al, 1999, Proc. Natl. Acad. Sci. USA 96(6):2913-2918).

In some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. See, e.g., U.S. Pat. No. 7,186,512. Alternatively, the method can encompass amplifying intact DNA after restriction enzyme digestion, thereby only amplifying DNA that was not cleaved by the restriction enzyme in the area amplified. See, e.g., U.S. Pat. Nos. 7,910,296; 7,901,880; and 7,459,274. In some embodiments, amplification can be performed using primers that are gene specific.

For example, there are methyl-sensitive enzymes that preferentially or substantially cleave or digest at their DNA recognition sequence if it is non-methylated. Thus, an unmethylated DNA sample is cut into smaller fragments than a methylated DNA sample. Similarly, a hypermethylated DNA sample is not cleaved. In contrast, there are methyl-sensitive enzymes that cleave at their DNA recognition sequence only if it is methylated. Methyl-sensitive enzymes that digest unmethylated DNA suitable for use in methods of the technology include, but are not limited to, Hpall, Hhal, Maell, BstUI and Acil. In some instances, an enzyme that is used is Hpall that cuts only the unmethylated sequence CCGG. In other instances, another enzyme that is used is Hhal that cuts only the unmethylated sequence GCGC. Both enzymes are available from New England BioLabs®, Inc. Combinations of two or more methyl-sensitive enzymes that digest only unmethylated DNA are also used. Suitable enzymes that digest only methylated DNA include, but are not limited to, Dpnl, which only cuts at fully methylated 5′-GATC sequences, and McrBC, an endonuclease, which cuts DNA containing modified cytosines (5-methylcytosine or 5-hydroxymethylcytosine or N4-methylcytosine) and cuts at recognition site 5′ . . . PumC(N4o-3ooo) PumC . . . 3′ (New England BioLabs, Inc., Beverly, MA). Cleavage methods and procedures for selected restriction enzymes for cutting DNA at specific sites are well known to the skilled artisan. For example, many suppliers of restriction enzymes provide information on conditions and types of DNA sequences cut by specific restriction enzymes, including New England BioLabs, Pro-Mega Biochems, Boehringer-Mannheim, and the like. Sambrook et al. (See Sambrook et al. Molecular Biology: A Laboratory Approach, Cold Spring Harbor, N.Y. 1989) provide a general description of methods for using restriction enzymes and other enzymes.

In some instances, a methylation-dependent restriction enzyme is a restriction enzyme that cleaves or digests DNA at or in proximity to a methylated recognition sequence, but does not cleave DNA at or near the same sequence when the recognition sequence is not methylated. Methylation-dependent restriction enzymes include those that cut at a methylated recognition sequence (e.g., Dpnl) and enzymes that cut at a sequence near but not at the recognition sequence (e.g., McrBC). For example, McrBC's recognition sequence is 5′ RmC (N40-3000) RmC 3′ where “R” is a purine and “mC” is a methylated cytosine and “N40-3000” indicates the distance between the two RmC half sites for which a restriction event has been observed. McrBC generally cuts close to one half-site or the other, but cleavage positions are typically distributed over several base pairs, approximately 30 base pairs from the methylated base. McrBC sometimes cuts 3′ of both half sites, sometimes 5′ of both half sites, and sometimes between the two sites. Exemplary methylation-dependent restriction enzymes include, e.g., McrBC, McrA, MrrA, Bisl, Glal and Dpnl. One of skill in the art will appreciate that any methylation-dependent restriction enzyme, including homologs and orthologs of the restriction enzymes described herein, is also suitable for use with one or more methods described herein.

In some cases, a methylation-sensitive restriction enzyme is a restriction enzyme that cleaves DNA at or in proximity to an unmethylated recognition sequence but does not cleave at or in proximity to the same sequence when the recognition sequence is methylated. Exemplary methylation-sensitive restriction enzymes are described in, e.g., McClelland et al, 22(17) NUCLEIC ACIDS RES. 3640-59 (1994). Suitable methylation-sensitive restriction enzymes that do not cleave DNA at or near their recognition sequence when a cytosine within the recognition sequence is methylated at position C5 include, e.g., Aat II, Aci I, Acd I, Age I, Alu I, Asc I, Ase I, AsiS I, Bbe I, BsaA I, BsaH I, BsiE I, BsiW I, BsrF I, BssH II, BssK I, BstB I, BstN I, BstU I, Cla I, Eae I, Eag I, Fau I, Fse I, Hha I, HinPl I, HinC II, Hpa II, Hpy99 I, HpyCH4 IV, Kas I, Mbo I, Mlu I, MapAl I, Msp I, Nae I, Nar I, Not I, Pml I, Pst I, Pvu I, Rsr II, Sac II, Sap I, Sau3A I, Sfl I, Sfo I, SgrA I, Sma I, SnaB I, Tsc I, Xma I, and Zra I. Suitable methylation-sensitive restriction enzymes that do not cleave DNA at or near their recognition sequence when an adenosine within the recognition sequence is methylated at position N6 include, e.g., Mbo I. One of skill in the art will appreciate that any methylation-sensitive restriction enzyme, including homologs and orthologs of the restriction enzymes described herein, is also suitable for use with one or more of the methods described herein. One of skill in the art will further appreciate that a methylation-sensitive restriction enzyme that fails to cut in the presence of methylation of a cytosine at or near its recognition sequence may be insensitive to the presence of methylation of an adenosine at or near its recognition sequence. Likewise, a methylation-sensitive restriction enzyme that fails to cut in the presence of methylation of an adenosine at or near its recognition sequence may be insensitive to the presence of methylation of a cytosine at or near its recognition sequence. For example, Sau3A1 is sensitive (i.e., fails to cut) to the presence of a methylated cytosine at or near its recognition sequence, but is insensitive (i.e., cuts) to the presence of a methylated adenosine at or near its recognition sequence. One of skill in the art will also appreciate that some methylation-sensitive restriction enzymes are blocked by methylation of bases on one or both strands of DNA encompassing of their recognition sequence, while other methylation-sensitive restriction enzymes are blocked only by methylation on both strands, but can cut if a recognition site is hemi-methylated.

In alternative embodiments, adaptors are optionally added to the ends of the randomly fragmented DNA, the DNA is then digested with a methylation-dependent or methylation-sensitive restriction enzyme, and intact DNA is subsequently amplified using primers that hybridize to the adaptor sequences. In this case, a second step is performed to determine the presence, absence or quantity of a particular gene in an amplified pool of DNA. In some embodiments, the DNA is amplified using real-time, quantitative PCR.

In other embodiments, the methods comprise quantifying the average methylation density in a target sequence within a population of genomic DNA. In some embodiments, the method comprises contacting genomic DNA with a methylation-dependent restriction enzyme or methylation-sensitive restriction enzyme under conditions that allow for at least some copies of potential restriction enzyme cleavage sites in the locus to remain uncleaved; quantifying intact copies of the locus; and comparing the quantity of amplified product to a control value representing the quantity of methylation of control DNA, thereby quantifying the average methylation density in the locus compared to the methylation density of the control DNA.

In some instances, the quantity of methylation of a locus of DNA is determined by providing a sample of genomic DNA comprising the locus, cleaving the DNA with a restriction enzyme that is either methylation-sensitive or methylation-dependent, and then quantifying the amount of intact DNA or quantifying the amount of cut DNA at the DNA locus of interest. The amount of intact or cut DNA will depend on the initial amount of genomic DNA containing the locus, the amount of methylation in the locus, and the number (i.e., the fraction) of nucleotides in the locus that are methylated in the genomic DNA. The amount of methylation in a DNA locus can be determined by comparing the quantity of intact DNA or cut DNA to a control value representing the quantity of intact DNA or cut DNA in a similarly-treated DNA sample. The control value can represent a known or predicted number of methylated nucleotides. Alternatively, the control value can represent the quantity of intact or cut DNA from the same locus in another (e.g., normal, non-diseased) cell or a second locus.

By using at least one methylation-sensitive or methylation-dependent restriction enzyme under conditions that allow for at least some copies of potential restriction enzyme cleavage sites in the locus to remain uncleaved and subsequently quantifying the remaining intact copies and comparing the quantity to a control, average methylation density of a locus can be determined. If the methylation-sensitive restriction enzyme is contacted to copies of a DNA locus under conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample. Such assays are disclosed in, e.g., U.S. Pat. No. 7,910,296.

The methylated CpG island amplification (MCA) technique is a method that can be used to screen for altered methylation patterns in genomic DNA, and to isolate specific sequences associated with these changes (Toyota et al, 1999, Cancer Res. 59, 2307-2312, U.S. Pat. No. 7,700,324 (Issa et al)). Briefly, restriction enzymes with different sensitivities to cytosine methylation in their recognition sites are used to digest genomic DNAs from primary tumors, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming Genomic DNA; PCR buffers and nucleotides, restriction enzymes and appropriate buffers; gene-hybridization oligos or probes; control hybridization oligos or probes.

In some embodiments, the methods provided herein further comprise performing the non-disruptive methylation sequencing technique. In some embodiments, the non-disruptive methylation sequencing technique is an enzymatic methyl-seq (EM-seq) technique. In some embodiments, the non-disruptive methylation sequencing technique comprises: (a) enzymatically modifying methylated cytosines (such as 5-methylcytosine (5 mc) and 5-hydroxymethylcytosine (5 hmC)) to prevent deamination in further enzymatic steps; (b) enzymatically converting unmethylated cytosines to uracils; (c) performing PCR amplification (thereby converting uracils to thymines; and (d) sequencing using a next generation sequencing technique. Various techniques for performing a non-disruptive methylation sequencing technique have been described in the art. See, e.g., Vaisvila et al., Genome Res, 31, 2021, which is incorporated herein in its entirety. In some embodiments, enzymatically modifying methylated cytosines is performed using TET2 and/or T4-BGT. In some embodiments, the non-disruptive methylation sequencing technique comprises enzymatically converting unmethylated cytosines to uracil using APOBEC3A. In some embodiments, the non-disruptive methylation sequencing technique comprises subjecting a sample comprising genomic DNA, such as a cfDNA sample, to a next generation sequencing library preparation technique. In some embodiments, the next generation sequencing library preparation technique comprises shearing the genomic DNA, such as to obtain a DNA size of less than about 500 base pairs, such as less than about any of 450 base pairs, 400 base pairs, 350 base pairs, or 300 base pairs. In some embodiments, the next generation sequencing library preparation technique comprises a step of end prep of sheared DNA. In some embodiments, the next generation sequencing library preparation technique comprises a step of adaptor ligation. In some embodiments, the next generation sequencing library preparation technique comprises a step of cleaning up adaptor ligated DNA. In some embodiments, the cleaned and ligated DNA is subjected to oxidative enzymes, such as TET2 and/or T4-BGT, to modify methylated cytosines (5-methylcytosines and 5-hydroxymethylcytosines). In some embodiments, the next generation sequencing library preparation technique comprises a step of cleaning enzyme oxidized DNA. In some embodiments, the oxidized DNA is further subjected to enzymatic cytosine deamination (such as using APOBEC3A). In some embodiments, the next generation sequencing library preparation technique comprises a step of PCR amplification of the deaminated DNA. In some embodiments, the next generation sequencing library preparation technique comprises a step of sequencing and quantification. In some embodiments, the method comprises adding a control to the sample comprising genomic DNA, e.g., prior to performing any enzymatic conversion steps.

Additional methylation detection methods include those methods described in, e.g., U.S. Pat. Nos. 7,553,627; 6,331,393; U.S. patent Ser. No. 12/476,981; U.S. Patent Publication No. 2005/0069879; Rein, et al, 26(10) NUCLEIC ACIDS RES. 2255-64 (1998); and Olek et al, 17(3) NAT. GENET. 275-6 (1997).

In another embodiment, the methylation status of selected CpG sites is determined using Methylation-Sensitive High Resolution Melting (HRM). Recently, Wojdacz et al. reported methylation-sensitive high resolution melting as a technique to assess methylation. (Wojdacz and Dobrovic, 2007, Nuc. Acids Res. 35(6) e41; Wojdacz et al. 2008, Nat. Prot. 3(12) 1903-1908; Balic et al, 2009 J. Mol. Diagn. 11 102-108; and US Pat. Pub. No. 2009/0155791 (Wojdacz et al)). A variety of commercially available real time PCR machines have HRM systems including the Roche LightCycler480, Corbett Research RotorGene6000, and the Applied Biosystems 7500. HRM may also be combined with other amplification techniques such as pyrosequencing as described by Candiloro et al. (Candiloro et al, 2011, Epigenetics 6(4) 500-507).

In another embodiment, the methylation status of selected CpG locus is determined using a primer extension assay, including an optimized PCR amplification reaction that produces amplified targets for analysis using mass spectrometry. The assay can also be done in multiplex. Mass spectrometry is a particularly effective method for the detection of polynucleotides associated with the differentially methylated regulatory elements. The presence of the polynucleotide sequence is verified by comparing the mass of the detected signal with the expected mass of the polynucleotide of interest. The relative signal strength, e.g., mass peak on a spectra, for a particular polynucleotide sequence indicates the relative population of a specific allele, thus enabling calculation of the allele ratio directly from the data. This method is described in detail in PCT Pub. No. WO 2005/012578A1 (Beaulieu et al), which is hereby incorporated by reference in its entirety. For methylation analysis, the assay can be adopted to detect bisulfite introduced methylation dependent C to T sequence changes. These methods are particularly useful for performing multiplexed amplification reactions and multiplexed primer extension reactions (e.g., multiplexed homogeneous primer mass extension (hME) assays) in a single well to further increase the throughput and reduce the cost per reaction for primer extension reactions.

Other methods for DNA methylation analysis include restriction landmark genomic scanning (RLGS, Costello et al, 2002, Meth. Mol Biol, 200, 53-70), methylation-sensitive-representational difference analysis (MS-RDA, Ushijima and Yamashita, 2009, Methods Mol Biol 507, 1 17-130). Comprehensive high-throughput arrays for relative methylation (CHARM) techniques are described in WO 2009/021141 (Feinberg and Irizarry). The Roche® NimbleGen® microarrays including the Chromatin Immunoprecipitation-on-chip (ChlP-chip) or methylated DNA immunoprecipitation-on-chip (MeDIP-chip). These tools have been used for a variety of cancer applications including melanoma, liver cancer and lung cancer (Koga et al, 2009, Genome Res., 19, 1462-1470; Acevedo et al, 2008, Cancer Res., 68, 2641-2651; Rauch et al, 2008, Proc. Nat. Acad. Sci. USA, 105, 252-257). Others have reported bisulfate conversion, padlock probe hybridization, circularization, amplification and next generation or multiplexed sequencing for high throughput detection of methylation (Deng et al, 2009, Nat. Biotechnol 27, 353-360; Ball et al, 2009, Nat. Biotechnol 27, 361-368; U.S. Pat. No. 7,611,869 (Fan)). As an alternative to bisulfate oxidation, Bayeyt et al. have reported selective oxidants that oxidize 5-methylcytosine, without reacting with thymidine, which are followed by PCR or pyro sequencing (WO 2009/049916 (Bayeyt et al).

In some instances, quantitative amplification methods (e.g., quantitative PCR or quantitative linear amplification) are used to quantify the amount of intact DNA within a locus flanked by amplification primers following restriction digestion. Methods of quantitative amplification are disclosed in, e.g., U.S. Pat. Nos. 6,180,349; 6,033,854; and 5,972,602, as well as in, e.g., DeGraves, et al, 34(1) BIOTECHNIQUES 106-15 (2003); Deiman B, et al., 20(2) MOL. BIOTECHNOL. 163-79 (2002); and Gibson et al, 6 GENOME RESEARCH 995-1001 (1996).

Following reaction or separation of nucleic acid in a methylation specific manner, the nucleic acid in some cases are subjected to sequence-based analysis. For example, once it is determined that one particular genomic sequence from a sample is hypermethylated or hypomethylated compared to its counterpart, the amount of this genomic sequence can be determined. Subsequently, this amount can be compared to a standard control value and used to determine the present of liver cancer in the sample. In many instances, it is desirable to amplify a nucleic acid sequence using any of several nucleic acid amplification procedures which are well known in the art. Specifically, nucleic acid amplification is the chemical or enzymatic synthesis of nucleic acid copies which contain a sequence that is complementary to a nucleic acid sequence being amplified (template). The methods and kits may use any nucleic acid amplification or detection methods known to one skilled in the art, such as those described in U.S. Pat. No. 5,525,462 (Takarada et al); U.S. Pat. No. 6,114,117 (Hepp et al); U.S. Pat. No. 6,127,120 (Graham et al); U.S. Pat. No. 6,344,317 (Urnovitz); U.S. Pat. No. 6,448,001 (Oku); U.S. Pat. No. 6,528,632 (Catanzariti et al); and PCT Pub. No. WO 2005/111209 (Nakajima et al).

In some embodiments, the nucleic acids are amplified by PCR amplification using methodologies known to one skilled in the art. One skilled in the art will recognize, however, that amplification can be accomplished by any known method, such as ligase chain reaction (LCR), Q-replicas amplification, rolling circle amplification, transcription amplification, self-sustained sequence replication, nucleic acid sequence-based amplification (NASBA), each of which provides sufficient amplification. Branched-DNA technology is also optionally used to qualitatively demonstrate the presence of a sequence of the technology, which represents a particular methylation pattern, or to quantitatively determine the amount of this particular genomic sequence in a sample. Nolte reviews branched-DNA signal amplification for direct quantitation of nucleic acid sequences in clinical samples (Nolte, 1998, Adv. Clin. Chem. 33:201-235).

The PCR process is well known in the art and include, for example, reverse transcription PCR, ligation mediated PCR, digital PCR (dPCR), or droplet digital PCR (ddPCR). For a review of PCR methods and protocols, see, e.g., Innis et al, eds., PCR Protocols, A Guide to Methods and Application, Academic Press, Inc., San Diego, Calif 1990; U.S. Pat. No. 4,683,202 (Mullis). PCR reagents and protocols are also available from commercial vendors, such as Roche Molecular Systems. In some instances, PCR is carried out as an automated process with a thermostable enzyme. In this process, the temperature of the reaction mixture is cycled through a denaturing region, a primer annealing region, and an extension reaction region automatically. Machines specifically adapted for this purpose are commercially available.

In some embodiments, amplified sequences are also measured using invasive cleavage reactions such as the Invader® technology (Zou et al, 2010, Association of Clinical Chemistry (AACC) poster presentation on Jul. 28, 2010, “Sensitive Quantification of Methylated Markers with a Novel Methylation Specific Technology; and U.S. Pat. No. 7,011,944 (Prudent et al)).

Suitable next generation sequencing technologies are widely available. Examples include the 454 Life Sciences platform (Roche, Branford, CT) (Margulies et al. 2005 Nature, 437, 376-380); Illumina's Genome Analyzer, GoldenGate Methylation Assay, or Infinium Methylation Assays, i.e., Infinium HumanMethylation 27K BeadArray or VeraCode GoldenGate methylation array (Illumina, San Diego, CA; Bibkova et al, 2006, Genome Res. 16, 383-393; U.S. Pat. Nos. 6,306,597 and 7,598,035 (Macevicz); U.S. Pat. No. 7,232,656 (Balasubramanian et al.)); QX200™ Droplet Digital™ PCR System from Bio-Rad; or DNA Sequencing by Ligation, SOLiD System (Applied Biosystems/Life Technologies; U.S. Pat. Nos. 6,797,470, 7,083,917, 7,166,434, 7,320,865, 7,332,285, 7,364,858, and 7,429,453 (Barany et al); the Helicos True Single Molecule DNA sequencing technology (Harris et al, 2008 Science, 320, 106-109; U.S. Pat. Nos. 7,037,687 and 7,645,596 (Williams et al); 7, 169,560 (Lapidus et al); U.S. Pat. No. 7,769,400 (Harris)), the single molecule, real-time (SMRT™) technology of Pacific Biosciences, and sequencing (Soni and Meller, 2007, Clin. Chem. 53, 1996-2001); semiconductor sequencing (Ion Torrent; Personal Genome Machine); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g., Oxford Nanopore, Genia Technologies, and Nabsys). These systems allow the sequencing of many nucleic acid molecules isolated from a specimen at high orders of multiplexing in a parallel fashion. Each of these platforms allow sequencing of clonally expanded or non-amplified single molecules of nucleic acid fragments. Certain platforms involve, for example, (i) sequencing by ligation of dye-modified probes (including cyclic ligation and cleavage), (ii) pyrosequencing, and (iii) single-molecule sequencing.

Pyrosequencing is a nucleic acid sequencing method based on sequencing by synthesis, which relies on detection of a pyrophosphate released on nucleotide incorporation. Generally, sequencing by synthesis involves synthesizing, one nucleotide at a time, a DNA strand complimentary to the strand whose sequence is being sought. Study nucleic acids may be immobilized to a solid support, hybridized with a sequencing primer, incubated with DNA polymerase, ATP sulfurylase, luciferase, apyrase, adenosine 5′ phosphsulfate and luciferin. Nucleotide solutions are sequentially added and removed. Correct incorporation of a nucleotide releases a pyrophosphate, which interacts with ATP sulfurylase and produces ATP in the presence of adenosine 5′ phosphsulfate, fueling the luciferin reaction, which produces a chemiluminescent signal allowing sequence determination. Machines for pyrosequencing and methylation specific reagents are available from Qiagen, Inc. (Valencia, CA). See also Tost and Gut, 2007, Nat. Prot. 2 2265-2275. An example of a system that can be used by a person of ordinary skill based on pyrosequencing generally involves the following steps: ligating an adaptor nucleic acid to a study nucleic acid and hybridizing the study nucleic acid to a bead; amplifying a nucleotide sequence in the study nucleic acid in an emulsion; sorting beads using a picoliter multiwell solid support; and sequencing amplified nucleotide sequences by pyrosequencing methodology (e.g., Nakano et al, 2003, J. Biotech. 102, 117-124). Such a system can be used to exponentially amplify amplification products generated by a process described herein, e.g., by ligating a heterologous nucleic acid to the first amplification product generated by a process described herein.

### CpG Methylation Data Analysis Methods

In certain embodiments, the methylation values measured for biomarkers of a biomarker panel are mathematically combined and the combined value is correlated to the underlying diagnostic question. In some instances, methylated biomarker values are combined by any appropriate state of the art mathematical method. Well-known mathematical methods for correlating a biomarker combination to a disease status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate an epigenetic marker or biomarker combination described herein. In one embodiment, the method used in a correlating methylation status of an epigenetic marker or biomarker combination, e.g. to diagnose liver cancer or a liver cancer subtype, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. O., Pattern Classification, Wiley Interscience, 2nd Edition (2001).

In one embodiment, the correlated results for each methylation panel are rated by their correlation to the disease or tumor type positive state, such as for example, by p-value test or t-value test or F-test. Rated (best first, i.e. low p- or t-value) biomarkers are then subsequently selected and added to the methylation panel until a certain diagnostic value is reached. Such methods include identification of methylation panels, or more broadly, genes that were differentially methylated among several classes using, for example, a random-variance t-test (Wright G. W. and Simon R, Bioinformatics 19:2448-2455, 2003). Other methods include the step of specifying a significance level to be used for determining the epigenetic markers that will be included in the biomarker panel. Epigenetic markers that are differentially methylated between the classes at a univariate parametric significance level less than the specified threshold are included in the panel. It doesn't matter whether the specified significance level is small enough to exclude enough false discoveries. In some problems better prediction is achieved by being more liberal about the biomarker panels used as features. In some cases, the panels are biologically interpretable and clinically applicable, however, if fewer markers are included. Similar to cross-validation, biomarker selection is repeated for each training set created in the cross-validation process. That is for the purpose of providing an unbiased estimate of prediction error. The methylation panel for use with new patient sample data is the one resulting from application of the methylation selection and classifier of the “known” methylation information, or control methylation panel.

Models for utilizing methylation profile to predict the class of future samples can also be used. These models may be based on the Compound Covariate Predictor (Radmacher et al. Journal of Computational Biology 9:505-511, 2002), Diagonal Linear Discriminant Analysis (Dudoit et al. Journal of the American Statistical Association 97:77-87, 2002), Nearest Neighbor Classification (also Dudoit et al.), and Support Vector Machines with linear kernel (Ramaswamy et al. PNAS USA 98:15149-54, 2001). The models incorporated markers that were differentially methylated at a given significance level (e.g. 0.01, 0.05 or 0.1) as assessed by the random variance t-test (Wright G. W. and Simon R. Bioinformatics 19:2448-2455, 2003). The prediction error of each model using cross validation, preferably leave-one-out cross-validation (Simon et al. Journal of the National Cancer Institute 95:14-18, 2003 can be estimated. For each leave-one-out cross-validation training set, the entire model building process is repeated, including the epigenetic marker selection process. In some instances, it is also evaluated in whether the cross-validated error rate estimate for a model is significantly less than one would expect from random prediction. In some cases, the class labels are randomly permuted and the entire leave-one-out cross-validation process is then repeated. The significance level is the proportion of the random permutations that gives a cross-validated error rate no greater than the cross-validated error rate obtained with the real methylation data.

Another classification method is the greedy-pairs method described by Bo and Jonassen (Genome Biology 3(4):research0017.1-0017.11, 2002). The greedy-pairs approach starts with ranking all markers based on their individual t-scores on the training set. This method attempts to select pairs of markers that work well together to discriminate the classes.

Furthermore, a binary tree classifier for utilizing methylation profile is optionally used to predict the class of future samples. The first node of the tree incorporated a binary classifier that distinguished two subsets of the total set of classes. The individual binary classifiers are based on the “Support Vector Machines” incorporating markers that were differentially expressed among markers at the significance level (e.g. 0.01, 0.05 or 0.1) as assessed by the random variance t-test (Wright G. W. and Simon R. Bioinformatics 19:2448-2455, 2003). Classifiers for all possible binary partitions are evaluated and the partition selected is that for which the cross-validated prediction error is minimum. The process is then repeated successively for the two subsets of classes determined by the previous binary split. The prediction error of the binary tree classifier can be estimated by cross-validating the entire tree building process. This overall cross-validation includes re-selection of the optimal partitions at each node and re-selection of the markers used for each cross-validated training set as described by Simon et al. (Simon et al. Journal of the National Cancer Institute 95:14-18, 2003). Several-fold cross validation in which a fraction of the samples is withheld, a binary tree developed on the remaining samples, and then class membership is predicted for the samples withheld. This is repeated several times, each time withholding a different percentage of the samples. The samples are randomly partitioned into fractional test sets (Simon R and Lam A. BRB-ArrayTools User Guide, version 3.2. Biometric Research Branch, National Cancer Institute).

Thus, in one embodiment, the correlated results for each marker b) are rated by their correct correlation to the disease, preferably by p-value test. It is also possible to include a step in that the markers are selected d) in order of their rating.

In additional embodiments, factors such as the value, level, feature, characteristic, property, etc. of a transcription rate, mRNA level, translation rate, protein level, biological activity, cellular characteristic or property, genotype, phenotype, etc. can be utilized in addition prior to, during, or after administering a therapy to a patient to enable further analysis of the patient's cancer status.

In some embodiments, a diagnostic test to correctly predict status is measured as the sensitivity of the assay, the specificity of the assay or the area under a receiver operated characteristic (“ROC”) curve. In some instances, sensitivity is the percentage of true positives that are predicted by a test to be positive, while specificity is the percentage of true negatives that are predicted by a test to be negative. In some cases, an ROC curve provides the sensitivity of a test as a function of 1-specificity. The greater the area under the ROC curve, for example, the more accurate or powerful the predictive value of the test. Other useful measures of the utility of a test include positive predictive value and negative predictive value. Positive predictive value is the percentage of people who test positive that are actually positive. Negative predictive value is the percentage of people who test negative that are actually negative.

In some embodiments, one or more of the biomarkers disclosed herein show a statistical difference in different samples of at least p<0.05, p<10⁻², p<10⁻³, p<10⁻⁴ or p<10⁻⁵. Diagnostic tests that use these biomarkers may show an ROC of at least 0.6, at least about 0.7, at least about 0.8, or at least about 0.9. In some instances, the biomarkers are differentially methylated in different subjects with or without liver cancer. In additional instances, the biomarkers for different subtypes of liver cancer are differentially methylated. In certain embodiments, the biomarkers are measured in a patient sample using the methods described herein and compared, for example, to predefined biomarker levels and are used to determine whether the patient has liver cancer, which liver cancer subtype does the patient have, and/or what is the prognosis of the patient having liver cancer. In other embodiments, the correlation of a combination of biomarkers in a patient sample is compared, for example, to a predefined set of biomarkers. In some embodiments, the measurement(s) is then compared with a relevant diagnostic amount(s), cut-off(s), or multivariate model scores that distinguish between the presence or absence of liver cancer, between liver cancer subtypes, and between a “good” or a “poor” prognosis. As is well understood in the art, by adjusting the particular diagnostic cut-off(s) used in an assay, one can increase sensitivity or specificity of the diagnostic assay depending on the preference of the diagnostician. In some embodiments, the particular diagnostic cut-off is determined, for example, by measuring the amount of biomarker hypermethylation or hypomethylation in a statistically significant number of samples from patients with or without liver cancer and from patients with different liver cancer subtypes, and drawing the cut-off to suit the desired levels of specificity and sensitivity.

### Kits/Article of Manufacture

In some embodiments, provided herein include kits for detecting and/or characterizing the methylation profile of a biomarker described herein. In some instances, the kit comprises a plurality of primers or probes to detect or measure the methylation status/levels of one or more samples. Such kits comprise, in some instances, at least one polynucleotide that hybridizes to at least one of the methylation marker sequences described herein and at least one reagent for detection of gene methylation. Reagents for detection of methylation include, e.g., sodium bisulfate, polynucleotides designed to hybridize to sequence that is the product of a marker sequence if the marker sequence is not methylated (e.g., containing at least one C-U conversion), and/or a methylation-sensitive or methylation-dependent restriction enzyme. In some cases, the kits provide solid supports in the form of an assay apparatus that is adapted to use in the assay. In some instances, the kits further comprise detectable labels, optionally linked to a polynucleotide, e.g., a probe, in the kit.

In some embodiments, the kits comprise one or more (e.g., 1, 2, 3, 4, or more) different polynucleotides (e.g., primers and/or probes) capable of specifically amplifying at least a portion of a DNA region of a biomarker described herein. Optionally, one or more detectably-labeled polypeptides capable of hybridizing to the amplified portion are also included in the kit. In some embodiments, the kits comprise sufficient primers to amplify 2, 3, 4, 5, 6, 7, 8, 9, 10, or more different DNA regions or portions thereof, and optionally include detectably-labeled polynucleotides capable of hybridizing to each amplified DNA region or portion thereof. The kits further can comprise a methylation-dependent or methylation sensitive restriction enzyme and/or sodium bisulfite.

In some embodiments, the kits comprise sodium bisulfite, primers and adapters (e.g., oligonucleotides that can be ligated or otherwise linked to genomic fragments) for whole genome amplification, and polynucleotides (e.g., detectably-labeled polynucleotides) to quantify the presence of the converted methylated and or the converted unmethylated sequence of at least one cytosine from a DNA region of an epigenetic marker described herein.

In some embodiments, the kits comprise methylation sensing restriction enzymes (e.g., a methylation-dependent restriction enzyme and/or a methylation-sensitive restriction enzyme), primers and adapters for whole genome amplification, and polynucleotides to quantify the number of copies of at least a portion of a DNA region of an epigenetic marker described herein.

In some embodiments, the kits comprise a methylation binding moiety and one or more polynucleotides to quantify the number of copies of at least a portion of a DNA region of a marker described herein. A methylation binding moiety refers to a molecule (e.g., a polypeptide) that specifically binds to methyl-cytosine.

Examples include restriction enzymes or fragments thereof that lack DNA cutting activity but retain the ability to bind methylated DNA, antibodies that specifically bind to methylated DNA, etc.).

In some embodiments, the kit includes a packaging material. As used herein, the term “packaging material” can refer to a physical structure housing the components of the kit. In some instances, the packaging material maintains sterility of the kit components, and is made of material commonly used for such purposes (e.g., paper, corrugated fiber, glass, plastic, foil, ampules, etc.). Other materials useful in the performance of the assays are included in the kits, including test tubes, transfer pipettes, and the like. In some cases, the kits also include written instructions for the use of one or more of these reagents in any of the assays described herein.

In some embodiments, kits also include a buffering agent, a preservative, or a protein/nucleic acid stabilizing agent. In some cases, kits also include other components of a reaction mixture as described herein. For example, kits include one or more aliquots of thermostable DNA polymerase as described herein, and/or one or more aliquots of dNTPs. In some cases, kits also include control samples of known amounts of template DNA molecules harboring the individual alleles of a locus. In some embodiments, the kit includes a negative control sample, e.g., a sample that does not contain DNA molecules harboring the individual alleles of a locus. In some embodiments, the kit includes a positive control sample, e.g., a sample containing known amounts of one or more of the individual alleles of a locus.

### Exemplary Profiles and Uses

In some aspects, provided herein is a method of generating a biomarker profile from a sample obtained from an individual, wherein the biomarker profile comprises a methylation profile comprising data of one or more CpG sites from Table 11, the method comprising: (a) determining a methylation status for each of the one or more CpG sites of the methylation profile from a treated genomic DNA derived from the sample; and (b) generating the methylation profile based on the methylation status of the one or more CpG site of the methylation profile to generate the biomarker profile.

In some embodiments, the one or more CpG sites of the methylation profile comprises one or more CpG sites of one or more of the following genes: PSD4, EVL, RASSF5, MAP3K8, LAT2, HEXDC, MYO1G, CTTN, UBE4B, KIAA0930, LTA, C16orf54, LOC101928253, URI1, TNFAIP8L2 (SCNM1), FOXP4 (AS1), IFITM1, RPS6KA1, LINC01298, HIST1H4F, BDH1, MIR153-2, PFN3, LOC101929153, MIR1302-7, LOC100506585, DIRAS1, or MIR21.

In some embodiments, the one or more CpG sites of the methylation profile comprises one or more CpG sites of the following genes: PSD4, EVL, RASSF5, MAP3K8, LAT2, HEXDC, MYO1G, CTTN, UBE4B, KIAA0930, LTA, C16orf54, LOC101928253, URI1, TNFAIP8L2 (SCNM1), FOXP4 (AS1), IFITM1, RPS6KA1, LINC01298, HIST1H4F, BDH1, MIR153-2, PFN3, LOC101929153, MIR1302-7, LOC100506585, DIRAS1, and MIR21.

In some embodiments, the one or more CpG sites of the methylation profile comprises one or more of the following CpG sites: chr17:57915773-57915774, chr19:2723147-2723148, chr19:2723034-2723035, chr17:57915717-57915718, chr5:4629212-4629213, chr5:4629193-4629194, chr19:2723181-2723182, chr19:2723169-2723170, chr6:26240930-26240931, chr6:26240920-26240921, chr19:30562385-30562386, chr19:30562320-30562321, chr11:314074-314075, chr6:26240975-26240976, chr6:26240950-26240951, chr6:26240939-26240940, chr19:2723189-2723190, or chr19:2723184-2723185.

In some embodiments, the one or more CpG sites of the methylation profile comprises the following CpG sites: chr17:57915773-57915774, chr19:2723147-2723148, chr19:2723034-2723035, chr17:57915717-57915718, chr5:4629212-4629213, chr5:4629193-4629194, chr19:2723181-2723182, chr19:2723169-2723170, chr6:26240930-26240931, chr6:26240920-26240921, chr19:30562385-30562386, chr19:30562320-30562321, chr11:314074-314075, chr6:26240975-26240976, chr6:26240950-26240951, chr6:26240939-26240940, chr19:2723189-2723190, and chr19:2723184-2723185.

In some embodiments, the one or more CpG sites of the methylation profile comprises the following CpG sites: chr17:57915773-57915774, chr19:2723147-2723148, chr19:2723034-2723035, chr17:57915717-57915718, chr5:4629212-4629213, chr5:4629193-4629194, chr19:2723181-2723182, chr19:2723169-2723170, chr6:26240930-26240931, chr6:26240920-26240921, chr19:30562385-30562386, chr19:30562320-30562321, chr11:314074-314075, chr6:26240975-26240976, chr6:26240950-26240951, chr6:26240939-26240940, chr19:2723189-2723190, chr19:2723184-2723185, chr8:142852883-142852884, chr8:142852876-142852877, chr7:157563602-157563603, chr11:314113-314114, chr11:314106-314107, chr11:314098-314099, chr11:314086-314087, chr1:206753453-206753454, chr7:157319206-157319207, chr7:157319203-157319204, chr7:157319199-157319200, chr1:151129298-151129299, chr7:73641105-73641106, chr7:73641071-73641072, chr16:29757375-29757376, chr16:29757360-29757361, chr11:70211540-70211541, chr11:70211534-70211535, chr11:70211531-70211532, chr11:70211523-70211524, chr14:100532797-100532798, chr14:100532790-100532791, chr5:176829777-176829778, chr5:176829755-176829756, chr16:29757350-29757351, chr16:29757323-29757324, chr3:197283111-197283112, chr6:11976066-11976067, chr6:11976024-11976025, chr6:41528502-41528503, chr6:41528499-41528500, chr6:41528497-41528498, chr6:41528491-41528492, chr16:29757344-29757345, chr16:29757334-29757335, chr17:80358932-80358933, chr17:80358919-80358920, chr6:31527920-31527921, chr6:31527893-31527894, chr6:31527889-31527890, chr2:113931525-113931526, chr2:113931518-113931519, chr7:45018849-45018850, chr8:96193941-96193942, chr8:96193898-96193899, chr1:26872538-26872539, chr1:26872525-26872526, chr1:26872518-26872519, chr22:45631384-45631385, chr22:45631379-45631380, chr10:30818618-30818619, chr10:30818611-30818612, chr10:30818609-30818610, chr1:10134620-10134621, chr1:10134610-10134611, chr17:80358850-80358851, chr17:80358847-80358848, chr17:80358829-80358830, and chr17:80358819-80358820.

In some embodiments, the one or more CpG sites of the methylation profile comprises the following CpG sites: chr17:57915773-57915774, chr19:2723034-2723035, chr5:4629193-4629194, chr6:26240920-26240921, chr19:30562320-30562321, chr11:314074-314075, chr8:142852876-142852877, chr7:157563602-157563603, chr1:206753453-206753454, and chr7:157319199-157319200. In some embodiments, the one or more CpG sites of the methylation profile comprises the following CpG sites: chr17:57915773-57915774, chr19:2723034-2723035, chr5:4629193-4629194, chr6:26240920-26240921, and chr19:30562320-30562321. In some embodiments, the one or more CpG sites of the methylation profile comprises the following CpG sites: chr11:314074-314075, chr8:142852876-142852877, chr7:157563602-157563603, chr1:206753453-206753454, and chr7:157319199-157319200. In some embodiments, the one or more CpG sites of the methylation profile comprises the following CpG sites: chr17:57915773-57915774, chr5:4629193-4629194, chr19:30562320-30562321, chr8:142852876-142852877, and chr1:206753453-206753454. In some embodiments, the one or more CpG sites of the methylation profile comprises the following CpG sites: chr19:2723034-2723035, chr6:26240920-26240921, chr11:314074-314075, chr7:157563602-157563603, and chr7:157319199-157319200. In some embodiments, the one or more CpG sites of the methylation profile comprises the following CpG sites: chr17:57915773-57915774, chr6:26240920-26240921, chr8:142852876-142852877, chr7:157563602-157563603, and chr1:206753453-206753454. In some embodiments, the one or more CpG sites of the methylation profile comprises the following CpG sites: chr19:2723034-2723035, chr5:4629193-4629194, chr19:30562320-30562321, chr11:314074-314075, and chr7:157319199-157319200.

In some embodiments, the methylation status of each CpG site is based on a p-value, and wherein the 0-value of a CpG site is determined based on the proportion of instances of methylation at the CpG site divided by the sum of the instances of methylation at the CpG site plus the instances where the CpG site is not methylated.

In some embodiments, the methylation status is determined using sequencing information derived from the treated genomic DNA. In some embodiments, the sequencing information is obtained using a sequencing technique. In some embodiments, the sequencing technique is a next generation sequencing technique. In some embodiments, the sequencing technique is a whole-genome sequencing technique. In some embodiments, the sequencing technique is a targeted sequencing technique. In some embodiments, the sequence technique is capable of providing paired-end sequencing reads. In some embodiments, the sequencing technique is performed such that the sequencing depth is at least about 50×.

In some embodiments, the method further comprises performing the sequencing technique.

In some embodiments, the method further comprises obtaining the treated genomic DNA derived from the sample. In some embodiments, the obtaining the treated genomic DNA comprises subjecting DNA derived from the sample to processing that enables determination of a methylation status of a CpG. In some embodiments, the processing to obtain the treated genomic DNA comprises an enzyme-based technique for the conversion of unmethylated cytosines to enable the determination of the methylation status of a CpG site. In some embodiments, the enzyme-based technique is an EM-seq technique. In some embodiments, the processing to obtain the treated genomic DNA comprises a bisulfite-based technique.

In some embodiments, the detecting the methylation status for each of the one or more CpG sites is based on sequence reads obtained from the treated genomic DNA.

In some embodiments, the sequence reads used for the detecting the methylation status for each of the one or more CpG sites are pre-processed. In some embodiments, the sequence read pre-processing comprises removing low-quality reads. In some embodiments, the sequence read pre-processing comprises removing sequence adaptor sequences. In some embodiments, the sequence read pre-processing comprises removing M-bias. In some embodiments, the sequence read pre-processing comprises producing paired reads. In some embodiments, the sequence read pre-processing comprises removing sequence reads having a sequencing depth of less than 50×. In some embodiments, the sequence read pre-processing comprises mapping sequence reads to a reference genome. In some embodiments, the reference genome is a human reference genome.

In some embodiments, the biomarker profile further comprises a polypeptide profile. In some embodiments, the polypeptide profile comprises data of one or more of an alpha fetoprotein (AFP) level, a Lens culinaris agglutinin-reactive AFP (AFP-L3%) level, or a des-gamma-carboxyprothrombin (DCP) level obtained from the individual. In some embodiments, the polypeptide profile comprises data of the AFP level, AFP-L3%, and the DCP level. In some embodiments, the AFP level, AFP-L3%, and DCP level are based on respective serum concentrations measured from the individual. In some embodiments, the serum concentrations are derived from the sample obtained from the individual.

In some embodiments, the biomarker profile further comprises a demographic profile. In some embodiments, the demographic profile comprises the age of the individual. In some embodiments, the demographic profile comprises the sex of the individual.

In other aspects, provided herein is a method of generating a biomarker profile from a sample obtained from an individual, wherein the biomarker profile comprises: a methylation profile comprising data of one or more CpG sites from Table 11; a polypeptide profile comprising data of one or more of an AFP level, an AFP-L3%, or a DCP level; and a demographic profile comprising data of one or more of the age or sex of the individual, the method comprising: (a) determining, for the methylation profile, a methylation status for each of the one or more CpG sites of the methylation profile from a treated genomic DNA derived from the sample; (b) determining, for the polypeptide profile, one or more the AFP level, the AFP-L3%, or the DCP level from the sample; (c) determining, for the demographic profile, one or more of the age or sex of the individual; and (d) generating the biomarker profile based on the methylation profile, the polypeptide profile, and the demographic profile. In some embodiments, the methylation profile comprises data of all CpG sites from Table 11. In some embodiments, the polypeptide profile comprises the AFP level, the AFP-L3%, and the DCP level. In some embodiments, the demographic profile comprises the age and sex of the individual.

In some embodiments, the generating the biomarker profile comprises providing the methylation profile, the polypeptide profile, and/or the demographic profile to one or more machine learning classifiers to generate the biomarker profile. In some embodiments, the one or more machine learning classifiers comprises a random forest model. In some embodiments, the one or more machine learning classifiers comprises a grid-search technique. In some embodiments, the grid-search technique comprises optimizing the hyper parameters of the random forest model.

In some embodiments, the biomarker profile combines the methylation profile, the polypeptide profile, and/or the demographic profile using a decision tree model.

In some embodiments, at least one of the one or more machine learning classifiers is trained using a data derived from one or more individuals having known condition(s) and one or more associated methylation profiles, polypeptide profiles, or demographic profiles. In some embodiments, the known condition is whether the individual has a liver cancer or chronic liver disease.

In some embodiments, the sample is a liquid biopsy sample. In some embodiments, the sample is a blood sample. In some embodiments, the sample comprises cfDNA. In some embodiments, the sample is a cfDNA sample.

In some embodiments, the subject is suspected of having a liver cancer. In some embodiments, the liver cancer is hepatocellular carcinoma (HCC). In some embodiments, the HCC is early stage HCC. In some embodiments, the early stage HCC is AJCC stage I and/or stage II HCC.

In other aspects, provided is a system for determining a biomarker profile from a sample obtained from an individual, wherein the biomarker profile comprises one or more of: a methylation profile comprising data of one or more CpG sites from Table 11; a polypeptide profile comprising data of one or more of an AFP level, an AFP-L3%, or a DCP level; or a demographic profile comprising data of one or more of the age or sex of the individual, the system comprising: one or more processors; and memory storing one or more programs, the one or more programs configured to be executed by the one or more processors, the one or more programs including instructions for: receiving sequencing information comprising sequence reads; determining one or more of the following: the methylation profile based on data of the one or more CpG sites from Table 11; the polypeptide profile based on data of the one or more of the AFP level, the AFP-L3%, or the DCP level; or the demographic profile based on data of the one or more of the age or sex of the individual, determining the biomarker profile based on one or more of the methylation profile, the polypeptide profile, or the demographic profile.

In some embodiments, the system further comprises one or more machine learning classifiers configured to determine the biomarker profile.

In other aspects, provided is a system for determining a biomarker profile from a sample obtained from an individual, wherein the biomarker profile comprises one or more of: a methylation profile comprising data of one or more CpG sites from Table 11; a polypeptide profile comprising data of one or more of an AFP level, an AFP-L3%, or a DCP level; or a demographic profile comprising data of one or more of the age or sex of the individual, the system comprising: one or more processors; and memory storing one or more programs, the one or more programs configured to be executed by the one or more processors, the one or more programs including instructions for: receiving data pertaining to one or more of the methylation profile, the polypeptide profile, and the demographic profile; applying one or more machine learning classifiers to the received data to determine the biomarker profile based on one or more of the methylation profile, the polypeptide profile, or the demographic profile. In some embodiments, the one or more machine learning classifiers comprises a random forest model. In some embodiments, the one or more machine learning classifiers comprises a grid-search technique. In some embodiments, the grid-search technique comprises optimizing the hyper parameters of the random forest model. In some embodiments, the biomarker profile combines the methylation profile, the polypeptide profile, and/or the demographic profile using a decision tree model. In some embodiments, at least one of the one or more machine learning classifiers is trained using a data derived from one or more individuals having known condition(s) and one or more associated methylation profiles, polypeptide profiles, or demographic profiles. In some embodiments, the known condition is whether the individual has a liver cancer or chronic liver disease.

In other aspects, provided herein is a kit for generating a biomarker profile from a sample from an individual, the kit comprising one or more probes, wherein each probe is suitable for detecting a methylation status of a CpG site in Table 11. In some embodiments, each probe hybridizes to at least a portion of the targeted region in Table 11. In some embodiments, the at least the portion is at least about 50 base pairs. In some embodiments, the at least the portion is about 120 base pairs. In some embodiments, the each probe is complementary to the target portion. In some embodiments, each probe is about 50 to about 120 base pairs. In some embodiments, the terminal end of a probe overlaps, e.g., by at least two base pairs, with a CpG site on a target nucleic acid. In some embodiments, each probe is configured to determine the methylation status of one or more CpG sties from Table 11.

In some embodiments, the kit further comprises reagents to determine one or more of an AFP level, an AFP-L3%, or a DCP level from a sample from the individual.

In some embodiments, the kit further comprises instructions for determining the age and/or sex of the individual.

In other aspects, provided herein is a method of diagnosing an individual as having a liver cancer, e.g., hepatocellular carcinoma, including early stage (such as stage I or stage II as established by AJCC), based on generating a biomarker profile from a sample obtained from an individual, wherein the biomarker profile comprises one or more of: a methylation profile comprising data of one or more CpG sites from Table 11; a polypeptide profile comprising data of one or more of an AFP level, an AFP-L3%, or a DCP level; and a demographic profile comprising data of one or more of the age or sex of the individual, the method comprising: determining, for the methylation profile, as necessary, a methylation status for each of the one or more CpG sites of the methylation profile from a treated genomic DNA derived from the sample; (b) determining, for the polypeptide profile, as necessary, one or more the AFP level, the AFP-L3%, or the DCP level from the sample; (c) determining, for the demographic profile, as necessary, one or more of the age or sex of the individual; and (d) generating the biomarker profile based on the one or more of the methylation profile, the polypeptide profile, or the demographic profile.

In other aspects, provided herein is a method of diagnosing an individual as having a liver cancer, e.g., hepatocellular carcinoma, including early stage (such as stage I or stage II as established by AJCC), based on generating a biomarker profile from a sample obtained from an individual, wherein the biomarker profile comprises: a methylation profile comprising data of one or more CpG sites from Table 11; a polypeptide profile comprising data of one or more of an AFP level, an AFP-L3%, or a DCP level; and a demographic profile comprising data of one or more of the age or sex of the individual, the method comprising: (a) determining, for the methylation profile, a methylation status for each of the one or more CpG sites of the methylation profile from a treated genomic DNA derived from the sample; (b) determining, for the polypeptide profile, one or more the AFP level, the AFP-L3%, or the DCP level from the sample; (c) determining, for the demographic profile, one or more of the age or sex of the individual; and (d) generating the biomarker profile based on the methylation profile, the polypeptide profile, and the demographic profile. In some embodiments, the methylation profile comprises data of all CpG sites from Table 11. In some embodiments, the polypeptide profile comprises the AFP level, the AFP-L3%, and the DCP level. In some embodiments, the demographic profile comprises the age and sex of the individual.

Table 11 is provided below. In Table 11, the CpG site ID refers to a chromosome start and end site that corresponds to a particular CpG site. The third column, CpG ID, is reported in accordance with the hg19 genome reference.

TABLE 11
CpG markers useful for the description provided herein.

| | | CpG ID | Probe ID | |
|---|---|---|---|---|
| CpG | | (chr:start- | (chr:start- | Exemplary sequence of the |
| site ID | Gene | end) | end) | region targeted by the probe |
| chr17_ | MIR21 | chr17: | chr17: | CTGTCCCCGGCATAGGTCCATCTCTGCAG |
| 57915773_ | | 57915773- | 57915657- | AAGCCATTTCAGGAGTACCTGGAGGCTC |
| 57915774 | | 57915774 | 57915777 | AACGGCAGAAGCTTCACCACAAAAGCGA |
| | | | | AATGGGCACACCACAGGTAAGACTTTAA |
| | | | | TCCGGTT |
| chr19_ | DIRAS1 | chr19: | chr19: | CGCCTGTAGGGTTGCTGGGAACACATGG |
| 2723147_ | | 2723147- | 2723033- | GCAGGGCGCCCACCTACACGGGGCTGCT |
| 2723148 | | 2723148 | 2723261 | GGGCACCCAAGCACTCAGACGGCAGCCC |
| | | | | TGCCCCTTCCCAGTCCGCCTGCCTGCCTT |
| | | | | CGTCTCCTGAGTGACCCACCAGCGTGGG |
| | | | | GCAGGGCGTCGGGCCGGCTCCCTGTCTC |
| | | | | CTCCGGGCACCAGCTCAGGCCGGGGCAG |
| | | | | GCTGACTCATGCGTAGGAGAGTGGGCAG |
| | | | | GGC |
| chr19_ | DIRAS1 | chr19: | chr19: | CGCCTGTAGGGTTGCTGGGAACACATGG |
| 2723034_ | | 2723034- | 2723033- | GCAGGGCGCCCACCTACACGGGGCTGCT |
| 2723035 | | 2723035 | 2723261 | GGGCACCCAAGCACTCAGACGGCAGCCC |
| | | | | TGCCCCTTCCCAGTCCGCCTGCCTGCCTT |
| | | | | CGTCTCCTGAGTGACCCACCAGCGTGGG |
| | | | | GCAGGGCGTCGGGCCGGCTCCCTGTCTC |
| | | | | CTCCGGGCACCAGCTCAGGCCGGGGCAG |
| | | | | GCTGACTCATGCGTAGGAGAGTGGGCAG |
| | | | | GGC |
| chr17_ | MIR21 | chr17: | chr17: | CTGTCCCCGGCATAGGTCCATCTCTGCAG |
| 57915717_ | | 57915717- | 57915657- | AAGCCATTTCAGGAGTACCTGGAGGCTC |
| 57915718 | | 57915718 | 57915777 | AACGGCAGAAGCTTCACCACAAAAGCGA |
| | | | | AATGGGCACACCACAGGTAAGACTTTAA |
| | | | | TCCGGTT |
| chr5_ | LOC101 | chr5: | chr5: | CGGTTCCAAGCTGAGCCTAACTGGGGAA |
| 4629212_ | 929153 | 4629212- | 4629157- | ATCCAGACGGACATTTCCCCAGGAAACG |
| 4629213 | | 4629213 | 4629277 | GGCTGCCTTCCCCTCCTGGATCTCCTCCA |
| | | | | TGACAGGGACTCTGAAGAGCTGTGCCAA |
| | | | | CTCAGGC |
| chr5_ | LOC101 | chr5: | chr5: | CGGTTCCAAGCTGAGCCTAACTGGGGAA |
| 4629193_ | 929153 | 4629193- | 4629157- | ATCCAGACGGACATTTCCCCAGGAAACG |
| 4629194 | | 4629194 | 4629277 | GGCTGCCTTCCCCTCCTGGATCTCCTCCA |
| | | | | TGACAGGGACTCTGAAGAGCTGTGCCAA |
| | | | | CTCAGGC |
| chr19_ | DIRAS1 | chr19: | chr19: | CGCCTGTAGGGTTGCTGGGAACACATGG |
| 2723181_ | | 2723181- | 2723033- | GCAGGGCGCCCACCTACACGGGGCTGCT |
| 2723182 | | 2723182 | 2723261 | GGGCACCCAAGCACTCAGACGGCAGCCC |
| | | | | TGCCCCTTCCCAGTCCGCCTGCCTGCCTT |
| | | | | CGTCTCCTGAGTGACCCACCAGCGTGGG |
| | | | | GCAGGGCGTCGGGCCGGCTCCCTGTCTC |
| | | | | CTCCGGGCACCAGCTCAGGCCGGGGCAG |
| | | | | GCTGACTCATGCGTAGGAGAGTGGGCAG |
| | | | | GGC |
| chr19_ | DIRAS1 | chr19: | chr19: | CGCCTGTAGGGTTGCTGGGAACACATGG |
| 2723169_ | | 2723169- | 2723033- | GCAGGGCGCCCACCTACACGGGGCTGCT |
| 2723170 | | 2723170 | 2723261 | GGGCACCCAAGCACTCAGACGGCAGCCC |
| | | | | TGCCCCTTCCCAGTCCGCCTGCCTGCCTT |
| | | | | CGTCTCCTGAGTGACCCACCAGCGTGGG |
| | | | | GCAGGGCGTCGGGCCGGCTCCCTGTCTC |
| | | | | CTCCGGGCACCAGCTCAGGCCGGGGCAG |
| | | | | GCTGACTCATGCGTAGGAGAGTGGGCAG |
| | | | | GGC |
| chr6_ | HIST1H4F | chr6: | chr6: | GCCGTAACCTACACGGAGCACGCCAAGC |
| 26240930_ | | 26240930- | 26240860- | GTAAGACAGTCACTGCAATGGATGTTGT |
| 26240931 | | 26240931 | 26240980 | CTACGCGCTCAAGCGCCAGGGACGCACT |
| | | | | CTGTACGGCTTTGGTGGCTGAGCCTCACC |
| | | | | CCGGCTT |
| chr6_ | HIST1H4F | chr6: | chr6: | GCCGTAACCTACACGGAGCACGCCAAGC |
| 26240920_ | | 26240920- | 26240860- | GTAAGACAGTCACTGCAATGGATGTTGT |
| 26240921 | | 26240921 | 26240980 | CTACGCGCTCAAGCGCCAGGGACGCACT |
| | | | | CTGTACGGCTTTGGTGGCTGAGCCTCACC |
| | | | | CCGGCTT |
| chr19_ | URI1 | chr19: | chr19: | CGGCTGCTAGAGGGGAGGGAGGGGATCT |
| 30562385_ | | 30562385- | 30562250- | CGTGCCCCCGATCTGGCACCGGGGTGGG |
| 30562386 | | 30562386 | 30562681 | CAGGGCATATGGACGGCAGCCATTGGCG |
| | | | | AGGTGCCCACACCAGGCCCTGGCTTCGG |
| | | | | GCCCAGCATGAGCCTGGGCCGGCGGTGG |
| | | | | GTAAGCTCTCTATCCCTCTCTGCCCTATA |
| | | | | AAAATCCCTGGCAGAGCCTCCAGTCCAT |
| | | | | GCCCGCACCGCCTCCGCGTCCTCCCGGG |
| | | | | CTCCCCGTGGAGGGGCACCAATTTGTCCT |
| | | | | CGCCTGCGCCTGCTCGGGCCAGATGGTG |
| | | | | GGTATTTCCAGGCCACAAAGTCCTCTGA |
| | | | | CCTTTGAACAGTTGCCGCCGAATTTCAAT |
| | | | | AATGAAAGGGCCTTTTTTGAATATGTAC |
| | | | | AAATGAGACGTTATATTTCCATACATTTT |
| | | | | ATTTCCAGCCTCATCTGCGAATCTAATAT |
| | | | | TGACCC |
| chr19_ | URI1 | chr19: | chr19: | CGGCTGCTAGAGGGGAGGGAGGGGATCT |
| 30562320_ | | 30562320- | 30562250- | CGTGCCCCCGATCTGGCACCGGGGTGGG |
| 30562321 | | 30562321 | 30562681 | CAGGGCATATGGACGGCAGCCATTGGCG |
| | | | | AGGTGCCCACACCAGGCCCTGGCTTCGG |
| | | | | GCCCAGCATGAGCCTGGGCCGGCGGTGG |
| | | | | GTAAGCTCTCTATCCCTCTCTGCCCTATA |
| | | | | AAAATCCCTGGCAGAGCCTCCAGTCCAT |
| | | | | GCCCGCACCGCCTCCGCGTCCTCCCGGG |
| | | | | CTCCCCGTGGAGGGGCACCAATTTGTCCT |
| | | | | CGCCTGCGCCTGCTCGGGCCAGATGGTG |
| | | | | GGTATTTCCAGGCCACAAAGTCCTCTGA |
| | | | | CCTTTGAACAGTTGCCGCCGAATTTCAAT |
| | | | | AATGAAAGGGCCTTTTTTGAATATGTAC |
| | | | | AAATGAGACGTTATATTTCCATACATTTT |
| | | | | ATTTCCAGCCTCATCTGCGAATCTAATAT |
| | | | | TGACCC |
| chr11_ | IFITM1 | chr11: | chr11: | GAAATAGAAACTTAAGAGAAATACACAC |
| 314074_ | | 314074- | 313999- | TTCTGAGAAACTGAAACGACAGGGGAAA |
| 314075 | | 314075 | 314119 | GGAGGTCTCACTGAGCACCGTCCCAGCA |
| | | | | TCCGGACACCACAGCGGCCCTTCGCTCC |
| | | | | ACGCAGAA |
| chr6_ | HIST1H4F | chr6: | chr6: | GCCGTAACCTACACGGAGCACGCCAAGC |
| 26240975_ | | 26240975- | 26240860- | GTAAGACAGTCACTGCAATGGATGTTGT |
| 26240976 | | 26240976 | 26240980 | CTACGCGCTCAAGCGCCAGGGACGCACT |
| | | | | CTGTACGGCTTTGGTGGCTGAGCCTCACC |
| | | | | CCGGCTT |
| chr6_ | HIST1H4F | chr6: | chr6: | GCCGTAACCTACACGGAGCACGCCAAGC |
| 26240950_ | | 26240950- | 26240860- | GTAAGACAGTCACTGCAATGGATGTTGT |
| 26240951 | | 26240951 | 26240980 | CTACGCGCTCAAGCGCCAGGGACGCACT |
| | | | | CTGTACGGCTTTGGTGGCTGAGCCTCACC |
| | | | | CCGGCTT |
| chr6_ | HIST1H4F | chr6: | chr6: | GCCGTAACCTACACGGAGCACGCCAAGC |
| 26240939_ | | 26240939- | 26240860- | GTAAGACAGTCACTGCAATGGATGTTGT |
| 26240940 | | 26240940 | 26240980 | CTACGCGCTCAAGCGCCAGGGACGCACT |
| | | | | CTGTACGGCTTTGGTGGCTGAGCCTCACC |
| | | | | CCGGCTT |
| chr19_ | DIRAS1 | chr19: | chr19: | CGCCTGTAGGGTTGCTGGGAACACATGG |
| 2723189_ | | 2723189- | 2723033- | GCAGGGCGCCCACCTACACGGGGCTGCT |
| 2723190 | | 2723190 | 2723261 | GGGCACCCAAGCACTCAGACGGCAGCCC |
| | | | | TGCCCCTTCCCAGTCCGCCTGCCTGCCTT |
| | | | | CGTCTCCTGAGTGACCCACCAGCGTGGG |
| | | | | GCAGGGCGTCGGGCCGGCTCCCTGTCTC |
| | | | | CTCCGGGCACCAGCTCAGGCCGGGGCAG |
| | | | | GCTGACTCATGCGTAGGAGAGTGGGCAG |
| | | | | GGC |
| chr19_ | DIRAS1 | chr19: | chr19: | CGCCTGTAGGGTTGCTGGGAACACATGG |
| 2723184_ | | 2723184- | 2723033- | GCAGGGCGCCCACCTACACGGGGCTGCT |
| 2723185 | | 2723185 | 2723261 | GGGCACCCAAGCACTCAGACGGCAGCCC |
| | | | | TGCCCCTTCCCAGTCCGCCTGCCTGCCTT |
| | | | | CGTCTCCTGAGTGACCCACCAGCGTGGG |
| | | | | GCAGGGCGTCGGGCCGGCTCCCTGTCTC |
| | | | | CTCCGGGCACCAGCTCAGGCCGGGGCAG |
| | | | | GCTGACTCATGCGTAGGAGAGTGGGCAG |
| | | | | GGC |
| chr8_ | MIR1302-7 | chr8: | chr8: | GCTTGGCCGGATGTGGGGCTCTAGCGGG |
| 142852883_ | | 142852883- | 142852803- | GACCTCTTTCCCTGAGAACATCTGGGCCA |
| 142852884_ | | 142852884 | 142852998 | TGCGTTCGTGTCTCCCGGCACCCGGCCCT |
| CHALM | | | | GCCCTTGAGACCAACACCATCCTGATTCT |
| | | | | TGATACTTTCCGTGAAATGCGTTGGCTCA |
| | | | | CTGGTTTGGTTTTTGTCCTCTCTGGAAAC |
| | | | | TGTTCAGATGCTCTCTTTGTTC |
| chr8_ | MIR1302-7 | chr8: | chr8: | GCTTGGCCGGATGTGGGGCTCTAGCGGG |
| 142852876_ | | 142852876- | 142852803- | GACCTCTTTCCCTGAGAACATCTGGGCCA |
| 142852877_ | | 142852877 | 142852998 | TGCGTTCGTGTCTCCCGGCACCCGGCCCT |
| CHALM | | | | GCCCTTGAGACCAACACCATCCTGATTCT |
| | | | | TGATACTTTCCGTGAAATGCGTTGGCTCA |
| | | | | CTGGTTTGGTTTTTGTCCTCTCTGGAAAC |
| | | | | TGTTCAGATGCTCTCTTTGTTC |
| chr7_ | LOC100506585 | chr7: | chr7: | CTGAGTGTGGGGGCTGTGGGCCCGCTTG |
| 157563602_ | | 157563602- | 157563542- | GCCAGTTGAGGCAGCAGAATGCACCTGC |
| 157563603 | | 157563603 | 157563662 | CCCCGAGGTGAGACCTCTGTCCTCCCCAC |
| | | | | CTGAGTGCGGGGGCTGTGGGCCCGCTTG |
| | | | | GCCGGTT |
| chr11_ | IFITM1 | chr11: | chr11: | GAAATAGAAACTTAAGAGAAATACACAC |
| 314113_ | | 314113- | 313999- | TTCTGAGAAACTGAAACGACAGGGGAAA |
| 314114 | | 314114 | 314119 | GGAGGTCTCACTGAGCACCGTCCCAGCA |
| | | | | TCCGGACACCACAGCGGCCCTTCGCTCC |
| | | | | ACGCAGAA |
| chr11_ | IFITM1 | chr11: | chr11: | GAAATAGAAACTTAAGAGAAATACACAC |
| 314106_ | | 314106- | 313999- | TTCTGAGAAACTGAAACGACAGGGGAAA |
| 314107 | | 314107 | 314119 | GGAGGTCTCACTGAGCACCGTCCCAGCA |
| | | | | TCCGGACACCACAGCGGCCCTTCGCTCC |
| | | | | ACGCAGAA |
| chr11_ | IFITMI | chr11: | chr11: | GAAATAGAAACTTAAGAGAAATACACAC |
| 314098_ | | 314098- | 313999- | TTCTGAGAAACTGAAACGACAGGGGAAA |
| 314099 | | 314099 | 314119 | GGAGGTCTCACTGAGCACCGTCCCAGCA |
| | | | | TCCGGACACCACAGCGGCCCTTCGCTCC |
| | | | | ACGCAGAA |
| chr11_ | IFITMI | chr11: | chr11: | GAAATAGAAACTTAAGAGAAATACACAC |
| 314086_ | | 314086- | 313999- | TTCTGAGAAACTGAAACGACAGGGGAAA |
| 314087 | | 314087 | 314119 | GGAGGTCTCACTGAGCACCGTCCCAGCA |
| | | | | TCCGGACACCACAGCGGCCCTTCGCTCC |
| | | | | ACGCAGAA |
| chr1_ | RASSF5 | chr1: | chr1: | GGTCAGGGCTGCTGTCTTTTGAGTCAGCC |
| 206753453_ | | 206753453- | 206753393- | CTGGAAGGCAGAGGTGCCTGTGGCCACT |
| 206753454 | | 206753454 | 206753513 | TGCGTCACTTCCCCGCGTTTGTGGTGGGA |
| | | | | AGGGGAGAGATAGATGGGTCTATCTGCT |
| | | | | GAAGAA |
| chr7_ | MIR153-2 | chr7: | chr7: | TTGAGGTGACAGGCACGTGAACCGCCCT |
| 157319206_ | | 157319206- | 157319146- | GATCTGACTATCCCACATCATACACGGG |
| 157319207_ | | 157319207 | 157319266 | CGTCGAAACACCACCCTGTATTCCAGAA |
| CHALM | | | | ACAGGTGCAATTATTGTGTGCCAATTAA |
| | | | | AAAAAGGA |
| chr7_ | MIR153-2 | chr7: | chr7: | TTGAGGTGACAGGCACGTGAACCGCCCT |
| 157319203_ | | 157319203- | 157319146- | GATCTGACTATCCCACATCATACACGGG |
| 157319204_ | | 157319204 | 157319266 | CGTCGAAACACCACCCTGTATTCCAGAA |
| CHALM | | | | ACAGGTGCAATTATTGTGTGCCAATTAA |
| | | | | AAAAAGGA |
| chr7_ | MIR153-2 | chr7: | chr7: | TTGAGGTGACAGGCACGTGAACCGCCCT |
| 157319199_ | | 157319199- | 157319146- | GATCTGACTATCCCACATCATACACGGG |
| 157319200_ | | 157319200 | 157319266 | CGTCGAAACACCACCCTGTATTCCAGAA |
| CHALM | | | | ACAGGTGCAATTATTGTGTGCCAATTAA |
| | | | | AAAAAGGA |
| chr1_ | TNFAIP8L2 | chr1: | chr1: | AATGGTGGCAGAGGAATCTCTAGCTCTT |
| 151129298_ | | 151129298- | 151129238- | TCAGTCTTAGCTTTTCATATTGTGAAACT |
| 151129299 | | 151129299 | 151129358 | CTCGCTTGTACAATTCTTGGGTCCTTTGT |
| | | | | AGAGCTTAGCTTAGTTTTTCTGGTGGCAA |
| | | | | GGCAG |
| chr7_ | LAT2 | chr7: | chr7: | GGTGCTCTAAACCCTGCTTCCTGTCCCTG |
| 73641105_ | | 73641105- | 73641028- | CGCCCCACAAGGAGGCTGTGCCTAACGG |
| 73641106 | | 73641106 | 73641148 | TCTGACCCGTTTGCACGCCAGGGGCGAG |
| | | | | GGGCCGGATGCTGGGTCCTCCCCGCTTTG |
| | | | | GAGGGT |
| chr7_ | LAT2 | chr7: | chr7: | GGTGCTCTAAACCCTGCTTCCTGTCCCTG |
| 73641071_ | | 73641071- | 73641028- | CGCCCCACAAGGAGGCTGTGCCTAACGG |
| 73641072 | | 73641072 | 73641148 | TCTGACCCGTTTGCACGCCAGGGGCGAG |
| | | | | GGGCCGGATGCTGGGTCCTCCCCGCTTTG |
| | | | | GAGGGT |
| chr16_ | C16orf54 | chr16: | chr16: | CTCCTAGAGCCACTCTTCCTGGTGCTGGA |
| 29757375_ | | 29757375- | 29757258- | CTAAGAGGTGCAGGCTTGGAGGGTGCAG |
| 29757376 | | 29757376 | 29757378 | GGCGGTCCGCCTCTCAGACGTAGAGGCC |
| | | | | CGGCCTCGGATGAAGGCGGAAGGGAGG |
| | | | | GCACCGCC |
| chr16_ | C16orf54 | chr16: | chr16: | CTCCTAGAGCCACTCTTCCTGGTGCTGGA |
| 29757360_ | | 29757360- | 29757258- | CTAAGAGGTGCAGGCTTGGAGGGTGCAG |
| 29757361 | | 29757361 | 29757378 | GGCGGTCCGCCTCTCAGACGTAGAGGCC |
| | | | | CGGCCTCGGATGAAGGCGGAAGGGAGG |
| | | | | GCACCGCC |
| chr11_ | CTTN | chr11: | chr11: | GTGCACTGGGGTCACCTTAGACCACAGG |
| 70211540_ | | 70211540- | 70211458- | AAATGTCTGGTTAACACACGAAGAGATG |
| 70211541 | | 70211541 | 70211578 | GAAACGCTCGCAGCCACGCCGCAAACGG |
| | | | | TTAGTCACGCCCCACAGCCTGCACTCCTC |
| | | | | CCAGCGC |
| chr11_ | CTTN | chr11: | chr11: | GTGCACTGGGGTCACCTTAGACCACAGG |
| 70211534_ | | 70211534- | 70211458- | AAATGTCTGGTTAACACACGAAGAGATG |
| 70211535 | | 70211535 | 70211578 | GAAACGCTCGCAGCCACGCCGCAAACGG |
| | | | | TTAGTCACGCCCCACAGCCTGCACTCCTC |
| | | | | CCAGCGC |
| chr11_ | CTTN | chr11: | chr11: | GTGCACTGGGGTCACCTTAGACCACAGG |
| 70211531_ | | 70211531- | 70211458- | AAATGTCTGGTTAACACACGAAGAGATG |
| 70211532 | | 70211532 | 70211578 | GAAACGCTCGCAGCCACGCCGCAAACGG |
| | | | | TTAGTCACGCCCCACAGCCTGCACTCCTC |
| | | | | CCAGCGC |
| chr11_ | CTTN | chr11: | chr11: | GTGCACTGGGGTCACCTTAGACCACAGG |
| 70211523_ | | 70211523- | 70211458- | AAATGTCTGGTTAACACACGAAGAGATG |
| 70211524 | | 70211524 | 70211578 | GAAACGCTCGCAGCCACGCCGCAAACGG |
| | | | | TTAGTCACGCCCCACAGCCTGCACTCCTC |
| | | | | CCAGCGC |
| chr14_ | EVL | chr14: | chr14: | GTTGGGGAAGGAATGGAACCAGGGTTTA |
| 100532797_ | | 100532797- | 100532734- | ACGAGGTTGGCCTCATCCGGTGGTTTTCG |
| 100532798 | | 100532798 | 100532854 | CAGCACGCTACACAGGCACAGAAGCAGC |
| | | | | TTGCAGTGAGTTGTTAAAATTGACGTGGT |
| | | | | GATAGG |
| chr14_ | EVL | chr14: | chr14: | GTTGGGGAAGGAATGGAACCAGGGTTTA |
| 100532790_ | | 100532790- | 100532734- | ACGAGGTTGGCCTCATCCGGTGGTTTTCG |
| 100532791 | | 100532791 | 100532854 | CAGCACGCTACACAGGCACAGAAGCAGC |
| | | | | TTGCAGTGAGTTGTTAAAATTGACGTGGT |
| | | | | GATAGG |
| chr5_ | PFN3 | chr5: | chr5: | CATGCCGGGGAGGATGGAGGATCCGTGC |
| 176829777_ | | 176829777- | 176829592- | ACGTCCGGGGCTGAGCAGCGCTCCAGGG |
| 176829778 | | 176829778 | 176829808 | AGAGGAACGGTACCTGCGCCTCCTGCAG |
| | | | | GAAGCTGGCATATTCCTCCGCCCCTGCG |
| | | | | AACACAGAGCGCCTTCTTCACACCCCAT |
| | | | | CTGACAACGCTTGCCGCCCGGACGATGG |
| | | | | ACAAAGCTGCTCCAGGCGCTTGTAAACC |
| | | | | CACTCATGCCCTTCCTCTCT |
| chr5_ | PFN3 | chr5: | chr5: | CATGCCGGGGAGGATGGAGGATCCGTGC |
| 176829755_ | | 176829755- | 176829592- | ACGTCCGGGGCTGAGCAGCGCTCCAGGG |
| 176829756 | | 176829756 | 176829808 | AGAGGAACGGTACCTGCGCCTCCTGCAG |
| | | | | GAAGCTGGCATATTCCTCCGCCCCTGCG |
| | | | | AACACAGAGCGCCTTCTTCACACCCCAT |
| | | | | CTGACAACGCTTGCCGCCCGGACGATGG |
| | | | | ACAAAGCTGCTCCAGGCGCTTGTAAACC |
| | | | | CACTCATGCCCTTCCTCTCT |
| chr16_ | C16orf54 | chr16: | chr16: | CTCCTAGAGCCACTCTTCCTGGTGCTGGA |
| 29757350_ | | 29757350- | 29757258- | CTAAGAGGTGCAGGCTTGGAGGGTGCAG |
| 29757351 | | 29757351 | 29757378 | GGCGGTCCGCCTCTCAGACGTAGAGGCC |
| | | | | CGGCCTCGGATGAAGGCGGAAGGGAGG |
| | | | | GCACCGCC |
| chr16_ | C16orf54 | chr16: | chr16: | CTCCTAGAGCCACTCTTCCTGGTGCTGGA |
| 29757323_ | | 29757323- | 29757258- | CTAAGAGGTGCAGGCTTGGAGGGTGCAG |
| 29757324 | | 29757324 | 29757378 | GGCGGTCCGCCTCTCAGACGTAGAGGCC |
| | | | | CGGCCTCGGATGAAGGCGGAAGGGAGG |
| | | | | GCACCGCC |
| chr3_ | BDH1 | chr3: | chr3: | GCGAGGGCCAGGGAGATGGAGCCAGGT |
| 197283111_ | | 197283111- | 197283051- | GCGGAAAGGGTTGCAGCCTCGCGGGCTG |
| 197283112 | | 197283112 | 197283171 | AGGCCGGAGGGTGAAGGCGCCGCTCCTT |
| | | | | GAAGTGAGGAAAACTCCGGTGAGGAAG |
| | | | | AGGCTCTTTC |
| chr6_ | LOC101 | chr6: | chr6: | CTTCAGTGCACGTGGCTGTTTGTCCCCTC |
| 11976066_ | 928253 | 11976066- | 11975889- | AGAGTAGGAAGCAGCTGTTCTAACTTCC |
| 11976067 | | 11976067 | 11976084 | GTCTTCGCAGAAGGTTGTCATCTCCTCTG |
| | | | | TGGTGAGGGCTCTGCAGAGTCACTGCTT |
| | | | | GTTTGACCACATTCAGATTGCGGCCAGG |
| | | | | GCTAAAGTCACAGCTTACCTCTTCAGCTC |
| | | | | TCTACCGAACTGTATTTTCCTTGA |
| chr6_ | LOC101 | chr6: | chr6: | CTTCAGTGCACGTGGCTGTTTGTCCCCTC |
| 11976024_ | 928253 | 11976024- | 11975889- | AGAGTAGGAAGCAGCTGTTCTAACTTCC |
| 11976025 | | 11976025 | 11976084 | GTCTTCGCAGAAGGTTGTCATCTCCTCTG |
| | | | | TGGTGAGGGCTCTGCAGAGTCACTGCTT |
| | | | | GTTTGACCACATTCAGATTGCGGCCAGG |
| | | | | GCTAAAGTCACAGCTTACCTCTTCAGCTC |
| | | | | TCTACCGAACTGTATTTTCCTTGA |
| chr6_ | FOXP4- | chr6: | chr6: | CCCTGGTATTGTGCCTGTTTGGGGGAAG |
| 41528502_ | AS1 | 41528502- | 41528416- | AAAACGTCAATAAAAATTAATTGATGAG |
| 41528503 | | 41528503 | 41528536 | TTGGCAGGGCGGGCGGTGCGGGTTCGCG |
| | | | | GCGAGGCGCAGGGTGTCATGGCAAATGT |
| | | | | TACGGCTC |
| chr6_ | FOXP4- | chr6: | chr6: | CCCTGGTATTGTGCCTGTTTGGGGGAAG |
| 41528499_ | AS1 | 41528499- | 41528416- | AAAACGTCAATAAAAATTAATTGATGAG |
| 41528500 | | 41528500 | 41528536 | TTGGCAGGGCGGGCGGTGCGGGTTCGCG |
| | | | | GCGAGGCGCAGGGTGTCATGGCAAATGT |
| | | | | TACGGCTC |
| chr6_ | FOXP4- | chr6: | chr6: | CCCTGGTATTGTGCCTGTTTGGGGGAAG |
| 41528497_ | AS1 | 41528497- | 41528416- | AAAACGTCAATAAAAATTAATTGATGAG |
| 41528498 | | 41528498 | 41528536 | TTGGCAGGGCGGGCGGTGCGGGTTCGCG |
| | | | | GCGAGGCGCAGGGTGTCATGGCAAATGT |
| | | | | TACGGCTC |
| chr6_ | FOXP4- | chr6: | chr6: | CCCTGGTATTGTGCCTGTTTGGGGGAAG |
| 41528491_ | AS1 | 41528491- | 41528416- | AAAACGTCAATAAAAATTAATTGATGAG |
| 41528492 | | 41528492 | 41528536 | TTGGCAGGGCGGGCGGTGCGGGTTCGCG |
| | | | | GCGAGGCGCAGGGTGTCATGGCAAATGT |
| | | | | TACGGCTC |
| chr16_ | C16orf54 | chr16: | chr16: | CTCCTAGAGCCACTCTTCCTGGTGCTGGA |
| 29757344_ | | 29757344- | 29757258- | CTAAGAGGTGCAGGCTTGGAGGGTGCAG |
| 29757345 | | 29757345 | 29757378 | GGCGGTCCGCCTCTCAGACGTAGAGGCC |
| | | | | CGGCCTCGGATGAAGGCGGAAGGGAGG |
| | | | | GCACCGCC |
| chr16_ | C16orf54 | chr16: | chr16: | CTCCTAGAGCCACTCTTCCTGGTGCTGGA |
| 29757334_ | | 29757334- | 29757258- | CTAAGAGGTGCAGGCTTGGAGGGTGCAG |
| 29757335 | | 29757335 | 29757378 | GGCGGTCCGCCTCTCAGACGTAGAGGCC |
| | | | | CGGCCTCGGATGAAGGCGGAAGGGAGG |
| | | | | GCACCGCC |
| chr17_ | HEXDC | chr17: | chr17: | TGGTTTCCTGATGAGCAGACAAAATTCG |
| 80358932_ | | 80358932- | 80358759- | GTCAGGGGCCATGGAAGGAGCCGGGAG |
| 80358933 | | 80358933 | 80358979 | CGACCGGACCAGGGCGAATCTGCTGTCT |
| | | | | GTGCCGGCGGAAGGCCAGTAAGTGCCGT |
| | | | | GCAGTCGTGAAAAAGGAAGTGGTTAAAG |
| | | | | TGGTTACGGCAAAGCGGTG |
| | | | | GCGCCGGCTGCTCCCGACACTGCAGCCC |
| | | | | GAGAGGGAGAGAGGAGGACTGAAGGCA |
| | | | | GTCCCAG |
| chr17_ | HEXDC | chr17: | chr17: | TGGTTTCCTGATGAGCAGACAAAATTCG |
| 80358919_ | | 80358919- | 80358759- | GTCAGGGGCCATGGAAGGAGCCGGGAG |
| 80358920 | | 80358920 | 80358979 | CGACCGGACCAGGGCGAATCTGCTGTCT |
| | | | | GTGCCGGCGGAAGGCCAGTAAGTGCCGT |
| | | | | GCAGTCGTGAAAAAGGAAGTGGTTAAAG |
| | | | | TGGTTACGGCAAAGCGGTG |
| | | | | GCGCCGGCTGCTCCCGACACTGCAGCCC |
| | | | | GAGAGGGAGAGAGGAGGACTGAAGGCA |
| | | | | GTCCCAG |
| chr6_ | HEXDC | chr6: | chr6: | CCTGTGAGAGGAAGCTGCTGTGATTCAG |
| 31527920_ | | 31527920- | 31527829- | AGAAGAGACTTCAAGCTGTGTGTGACCC |
| 31527921 | | 31527921 | 31527949 | TGGCGTCCGGTTCCTCTCACAGGCTGGA |
| | | | | GCTTTTCGGAAGTGGCATGCAAAGAGTC |
| | | | | CAGGTTTG |
| chr6_ | HEXDC | chr6: | chr6: | CCTGTGAGAGGAAGCTGCTGTGATTCAG |
| 31527893_ | | 31527893- | 31527829- | AGAAGAGACTTCAAGCTGTGTGTGACCC |
| 31527894 | | 31527894 | 31527949 | TGGCGTCCGGTTCCTCTCACAGGCTGGA |
| | | | | GCTTTTCGGAAGTGGCATGCAAAGAGTC |
| | | | | CAGGTTTG |

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

A “site” corresponds to a single site, which in some cases is a single base position or a group of correlated base positions, e.g., a CpG site. A “locus” corresponds to a region that includes multiple sites. In some instances, a locus includes one site.

### EXAMPLES

These examples are provided for illustrative purposes only and not to limit the scope of the claims provided herein.

### Example 1. General Methodology

cfDNA Extraction

Genomic DNA extraction from pieces of freshly frozen healthy or cancer tissues was performed with QIAamp DNA Mini Kit (Qiagen) according to manufacturer's recommendations. DNA was extracted from roughly 0.5 mg of tissue. DNA was stored at −20° C. and analyzed within one week of preparation.

DNA Extraction from FFPE Samples

Genomic DNA from frozen FFPE samples was extracted using QIAamp DNA FFPE Tissue Kit with several modifications. DNA were stored at −20° C. for further analysis.

### Bisulfite Conversion of Genomic DNA

1 μg of genomic DNA was converted to bis-DNA using EZ DNA Methylation-Lightning™ Kit (Zymo Research) according to the manufacturer's protocol. Resulting bis-DNA had a size distribution of ˜200-3000 bp, with a peak around ˜500-1000 bp. The efficiency of bisulfite conversion was >99.8% as verified by deep-sequencing of bis-DNA and analyzing the ratio of C to T conversion of CH (non-CG) dinucleotides.

### Enzymatic Conversion of Genomic DNA

The NEB Enzymatic Methyl-seq (EM-seq) Library Preparation Kit is a new tool for identifying CpG sites without the use of damaging chemical conversion processes. Instead, EM-seq uses a two-step enzymatic conversion process that is less damaging to the DNA, resulting in high-quality libraries that can be sequenced to identify 5mC and 5hmC site.

Extracted DNA was used for library preparation using a NEBNext Ultra II Kit (NEB, Ipswich, MA USA) according to the manufacturer's instructions for DNA end repair, methylated adapter ligation, and size selection. The adapter ligated DNA fragments were deaminated by the enzymatic deamination method using Enzymatic Methyl-seq Conversion Module (NEB, E7125).

### Hybridization Target Enrichment by Using Target Specific Probes

A target enrichment protocol consisting steps for: libraries for hybridization, hybridize capture probes with pools, bind hybridized targets to streptavidin beads, post-capture PCR amplify step, purification, and QC performance step were followed. Libraries were sequenced using Illumina platforms.

### Sequencing Data Analysis

Mapping of sequencing reads was done using the software tool bisReadMapper with some modifications. First, UMI were extracted from each sequencing read and appended to read headers within FASTQ files using a custom script. Reads were on-the-fly converted as if all C were non-methylated and mapped to in-silico converted DNA strands of the human genome, also as if all C were non-methylated, using Bowtie2. Methylation frequencies were calculated for all CpG dinucleotides contained within the regions captured by padlock probes by dividing the numbers of unique reads carrying a C at the interrogated position by the total number of reads covering the interrogated position.

### Example 2. Diagnosis of Liver Cancer Utilizing a Cell-Free DNA Sample

Cell-free DNA sample was obtained from a QIAamp Circulating Nucleic Acid Kit. Methylation profile of a panel of genes and/or three-protein panel were used for the analysis.

### DNA Isolation and Quantification

Tumor and corresponding far site samples of the same tissue were obtained from patients who underwent surgical tumor resection; samples were frozen and preserved at −80° C. until use. Isolation of DNA from samples was performed using AllPrep DNA Mini kit (Qiagen, Valencia, CA) according to the manufacturer's recommendations. DNA concentration was measured using the Qubit dsDNA High Sensitivity Assay Kit (Thermo Fisher, USA) as per manufacturer's instructions.

Library Preparation and Enzymatic Conversion of cfDNA

Two reactions were performed as per manufacturer's instructions. The first reaction uses ten-eleven translocation dioxygenase 2 (TET2) and T4 phage b-glucosyltranferase (T4-bGT). TET2 is a Fe(II)/alpha-ketoglutarate-dependent dioxygenase that catalyzes the oxidization of 5-methylcytosine to 5-hydroxymethylcytosine (5hmC), 5-formylcytosine (5fC), and 5-carboxycytosine (5caC) in three consecutive steps with the concomitant formation of C02 and succinate. T4-bGT catalyzes the glucosylation of the formed 5hmC as well as pre-existing genomic 5hmCs to 5-(β-glucosyloxymethyl)cytosine (5gmC). These reactions protect 5mC and 5hmC against deamination by APOBEC3A. This ensures that only cytosines are deaminated to uracils, thus enabling the discrimination of cytosine from its methylated and hydroxymethylated forms. The following sections characterize the catalytic actions of TET2, T4-phage b glucosyltransferase (T4-bGT) and apolipoprotein B mRNA editing enzyme, catalytic polypeptide-like 3A (APOBEC3A).

The 50 μl of sheared material was transferred to a PCR strip tube to begin library construction. NEBNext DNA Ultra II Reagents (NEB, Ipswich, MA) were used according to the manufacturer's instructions for end repair, A-tailing and adaptor ligation of 0.4 μM EM-seq adaptor (A5mCA5mCT5mCTTT5mC5mC5mCTA5mCA5mCGA5mCG5mCT5mCTT5mC5mCGAT5m C*T and [Phos]GAT5mCGGAAGAG5mCA5mCA5mCGT5mCTGAA5mCT5mC5mCAGT5mCA). The ligated samples were mixed with 110 μl of resuspended NEBNext Sample Purification Beads and cleaned up according to the manufacturer's instructions. The library was eluted in 29 μl of water. DNA was oxidized in a 50 μl reaction volume containing 50 mM Tris HCl pH 8.0, 1 mM DTT, 5 mM Sodium-L-Ascorbate, 20 mM a-KG, 2 mM ATP, 50 mM Ammonium Iron (II) sulfate hexahydrate, 0.04 mM UDG (NEB, Ipswich, MA), 16 μg mTET2, 10 U T4-bGT (NEB, Ipswich, MA). The reaction was initiated by adding Fe (II) solution to a final reaction concentration of 40 μM and then incubated for 1 h at 37° C. Following this, 0.8 U of proteinase K (NEB, Ipswich, MA) was added and before incubation for 30 min at 37° C. At the end of the incubation, the DNA was purified using 90 μl of resuspended NEBNext Sample Purification Beads according to the manufacturer's instructions. DNA was eluted in 17 μl of water and 16 μl was then transferred to a new PCR tube and denatured by addition of 4 μl of formamide (Sigma-Aldrich, St. Louis, MO) and incubation at 85° C. for 10 min. The DNA was then deaminated in 50 mM Bis-Tris pH 6.0, 0.1% Triton X-100, 20 μg BSA (NEB, Ipswich, MA) using 0.2 μg of APOBEC3A. The reaction was incubated at 37° C. for 3 h and the DNA was purified using 100 μl of resuspended NEBNext Sample Purification Beads according to the manufacturer's protocol. The sample was eluted in 21 μl water and 20 μl was transferred to a new tube. 1 μM of NEBNext Unique Dual Index Primers and 25 μl NEBNext Q5U Master Mix (M0597, New England Biolabs, Ipswich, MA) were added to the DNA and amplified as follows: 98° C. for 30 s, then cycled 4 (200 ng), 6 (50 ng) and 8 (10 ng) times according to DNA input, 98° C. for 10 s, 62° C. for 30 s and 65° C. for 60 s. A final extension of 65° C. for 5 min and hold at 4° C. EM-seq libraries were purified using 45 μl of resuspended NEBNext Sample Purification Beads and the sample was eluted in 21 μl water and 20 μl was transferred to a new tube. Low input EM-seq libraries for 100 μg-10 ng gDNA inputs were processed as for the 10-200 ng gDNA inputs and used 2U T4-bGT. Libraries were quantified using D1000HS Tape for TapeStation (Agilent).

### Hybridizing the Amplified Enzymatic-Converted Library

An optimized target enrichment protocol consisting steps for: libraries for hybridization, hybridize capture probes with pools, bind hybridized targets to streptavidin beads, post-capture PCR amplify step, purification, and QC performance step were followed. Libraries were sequenced using Illumina platforms.

### Data Analysis

The methylation level, protein marker values and clinical information were used in a stepwise regression to develop a logistic regression algorithm. AFP values were logarithmized to account for extreme values. Missing values of methylation markers or AFP were imputed by random choosing from existing values. To train random forest models for HCC prediction, we randomly split the samples into training set (70%) and test set (30%). Within the training set, ten-fold cross validation was used to optimize the hyperparameters of random forest. In order to keep consistent and demonstrate each model's robustness, we used the same set of hyperparameters for all random forest models trained in this work. The R package ‘ranger’ was used for model training. Out-out-bag predictions were used for evaluating the performance of random forest models in the training set. The classifier area under the receiver operating characteristic (AUROC) curve was used for detecting and classifying patients with HCC from patients with benign liver diseases, other cancer types, and normal healthy controls. Within the validation set, the performance of models in predicting HCC was evaluated by the AUROC scores. The sensitivity at 95% specificity was calculated by using R package ‘optimal.thresholds’. The processes of dataset splitting, model training and validation were repeated for 200 times. The mean values of AUROC scores or sensitivities were reported along with 90% confidence interval calculated by bootstrapping.

### Example 3. Evaluation of the Markers in an Independent Ethnic and Geographic Cohort

An HCC-specific methylation panel was developed to build diagnostic models based on cfDNA regional methylation level by employing machine learning approaches. We evaluated the utility of DNA methylation analysis for differentiating HCC from benign liver diseases and trained the model by using a US cohort (N=136). Test performance characteristics were then evaluated in an independent ethnic and geographic cohort (N=253) in China, including 101 HCC patients, 152 Non-HCC, and a control group with 79 individuals with benign liver diseases. Area under the receiver operating characteristic curve (AUC-ROC) was used to evaluate diagnostic performance.

A random forest modeling analysis was performed to generate predictive probability of disease in the US training cohort. The HCC-specific panel of methylation biomarkers showed an AUC of 0.800 for detecting HCC from benign liver diseases. The panel had similar performance in Chinese validation cohort, which showed a consistent AUC of 0.917 with an overall sensitivity of 80.2% at 90% specificity for detecting HCC from benign liver diseases (PPV=0.910). The model performed equally well in detecting early-stage HCC, and it yielded a sensitivity of 70.8% for the stage I HCC at 90% specificity.

Table 7 provides AUC information for specific DIRs in the comparison between HCC versus non-HCC by stages, wherein different methods were compared.

Table 8 provides AUC information for specific DIRs in the comparison between HCC versus benign by stages, wherein different methods were compared.

Table 9 provides sensitivities at 90% specificity for the multi-analyte HCC test for hepatocellular carcinoma (HCC) compared to other biomarker-based tests and by stages (comparison between HCC versus non-HCC).

Table 10 provides sensitivities at 90% specificity for the multi-analyte HCC test for hepatocellular carcinoma (HCC) compared to other biomarker-based tests and by stages (comparison between HCC versus benign).

Further, multiple alternative marker combination were modeled to optimize the prediction score by using additional independent cohorts.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Tables

TABLE 1
The table illustrates the respective gene names referenced by the CpG sites described
herein.

| Gene | Note | Ref. |
|---|---|---|
| UBE4B | The modification of proteins with ubiquitin is an important cellular | RefSeq, |
| | mechanism for targeting abnormal or short-lived proteins for | Jul 2008 |
| | degradation. This gene is also the strongest candidate in the | |
| | neuroblastoma tumor suppressor genes. Alternatively spliced | |
| | transcript variants encoding distinct isoforms have been found for | |
| | this gene. | |
| TNFAIP8L2 | Acts as a negative regulator of innate and adaptive immunity by | |
| (SCNM1) | maintaining immune homeostasis. Inhibits JUN/AP1 and NF-kappa- | |
| | B activation. | |
| ADCY10 | The protein encoded by this gene belongs to a distinct class of | RefSeq, |
| | adenylyl cyclases that is soluble and insensitive to G protein or | Jul 2014 |
| | forskolin regulation. Variation at this gene has been observed in | |
| | patients with absorptive hypercalciuria. | |
| RASSF5 | This gene is a member of the Ras association domain family. It | RefSeq, |
| | functions as a tumor suppressor, and is inactivated in a variety of | Jul 2008 |
| | cancers. | |
| RPS6KA1 | This gene encodes a member of the RSK (ribosomal S6 kinase) | RefSeq, |
| | family of serine/threonine kinases. This kinase contains 2 | Jul 2008 |
| | nonidentical kinase catalytic domains and phosphorylates various | |
| | substrates, including members of the mitogen-activated kinase | |
| | (MAPK) signalling pathway. | |
| FXYD6 | This gene encodes a member of the FXYD family of | RefSeq, |
| | transmembrane proteins. This particular protein encodes | Feb 2011 |
| | phosphohippolin, which likely affects the activity of Na,K-ATPase. | |
| | Multiple alternatively spliced transcript variants encoding the same | |
| | protein have been described. | |
| IFITM1 | IFN-induced antiviral protein which inhibits the entry of viruses to | |
| | the host cell cytoplasm, permitting endocytosis, but preventing | |
| | subsequent viral fusion and release of viral contents into the cytosol. | |
| | Plays a key role in the antiproliferative action of IFN-gamma either | |
| | by inhibiting the ERK activation or by arresting cell growth in G1 | |
| | phase in a p53-dependent manner. Acts as a positive regulator of | |
| | osteoblast differentiation. | |
| PPFIA1 | The protein encoded by this gene is a member of the LAR protein- | RefSeq, |
| | tyrosine phosphatase-interacting protein (liprin) family. This | Jul 2008 |
| | interaction may regulate the disassembly of focal adhesion and thus | |
| | help orchestrate cell-matrix interactions. Alternatively spliced | |
| | transcript variants encoding distinct isoforms have been described. | |
| BOLA2 | This gene is located within a region of a segmental duplication on | RefSeq, |
| | chromosome 16 and is identical to BOLA2B (bolA family member | Feb 2016 |
| | 2B. Transcripts initiating at this locus may extend into downstream | |
| | SMG1 pseudogene 6 (SMG1P6) and encode fusion proteins with a | |
| | C-terminus related to SMG1 phosphatidylinositol 3-kinase-related | |
| | kinase. | |
| CHAD | Chondroadherin is a cartilage matrix protein thought to mediate | RefSeq, |
| | adhesion of isolated chondrocytes. The protein contains 11 leucine- | Jul 2008 |
| | rich repeats flanked by cysteine-rich regions. The chondroadherin | |
| | messenger RNA is present in chondrocytes at all ages. | |
| VMP1 | This gene encodes a transmembrane protein that plays a key | RefSeq, |
| | regulatory role in the process of autophagy. This gene is | Jul 2016 |
| | overexpressed in pancreatitis affected acinar cells where the | |
| | encoded protein mediates sequestration and degradation of | |
| | potentially deleterious activated zymogen granules in a process | |
| | termed, zymophagy. | |
| OGFOD3 | 2-oxoglutarate and iron dependent oxygenase domain containing 3 | |
| PSD4 | Guanine nucleotide exchange factor for ARF6 and ARL14/ARF7. | |
| | Through ARL14 activation, controls the movement of MHC | |
| | class II-containing vesicles along the actin cytoskeleton in dendritic | |
| | cells. Involved in membrane recycling. Interacts with several | |
| | phosphatidylinositol phosphate species. | |
| RUNX1 | Core binding factor (CBF) is a heterodimeric transcription factor | RefSeq, |
| | that binds to the core element of many enhancers and promoters. | Jul 2008 |
| | The protein encoded by this gene represents the alpha subunit of | |
| | CBF and is thought to be involved in the development of normal | |
| | hematopoiesis. Chromosomal translocations involving this gene are | |
| | well-documented and have been associated with several types of | |
| | leukemia | |
| B4GALT4 | This gene is one of seven beta-1,4-galactosyltransferase | RefSeq, |
| | (beta4GalT) genes. Each beta4GalT has a distinct function in the | Jul 2008 |
| | biosynthesis of different glycoconjugates and saccharide structures. | |
| | The enzyme encoded by this gene appears to mainly play a role in | |
| | glycolipid biosynthesis. Two alternatively spliced transcript variants | |
| | have been found for this gene. | |
| H3K27ac | H3K27ac is an epigenetic modification to the DNA packaging | |
| | protein Histone H3. H3K27ac is associated with the higher | |
| | activation of transcription and therefore defined as | |
| | an active enhancer mark. H3K27ac is found at both proximal and | |
| | distal regions of transcription start site (TSS). | |
| F12 | This gene encodes coagulation factor XII which circulates in blood | RefSeq, |
| | as a zymogen. Defects in this gene do not cause any clinical | Feb 2008 |
| | symptoms and the sole effect is that whole-blood clotting time is | |
| | prolonged. F12 is prognostic, high expression is favorable in liver | |
| | cancer | |
| ACTR1 | This gene encodes a 42.6 kD subunit of dynactin, a macromolecular | RefSeq, |
| | complex consisting of 10-11 subunits ranging in size from 22 to 150 | Jul 2008 |
| | kD. It is involved in a diverse array of cellular functions, including | |
| | ER-to-Golgi transport, the centripetal movement of lysosomes and | |
| | endosomes, spindle formation, chromosome movement, nuclear | |
| | positioning, and axonogenesis. | |
| PRRT1 | Proline rich transmembrane protein 1 | |
| FOXP4 | This gene belongs to subfamily P of the forkhead box (FOX) | RefSeq, |
| (AS1) | transcription factor family. Forkhead box transcription factors play | Jul 2008 |
| | important roles in the regulation of tissue- and cell type-specific | |
| | gene transcription during both development and adulthood. This | |
| | gene may play a role in the development of tumors of the kidney | |
| | and larynx. Alternative splicing of this gene produces multiple | |
| | transcript variants, some encoding different isoforms. | |
| SND1 | This gene encodes a transcriptional co-activator that interacts with | RefSeq, |
| | the acidic domain of Epstein-Barr virus nuclear antigen 2 (EBNA | Jul 2016 |
| | 2), a transcriptional activator that is required for B-lymphocyte | |
| | transformation. This protein is also thought to be essential for | |
| | normal cell growth. A similar protein in mammals and other | |
| | organisms is a component of the RNA-induced silencing complex | |
| | (RISC). | |
| PTPRN2 | This gene encodes a protein with sequence similarity to receptor- | RefSeq, |
| | like protein tyrosine phosphatases. This protein has been identified | Feb 2015 |
| | as an autoantigen in insulin-dependent diabetes mellitus. Alternative | |
| | splicing results in multiple transcript variants. | |
| LAT2 | This gene is one of the contiguous genes at 7q11.23 commonly | RefSeq, |
| | deleted in Williams syndrome, a multisystem developmental | Jul 2008 |
| | disorder. This gene consists of at least 14 exons, and its alternative | |
| | splicing generates 3 transcript variants, all encoding the same | |
| | protein. | |

TABLE 2
The table shows the makers selected based on AUC and meth_diff
cutoff (both traditional method and CHALM).

| Chr# | Start | Stop | Length | Position |
|---|---|---|---|---|
| chr1 | 2827773 | 2827893 | 120 | chr1:2827773-2827893 |
| chr1 | 9779249 | 9779369 | 120 | chr1:9779249-9779369 |
| chr1 | 13881401 | 13881521 | 120 | chr1:13881401-13881521 |
| chr1 | 20669845 | 20669965 | 120 | chr1:20669845-20669965 |
| chr1 | 26872458 | 26872578 | 120 | chr1:26872458-26872578 |
| chr1 | 27189210 | 27189330 | 120 | chr1:27189210-27189330 |
| chr1 | 27189604 | 27189724 | 120 | chr1:27189604-27189724 |
| chr1 | 42385881 | 42386001 | 120 | chr1:42385881-42386001 |
| chr1 | 43250631 | 43250751 | 120 | chr1:43250631-43250751 |
| chr1 | 43390645 | 43390765 | 120 | chr1:43390645-43390765 |
| chr1 | 47910783 | 47910903 | 120 | chr1:47910783-47910903 |
| chr1 | 92150444 | 92150564 | 120 | chr1:92150444-92150564 |
| chr1 | 102694577 | 102694697 | 120 | chr1:102694577-102694697 |
| chr1 | 111217437 | 111217557 | 120 | chr1:111217437-111217557 |
| chr1 | 119532020 | 119532140 | 120 | chr1:119532020-119532140 |
| chr1 | 119532132 | 119532252 | 120 | chr1:119532132-119532252 |
| chr1 | 119532276 | 119532396 | 120 | chr1:119532276-119532396 |
| chr1 | 119535868 | 119535988 | 120 | chr1:119535868-119535988 |
| chr1 | 145395693 | 145395813 | 120 | chr1:145395693-145395813 |
| chr1 | 149400232 | 149400352 | 120 | chr1:149400232-149400352 |
| chr1 | 151129238 | 151129358 | 120 | chr1:151129238-151129358 |
| chr1 | 151660512 | 151660632 | 120 | chr1:151660512-151660632 |
| chr1 | 151660727 | 151660847 | 120 | chr1:151660727-151660847 |
| chr1 | 152553184 | 152553304 | 120 | chr1:152553184-152553304 |
| chr1 | 152798852 | 152798972 | 120 | chr1:152798852-152798972 |
| chr1 | 152881755 | 152881875 | 120 | chr1:152881755-152881875 |
| chr1 | 152881870 | 152881990 | 120 | chr1:152881870-152881990 |
| chr1 | 152882360 | 152882480 | 120 | chr1:152882360-152882480 |
| chr1 | 152941943 | 152942063 | 120 | chr1:152941943-152942063 |
| chr1 | 152956126 | 152956246 | 120 | chr1:152956126-152956246 |
| chr1 | 153029879 | 153029999 | 120 | chr1:153029879-153029999 |
| chr1 | 153176972 | 153177092 | 120 | chr1:153176972-153177092 |
| chr1 | 153363429 | 153363549 | 120 | chr1:153363429-153363549 |
| chr1 | 153521849 | 153521969 | 120 | chr1:153521849-153521969 |
| chr1 | 153926655 | 153926775 | 120 | chr1:153926655-153926775 |
| chr1 | 154475113 | 154475233 | 120 | chr1:154475113-154475233 |
| chr1 | 154769054 | 154769174 | 120 | chr1:154769054-154769174 |
| chr1 | 154769166 | 154769286 | 120 | chr1:154769166-154769286 |
| chr1 | 155146380 | 155146500 | 120 | chr1:155146380-155146500 |
| chr1 | 156877829 | 156877949 | 120 | chr1:156877829-156877949 |
| chr1 | 157669051 | 157669171 | 120 | chr1:157669051-157669171 |
| chr1 | 157670650 | 157670770 | 120 | chr1:157670650-157670770 |
| chr1 | 157738398 | 157738518 | 120 | chr1:157738398-157738518 |
| chr1 | 158259866 | 158259986 | 120 | chr1:158259866-158259986 |
| chr1 | 158390589 | 158390709 | 120 | chr1:158390589-158390709 |
| chr1 | 160545088 | 160545208 | 120 | chr1:160545088-160545208 |
| chr1 | 161275343 | 161275463 | 120 | chr1:161275343-161275463 |
| chr1 | 161275464 | 161275584 | 120 | chr1:161275464-161275584 |
| chr1 | 161275573 | 161275693 | 120 | chr1:161275573-161275693 |
| chr1 | 161275683 | 161275803 | 120 | chr1:161275683-161275803 |
| chr1 | 161275780 | 161275900 | 120 | chr1:161275780-161275900 |
| chr1 | 161275901 | 161276021 | 120 | chr1:161275901-161276021 |
| chr1 | 161276010 | 161276130 | 120 | chr1:161276010-161276130 |
| chr1 | 161276120 | 161276240 | 120 | chr1:161276120-161276240 |
| chr1 | 161451891 | 161452011 | 120 | chr1:161451891-161452011 |
| chr1 | 161451965 | 161452085 | 120 | chr1:161451965-161452085 |
| chr1 | 163392990 | 163393110 | 120 | chr1:163392990-163393110 |
| chr1 | 166916949 | 166917069 | 120 | chr1:166916949-166917069 |
| chr1 | 167882380 | 167882500 | 120 | chr1:167882380-167882500 |
| chr1 | 167883238 | 167883358 | 120 | chr1:167883238-167883358 |
| chr1 | 169555962 | 169556082 | 120 | chr1:169555962-169556082 |
| chr1 | 171810912 | 171811032 | 120 | chr1:171810912-171811032 |
| chr1 | 177034124 | 177034244 | 120 | chr1:177034124-177034244 |
| chr1 | 177057571 | 177057691 | 120 | chr1:177057571-177057691 |
| chr1 | 177144233 | 177144353 | 120 | chr1:177144233-177144353 |
| chr1 | 177724596 | 177724716 | 120 | chr1:177724596-177724716 |
| chr1 | 180123162 | 180123282 | 120 | chr1:180123162-180123282 |
| chr1 | 181767676 | 181767796 | 120 | chr1:181767676-181767796 |
| chr1 | 184197072 | 184197192 | 120 | chr1:184197072-184197192 |
| chr1 | 201857531 | 201857651 | 120 | chr1:201857531-201857651 |
| chr1 | 203275266 | 203275386 | 120 | chr1:203275266-203275386 |
| chr1 | 206753393 | 206753513 | 120 | chr1:206753393-206753513 |
| chr1 | 209105828 | 209105948 | 120 | chr1:209105828-209105948 |
| chr1 | 211526633 | 211526753 | 120 | chr1:211526633-211526753 |
| chr1 | 213090124 | 213090244 | 120 | chr1:213090124-213090244 |
| chr1 | 220697555 | 220697675 | 120 | chr1:220697555-220697675 |
| chr1 | 230285939 | 230286059 | 120 | chr1:230285939-230286059 |
| chr1 | 235116671 | 235116791 | 120 | chr1:235116671-235116791 |
| chr1 | 238051391 | 238051511 | 120 | chr1:238051391-238051511 |
| chr1 | 238053768 | 238053888 | 120 | chr1:238053768-238053888 |
| chr1 | 239376333 | 239376453 | 120 | chr1:239376333-239376453 |
| chr1 | 239376457 | 239376577 | 120 | chr1:239376457-239376577 |
| chr1 | 240874886 | 240875006 | 120 | chr1:240874886-240875006 |
| chr1 | 240989248 | 240989368 | 120 | chr1:240989248-240989368 |
| chr1 | 242107957 | 242108077 | 120 | chr1:242107957-242108077 |
| chr1 | 242108079 | 242108199 | 120 | chr1:242108079-242108199 |
| chr1 | 247805195 | 247805315 | 120 | chr1:247805195-247805315 |
| chr1 | 247876028 | 247876148 | 120 | chr1:247876028-247876148 |
| chr1 | 247921508 | 247921628 | 120 | chr1:247921508-247921628 |
| chr1 | 248102782 | 248102902 | 120 | chr1:248102782-248102902 |
| chr1 | 248366339 | 248366459 | 120 | chr1:248366339-248366459 |
| chr1 | 248651781 | 248651901 | 120 | chr1:248651781-248651901 |
| chr10 | 1981376 | 1981496 | 120 | chr10:1981376-1981496 |
| chr10 | 2357527 | 2357647 | 120 | chr10:2357527-2357647 |
| chr10 | 2699297 | 2699417 | 120 | chr10:2699297-2699417 |
| chr10 | 8095765 | 8095885 | 120 | chr10:8095765-8095885 |
| chr10 | 8203831 | 8203951 | 120 | chr10:8203831-8203951 |
| chr10 | 8203981 | 8204101 | 120 | chr10:8203981-8204101 |
| chr10 | 15528348 | 15528468 | 120 | chr10:15528348-15528468 |
| chr10 | 30818504 | 30818624 | 120 | chr10:30818504-30818624 |
| chr10 | 70321899 | 70322019 | 120 | chr10:70321899-70322019 |
| chr10 | 71891063 | 71891183 | 120 | chr10:71891063-71891183 |
| chr10 | 101605964 | 101606084 | 120 | chr10:101605964-101606084 |
| chr10 | 102792189 | 102792309 | 120 | chr10:102792189-102792309 |
| chr10 | 102883045 | 102883165 | 120 | chr10:102883045-102883165 |
| chr10 | 102894983 | 102895103 | 120 | chr10:102894983-102895103 |
| chr10 | 103911532 | 103911652 | 120 | chr10:103911532-103911652 |
| chr10 | 111213903 | 111214023 | 120 | chr10:111213903-111214023 |
| chr10 | 122351577 | 122351697 | 120 | chr10:122351577-122351697 |
| chr10 | 126826942 | 126827062 | 120 | chr10:126826942-126827062 |
| chr10 | 127887874 | 127887994 | 120 | chr10:127887874-127887994 |
| chr10 | 127887961 | 127888081 | 120 | chr10:127887961-127888081 |
| chr10 | 130185282 | 130185402 | 120 | chr10:130185282-130185402 |
| chr10 | 130844149 | 130844269 | 120 | chr10:130844149-130844269 |
| chr10 | 132284846 | 132284966 | 120 | chr10:132284846-132284966 |
| chr10 | 133871384 | 133871504 | 120 | chr10:133871384-133871504 |
| chr10 | 133909889 | 133910009 | 120 | chr10:133909889-133910009 |
| chr10 | 134598292 | 134598412 | 120 | chr10:134598292-134598412 |
| chr10 | 134599781 | 134599901 | 120 | chr10:134599781-134599901 |
| chr10 | 134600029 | 134600149 | 120 | chr10:134600029-134600149 |
| chr11 | 289714 | 289834 | 120 | chr11:289714-289834 |
| chr11 | 289867 | 289987 | 120 | chr11:289867-289987 |
| chr11 | 313999 | 314119 | 120 | chr11:313999-314119 |
| chr11 | 314281 | 314401 | 120 | chr11:314281-314401 |
| chr11 | 314433 | 314553 | 120 | chr11:314433-314553 |
| chr11 | 1254144 | 1254264 | 120 | chr11:1254144-1254264 |
| chr11 | 1892247 | 1892367 | 120 | chr11:1892247-1892367 |
| chr11 | 2498330 | 2498450 | 120 | chr11:2498330-2498450 |
| chr11 | 5019789 | 5019909 | 120 | chr11:5019789-5019909 |
| chr11 | 5290686 | 5290806 | 120 | chr11:5290686-5290806 |
| chr11 | 5322916 | 5323036 | 120 | chr11:5322916-5323036 |
| chr11 | 6462050 | 6462170 | 120 | chr11:6462050-6462170 |
| chr11 | 7326930 | 7327050 | 120 | chr11:7326930-7327050 |
| chr11 | 21043180 | 21043300 | 120 | chr11:21043180-21043300 |
| chr11 | 59634216 | 59634336 | 120 | chr11:59634216-59634336 |
| chr11 | 61536940 | 61537060 | 120 | chr11:61536940-61537060 |
| chr11 | 65849069 | 65849189 | 120 | chr11:65849069-65849189 |
| chr11 | 66494117 | 66494237 | 120 | chr11:66494117-66494237 |
| chr11 | 67351212 | 67351332 | 120 | chr11:67351212-67351332 |
| chr11 | 69420668 | 69420788 | 120 | chr11:69420668-69420788 |
| chr11 | 70211458 | 70211578 | 120 | chr11:70211458-70211578 |
| chr11 | 70268668 | 70268788 | 120 | chr11:70268668-70268788 |
| chr11 | 94822538 | 94822658 | 120 | chr11:94822538-94822658 |
| chr11 | 108918225 | 108918345 | 120 | chr11:108918225-108918345 |
| chr11 | 113033481 | 113033601 | 120 | chr11:113033481-113033601 |
| chr11 | 116147520 | 116147640 | 120 | chr11:116147520-116147640 |
| chr11 | 116662740 | 116662860 | 120 | chr11:116662740-116662860 |
| chr11 | 127811544 | 127811664 | 120 | chr11:127811544-127811664 |
| chr11 | 129444441 | 129444561 | 120 | chr11:129444441-129444561 |
| chr11 | 132177593 | 132177713 | 120 | chr11:132177593-132177713 |
| chr12 | 2273059 | 2273179 | 120 | chr12:2273059-2273179 |
| chr12 | 5211801 | 5211921 | 120 | chr12:5211801-5211921 |
| chr12 | 7276300 | 7276420 | 120 | chr12:7276300-7276420 |
| chr12 | 10223744 | 10223864 | 120 | chr12:10223744-10223864 |
| chr12 | 10782259 | 10782379 | 120 | chr12:10782259-10782379 |
| chr12 | 21810398 | 21810518 | 120 | chr12:21810398-21810518 |
| chr12 | 25611115 | 25611235 | 120 | chr12:25611115-25611235 |
| chr12 | 32333375 | 32333495 | 120 | chr12:32333375-32333495 |
| chr12 | 52401154 | 52401274 | 120 | chr12:52401154-52401274 |
| chr12 | 53142482 | 53142602 | 120 | chr12:53142482-53142602 |
| chr12 | 93966918 | 93967038 | 120 | chr12:93966918-93967038 |
| chr12 | 95941928 | 95942048 | 120 | chr12:95941928-95942048 |
| chr12 | 95942847 | 95942967 | 120 | chr12:95942847-95942967 |
| chr12 | 121893709 | 121893829 | 120 | chr12:121893709-121893829 |
| chr12 | 122277300 | 122277420 | 120 | chr12:122277300-122277420 |
| chr12 | 123259729 | 123259849 | 120 | chr12:123259729-123259849 |
| chr12 | 126142836 | 126142956 | 120 | chr12:126142836-126142956 |
| chr12 | 128366080 | 128366200 | 120 | chr12:128366080-128366200 |
| chr12 | 129754247 | 129754367 | 120 | chr12:129754247-129754367 |
| chr12 | 130469403 | 130469523 | 120 | chr12:130469403-130469523 |
| chr12 | 130711651 | 130711771 | 120 | chr12:130711651-130711771 |
| chr12 | 131759425 | 131759545 | 120 | chr12:131759425-131759545 |
| chr12 | 131759552 | 131759672 | 120 | chr12:131759552-131759672 |
| chr12 | 132102128 | 132102248 | 120 | chr12:132102128-132102248 |
| chr12 | 132652413 | 132652533 | 120 | chr12:132652413-132652533 |
| chr12 | 132908679 | 132908799 | 120 | chr12:132908679-132908799 |
| chr12 | 132928865 | 132928985 | 120 | chr12:132928865-132928985 |
| chr12 | 133485266 | 133485386 | 120 | chr12:133485266-133485386 |
| chr12 | 133485443 | 133485563 | 120 | chr12:133485443-133485563 |
| chr12 | 133485600 | 133485720 | 120 | chr12:133485600-133485720 |
| chr13 | 24788136 | 24788256 | 120 | chr13:24788136-24788256 |
| chr13 | 39264905 | 39265025 | 120 | chr13:39264905-39265025 |
| chr13 | 39265158 | 39265278 | 120 | chr13:39265158-39265278 |
| chr13 | 53508032 | 53508152 | 120 | chr13:53508032-53508152 |
| chr13 | 96204918 | 96205038 | 120 | chr13:96204918-96205038 |
| chr13 | 97793992 | 97794112 | 120 | chr13:97793992-97794112 |
| chr13 | 108237905 | 108238025 | 120 | chr13:108237905-108238025 |
| chr13 | 112980616 | 112980736 | 120 | chr13:112980616-112980736 |
| chr13 | 113097573 | 113097693 | 120 | chr13:113097573-113097693 |
| chr14 | 54423373 | 54423493 | 120 | chr14:54423373-54423493 |
| chr14 | 55647411 | 55647531 | 120 | chr14:55647411-55647531 |
| chr14 | 69472592 | 69472712 | 120 | chr14:69472592-69472712 |
| chr14 | 72945401 | 72945521 | 120 | chr14:72945401-72945521 |
| chr14 | 93406022 | 93406142 | 120 | chr14:93406022-93406142 |
| chr14 | 100789564 | 100789684 | 120 | chr14:100789564-100789684 |
| chr14 | 103467738 | 103467858 | 120 | chr14:103467738-103467858 |
| chr14 | 104862499 | 104862619 | 120 | chr14:104862499-104862619 |
| chr14 | 106410627 | 106410747 | 120 | chr14:106410627-106410747 |
| chr14 | 106411109 | 106411229 | 120 | chr14:106411109-106411229 |
| chr14 | 106411229 | 106411349 | 120 | chr14:106411229-106411349 |
| chr14 | 106411349 | 106411469 | 120 | chr14:106411349-106411469 |
| chr14 | 106411470 | 106411590 | 120 | chr14:106411470-106411590 |
| chr14 | 106411591 | 106411711 | 120 | chr14:106411591-106411711 |
| chr14 | 106411711 | 106411831 | 120 | chr14:106411711-106411831 |
| chr14 | 106411831 | 106411951 | 120 | chr14:106411831-106411951 |
| chr14 | 106411952 | 106412072 | 120 | chr14:106411952-106412072 |
| chr14 | 106412073 | 106412193 | 120 | chr14:106412073-106412193 |
| chr14 | 106412313 | 106412433 | 120 | chr14:106412313-106412433 |
| chr14 | 106412434 | 106412554 | 120 | chr14:106412434-106412554 |
| chr14 | 106412555 | 106412675 | 120 | chr14:106412555-106412675 |
| chr14 | 106412675 | 106412795 | 120 | chr14:106412675-106412795 |
| chr14 | 106892243 | 106892363 | 120 | chr14:106892243-106892363 |
| chr15 | 22798926 | 22799046 | 120 | chr15:22798926-22799046 |
| chr15 | 31528935 | 31529055 | 120 | chr15:31528935-31529055 |
| chr15 | 32933944 | 32934064 | 120 | chr15:32933944-32934064 |
| chr15 | 35043644 | 35043764 | 120 | chr15:35043644-35043764 |
| chr15 | 40566820 | 40566940 | 120 | chr15:40566820-40566940 |
| chr15 | 45422002 | 45422122 | 120 | chr15:45422002-45422122 |
| chr15 | 65341901 | 65342021 | 120 | chr15:65341901-65342021 |
| chr15 | 66999649 | 66999769 | 120 | chr15:66999649-66999769 |
| chr15 | 70053338 | 70053458 | 120 | chr15:70053338-70053458 |
| chr15 | 73921325 | 73921445 | 120 | chr15:73921325-73921445 |
| chr15 | 76627606 | 76627726 | 120 | chr15:76627606-76627726 |
| chr15 | 83776374 | 83776494 | 120 | chr15:83776374-83776494 |
| chr15 | 86842570 | 86842690 | 120 | chr15:86842570-86842690 |
| chr15 | 91129397 | 91129517 | 120 | chr15:91129397-91129517 |
| chr15 | 93973861 | 93973981 | 120 | chr15:93973861-93973981 |
| chr15 | 94426948 | 94427068 | 120 | chr15:94426948-94427068 |
| chr15 | 99193961 | 99194081 | 120 | chr15:99193961-99194081 |
| chr15 | 102193356 | 102193476 | 120 | chr15:102193356-102193476 |
| chr16 | 230281 | 230401 | 120 | chr16:230281-230401 |
| chr16 | 1133014 | 1133134 | 120 | chr16:1133014-1133134 |
| chr16 | 4738508 | 4738628 | 120 | chr16:4738508-4738628 |
| chr16 | 7382460 | 7382580 | 120 | chr16:7382460-7382580 |
| chr16 | 9107139 | 9107259 | 120 | chr16:9107139-9107259 |
| chr16 | 15596309 | 15596429 | 120 | chr16:15596309-15596429 |
| chr16 | 27437832 | 27437952 | 120 | chr16:27437832-27437952 |
| chr16 | 29757258 | 29757378 | 120 | chr16:29757258-29757378 |
| chr16 | 56659723 | 56659843 | 120 | chr16:56659723-56659843 |
| chr16 | 56660061 | 56660181 | 120 | chr16:56660061-56660181 |
| chr16 | 56660391 | 56660511 | 120 | chr16:56660391-56660511 |
| chr16 | 56660738 | 56660858 | 120 | chr16:56660738-56660858 |
| chr16 | 69760868 | 69760988 | 120 | chr16:69760868-69760988 |
| chr16 | 86715092 | 86715212 | 120 | chr16:86715092-86715212 |
| chr16 | 86741360 | 86741480 | 120 | chr16:86741360-86741480 |
| chr16 | 87682082 | 87682202 | 120 | chr16:87682082-87682202 |
| chr16 | 90092695 | 90092815 | 120 | chr16:90092695-90092815 |
| chr17 | 2863722 | 2863842 | 120 | chr17:2863722-2863842 |
| chr17 | 3135298 | 3135418 | 120 | chr17:3135298-3135418 |
| chr17 | 6899237 | 6899357 | 120 | chr17:6899237-6899357 |
| chr17 | 9808062 | 9808182 | 120 | chr17:9808062-9808182 |
| chr17 | 29297681 | 29297801 | 120 | chr17:29297681-29297801 |
| chr17 | 29298124 | 29298244 | 120 | chr17:29298124-29298244 |
| chr17 | 31437589 | 31437709 | 120 | chr17:31437589-31437709 |
| chr17 | 31594409 | 31594529 | 120 | chr17:31594409-31594529 |
| chr17 | 38470840 | 38470960 | 120 | chr17:38470840-38470960 |
| chr17 | 39624028 | 39624148 | 120 | chr17:39624028-39624148 |
| chr17 | 41221514 | 41221634 | 120 | chr17:41221514-41221634 |
| chr17 | 48545890 | 48546010 | 120 | chr17:48545890-48546010 |
| chr17 | 48796455 | 48796575 | 120 | chr17:48796455-48796575 |
| chr17 | 54912083 | 54912203 | 120 | chr17:54912083-54912203 |
| chr17 | 55456475 | 55456595 | 120 | chr17:55456475-55456595 |
| chr17 | 57915657 | 57915777 | 120 | chr17:57915657-57915777 |
| chr17 | 59444313 | 59444433 | 120 | chr17:59444313-59444433 |
| chr17 | 59444386 | 59444506 | 120 | chr17:59444386-59444506 |
| chr17 | 59444507 | 59444627 | 120 | chr17:59444507-59444627 |
| chr17 | 59444604 | 59444724 | 120 | chr17:59444604-59444724 |
| chr17 | 65990757 | 65990877 | 120 | chr17:65990757-65990877 |
| chr17 | 71431713 | 71431833 | 120 | chr17:71431713-71431833 |
| chr17 | 73679923 | 73680043 | 120 | chr17:73679923-73680043 |
| chr17 | 76858188 | 76858308 | 120 | chr17:76858188-76858308 |
| chr17 | 76921777 | 76921897 | 120 | chr17:76921777-76921897 |
| chr17 | 77115573 | 77115693 | 120 | chr17:77115573-77115693 |
| chr17 | 77174060 | 77174180 | 120 | chr17:77174060-77174180 |
| chr17 | 78441896 | 78442016 | 120 | chr17:78441896-78442016 |
| chr17 | 78912705 | 78912825 | 120 | chr17:78912705-78912825 |
| chr17 | 80260266 | 80260386 | 120 | chr17:80260266-80260386 |
| chr17 | 80329904 | 80330024 | 120 | chr17:80329904-80330024 |
| chr17 | 80358759 | 80358879 | 120 | chr17:80358759-80358879 |
| chr17 | 80358859 | 80358979 | 120 | chr17:80358859-80358979 |
| chr18 | 3845625 | 3845745 | 120 | chr18:3845625-3845745 |
| chr18 | 3881487 | 3881607 | 120 | chr18:3881487-3881607 |
| chr18 | 5145326 | 5145446 | 120 | chr18:5145326-5145446 |
| chr18 | 13382080 | 13382200 | 120 | chr18:13382080-13382200 |
| chr18 | 32847506 | 32847626 | 120 | chr18:32847506-32847626 |
| chr18 | 43405433 | 43405553 | 120 | chr18:43405433-43405553 |
| chr18 | 44259913 | 44260033 | 120 | chr18:44259913-44260033 |
| chr18 | 55019783 | 55019903 | 120 | chr18:55019783-55019903 |
| chr18 | 73140981 | 73141101 | 120 | chr18:73140981-73141101 |
| chr18 | 75691237 | 75691357 | 120 | chr18:75691237-75691357 |
| chr18 | 77201912 | 77202032 | 120 | chr18:77201912-77202032 |
| chr19 | 1102615 | 1102735 | 120 | chr19:1102615-1102735 |
| chr19 | 12305802 | 12305922 | 120 | chr19:12305802-12305922 |
| chr19 | 12978646 | 12978766 | 120 | chr19:12978646-12978766 |
| chr19 | 14224326 | 14224446 | 120 | chr19:14224326-14224446 |
| chr19 | 14224444 | 14224564 | 120 | chr19:14224444-14224564 |
| chr19 | 14224561 | 14224681 | 120 | chr19:14224561-14224681 |
| chr19 | 14224919 | 14225039 | 120 | chr19:14224919-14225039 |
| chr19 | 15563977 | 15564097 | 120 | chr19:15563977-15564097 |
| chr19 | 17580006 | 17580126 | 120 | chr19:17580006-17580126 |
| chr19 | 17580124 | 17580244 | 120 | chr19:17580124-17580244 |
| chr19 | 20111224 | 20111344 | 120 | chr19:20111224-20111344 |
| chr19 | 20111807 | 20111927 | 120 | chr19:20111807-20111927 |
| chr19 | 29489154 | 29489274 | 120 | chr19:29489154-29489274 |
| chr19 | 29489378 | 29489498 | 120 | chr19:29489378-29489498 |
| chr19 | 29489490 | 29489610 | 120 | chr19:29489490-29489610 |
| chr19 | 30562250 | 30562370 | 120 | chr19:30562250-30562370 |
| chr19 | 30562354 | 30562474 | 120 | chr19:30562354-30562474 |
| chr19 | 30562458 | 30562578 | 120 | chr19:30562458-30562578 |
| chr19 | 30562561 | 30562681 | 120 | chr19:30562561-30562681 |
| chr19 | 31125496 | 31125616 | 120 | chr19:31125496-31125616 |
| chr19 | 31125579 | 31125699 | 120 | chr19:31125579-31125699 |
| chr19 | 31125664 | 31125784 | 120 | chr19:31125664-31125784 |
| chr19 | 36233375 | 36233495 | 120 | chr19:36233375-36233495 |
| chr19 | 36523534 | 36523654 | 120 | chr19:36523534-36523654 |
| chr19 | 38747141 | 38747261 | 120 | chr19:38747141-38747261 |
| chr19 | 41316635 | 41316755 | 120 | chr19:41316635-41316755 |
| chr19 | 43969590 | 43969710 | 120 | chr19:43969590-43969710 |
| chr19 | 43979281 | 43979401 | 120 | chr19:43979281-43979401 |
| chr19 | 47614560 | 47614680 | 120 | chr19:47614560-47614680 |
| chr19 | 48858030 | 48858150 | 120 | chr19:48858030-48858150 |
| chr19 | 49127437 | 49127557 | 120 | chr19:49127437-49127557 |
| chr19 | 49127549 | 49127669 | 120 | chr19:49127549-49127669 |
| chr19 | 49127659 | 49127779 | 120 | chr19:49127659-49127779 |
| chr19 | 49379067 | 49379187 | 120 | chr19:49379067-49379187 |
| chr19 | 49577146 | 49577266 | 120 | chr19:49577146-49577266 |
| chr19 | 51228269 | 51228389 | 120 | chr19:51228269-51228389 |
| chr19 | 51228412 | 51228532 | 120 | chr19:51228412-51228532 |
| chr19 | 51228558 | 51228678 | 120 | chr19:51228558-51228678 |
| chr19 | 52249111 | 52249231 | 120 | chr19:52249111-52249231 |
| chr19 | 52452257 | 52452377 | 120 | chr19:52452257-52452377 |
| chr19 | 53496678 | 53496798 | 120 | chr19:53496678-53496798 |
| chr19 | 55357028 | 55357148 | 120 | chr19:55357028-55357148 |
| chr19 | 55526148 | 55526268 | 120 | chr19:55526148-55526268 |
| chr19 | 58220434 | 58220554 | 120 | chr19:58220434-58220554 |
| chr19 | 58220602 | 58220722 | 120 | chr19:58220602-58220722 |
| chr19 | 58545122 | 58545242 | 120 | chr19:58545122-58545242 |
| chr19 | 59023162 | 59023282 | 120 | chr19:59023162-59023282 |
| chr2 | 264091 | 264211 | 120 | chr2:264091-264211 |
| chr2 | 430826 | 430946 | 120 | chr2:430826-430946 |
| chr2 | 620205 | 620325 | 120 | chr2:620205-620325 |
| chr2 | 906615 | 906735 | 120 | chr2:906615-906735 |
| chr2 | 1017986 | 1018106 | 120 | chr2:1017986-1018106 |
| chr2 | 1093472 | 1093592 | 120 | chr2:1093472-1093592 |
| chr2 | 1516292 | 1516412 | 120 | chr2:1516292-1516412 |
| chr2 | 1625377 | 1625497 | 120 | chr2:1625377-1625497 |
| chr2 | 1879952 | 1880072 | 120 | chr2:1879952-1880072 |
| chr2 | 1926838 | 1926958 | 120 | chr2:1926838-1926958 |
| chr2 | 1940931 | 1941051 | 120 | chr2:1940931-1941051 |
| chr2 | 4050707 | 4050827 | 120 | chr2:4050707-4050827 |
| chr2 | 6326829 | 6326949 | 120 | chr2:6326829-6326949 |
| chr2 | 24300098 | 24300218 | 120 | chr2:24300098-24300218 |
| chr2 | 25439050 | 25439170 | 120 | chr2:25439050-25439170 |
| chr2 | 26395398 | 26395518 | 120 | chr2:26395398-26395518 |
| chr2 | 29033720 | 29033840 | 120 | chr2:29033720-29033840 |
| chr2 | 29033837 | 29033957 | 120 | chr2:29033837-29033957 |
| chr2 | 40658831 | 40658951 | 120 | chr2:40658831-40658951 |
| chr2 | 63281009 | 63281129 | 120 | chr2:63281009-63281129 |
| chr2 | 63281079 | 63281199 | 120 | chr2:63281079-63281199 |
| chr2 | 63283907 | 63284027 | 120 | chr2:63283907-63284027 |
| chr2 | 63284006 | 63284126 | 120 | chr2:63284006-63284126 |
| chr2 | 63285989 | 63286109 | 120 | chr2:63285989-63286109 |
| chr2 | 69026979 | 69027099 | 120 | chr2:69026979-69027099 |
| chr2 | 81422303 | 81422423 | 120 | chr2:81422303-81422423 |
| chr2 | 91923773 | 91923893 | 120 | chr2:91923773-91923893 |
| chr2 | 101086696 | 101086816 | 120 | chr2:101086696-101086816 |
| chr2 | 109826047 | 109826167 | 120 | chr2:109826047-109826167 |
| chr2 | 113931462 | 113931582 | 120 | chr2:113931462-113931582 |
| chr2 | 119699622 | 119699742 | 120 | chr2:119699622-119699742 |
| chr2 | 119980523 | 119980643 | 120 | chr2:119980523-119980643 |
| chr2 | 127783108 | 127783228 | 120 | chr2:127783108-127783228 |
| chr2 | 131129507 | 131129627 | 120 | chr2:131129507-131129627 |
| chr2 | 135272783 | 135272903 | 120 | chr2:135272783-135272903 |
| chr2 | 166535648 | 166535768 | 120 | chr2:166535648-166535768 |
| chr2 | 175462480 | 175462600 | 120 | chr2:175462480-175462600 |
| chr2 | 192711845 | 192711965 | 120 | chr2:192711845-192711965 |
| chr2 | 200327274 | 200327394 | 120 | chr2:200327274-200327394 |
| chr2 | 208989188 | 208989308 | 120 | chr2:208989188-208989308 |
| chr2 | 210737644 | 210737764 | 120 | chr2:210737644-210737764 |
| chr2 | 215675458 | 215675578 | 120 | chr2:215675458-215675578 |
| chr2 | 221320441 | 221320561 | 120 | chr2:221320441-221320561 |
| chr2 | 222320476 | 222320596 | 120 | chr2:222320476-222320596 |
| chr2 | 228882280 | 228882400 | 120 | chr2:228882280-228882400 |
| chr2 | 235406223 | 235406343 | 120 | chr2:235406223-235406343 |
| chr2 | 238280454 | 238280574 | 120 | chr2:238280454-238280574 |
| chr2 | 238767972 | 238768092 | 120 | chr2:238767972-238768092 |
| chr2 | 241567303 | 241567423 | 120 | chr2:241567303-241567423 |
| chr2 | 241567775 | 241567895 | 120 | chr2:241567775-241567895 |
| chr2 | 241568820 | 241568940 | 120 | chr2:241568820-241568940 |
| chr2 | 241569632 | 241569752 | 120 | chr2:241569632-241569752 |
| chr2 | 241569786 | 241569906 | 120 | chr2:241569786-241569906 |
| chr2 | 241570075 | 241570195 | 120 | chr2:241570075-241570195 |
| chr2 | 241570281 | 241570401 | 120 | chr2:241570281-241570401 |
| chr20 | 3023358 | 3023478 | 120 | chr20:3023358-3023478 |
| chr20 | 20177265 | 20177385 | 120 | chr20:20177265-20177385 |
| chr20 | 25027003 | 25027123 | 120 | chr20:25027003-25027123 |
| chr20 | 29956525 | 29956645 | 120 | chr20:29956525-29956645 |
| chr20 | 43108724 | 43108844 | 120 | chr20:43108724-43108844 |
| chr20 | 43108845 | 43108965 | 120 | chr20:43108845-43108965 |
| chr20 | 43108966 | 43109086 | 120 | chr20:43108966-43109086 |
| chr20 | 43109330 | 43109450 | 120 | chr20:43109330-43109450 |
| chr20 | 43109452 | 43109572 | 120 | chr20:43109452-43109572 |
| chr20 | 44313518 | 44313638 | 120 | chr20:44313518-44313638 |
| chr20 | 44539468 | 44539588 | 120 | chr20:44539468-44539588 |
| chr20 | 47363629 | 47363749 | 120 | chr20:47363629-47363749 |
| chr20 | 47363733 | 47363853 | 120 | chr20:47363733-47363853 |
| chr20 | 47363837 | 47363957 | 120 | chr20:47363837-47363957 |
| chr20 | 47363940 | 47364060 | 120 | chr20:47363940-47364060 |
| chr20 | 59601952 | 59602072 | 120 | chr20:59601952-59602072 |
| chr20 | 59832864 | 59832984 | 120 | chr20:59832864-59832984 |
| chr20 | 60500578 | 60500698 | 120 | chr20:60500578-60500698 |
| chr20 | 61930205 | 61930325 | 120 | chr20:61930205-61930325 |
| chr20 | 62033503 | 62033623 | 120 | chr20:62033503-62033623 |
| chr20 | 62048809 | 62048929 | 120 | chr20:62048809-62048929 |
| chr20 | 62097621 | 62097741 | 120 | chr20:62097621-62097741 |
| chr20 | 62375148 | 62375268 | 120 | chr20:62375148-62375268 |
| chr21 | 34755621 | 34755741 | 120 | chr21:34755621-34755741 |
| chr21 | 36421407 | 36421527 | 120 | chr21:36421407-36421527 |
| chr21 | 38937578 | 38937698 | 120 | chr21:38937578-38937698 |
| chr21 | 41550754 | 41550874 | 120 | chr21:41550754-41550874 |
| chr21 | 47292193 | 47292313 | 120 | chr21:47292193-47292313 |
| chr22 | 18527713 | 18527833 | 120 | chr22:18527713-18527833 |
| chr22 | 37813038 | 37813158 | 120 | chr22:37813038-37813158 |
| chr22 | 38506652 | 38506772 | 120 | chr22:38506652-38506772 |
| chr22 | 40859438 | 40859558 | 120 | chr22:40859438-40859558 |
| chr22 | 45631322 | 45631442 | 120 | chr22:45631322-45631442 |
| chr22 | 46526763 | 46526883 | 120 | chr22:46526763-46526883 |
| chr22 | 46526865 | 46526985 | 120 | chr22:46526865-46526985 |
| chr22 | 46526968 | 46527088 | 120 | chr22:46526968-46527088 |
| chr22 | 46527070 | 46527190 | 120 | chr22:46527070-46527190 |
| chr22 | 48640676 | 48640796 | 120 | chr22:48640676-48640796 |
| chr22 | 48640750 | 48640870 | 120 | chr22:48640750-48640870 |
| chr22 | 48640824 | 48640944 | 120 | chr22:48640824-48640944 |
| chr22 | 48792299 | 48792419 | 120 | chr22:48792299-48792419 |
| chr22 | 48792365 | 48792485 | 120 | chr22:48792365-48792485 |
| chr22 | 48792431 | 48792551 | 120 | chr22:48792431-48792551 |
| chr22 | 48793012 | 48793132 | 120 | chr22:48793012-48793132 |
| chr22 | 48793117 | 48793237 | 120 | chr22:48793117-48793237 |
| chr22 | 48793209 | 48793329 | 120 | chr22:48793209-48793329 |
| chr22 | 48793437 | 48793557 | 120 | chr22:48793437-48793557 |
| chr22 | 48793543 | 48793663 | 120 | chr22:48793543-48793663 |
| chr22 | 48793650 | 48793770 | 120 | chr22:48793650-48793770 |
| chr22 | 49262053 | 49262173 | 120 | chr22:49262053-49262173 |
| chr22 | 49262157 | 49262277 | 120 | chr22:49262157-49262277 |
| chr22 | 50644408 | 50644528 | 120 | chr22:50644408-50644528 |
| chr22 | 50644695 | 50644815 | 120 | chr22:50644695-50644815 |
| chr22 | 51135671 | 51135791 | 120 | chr22:51135671-51135791 |
| chr3 | 13152229 | 13152349 | 120 | chr3:13152229-13152349 |
| chr3 | 16131707 | 16131827 | 120 | chr3:16131707-16131827 |
| chr3 | 21832463 | 21832583 | 120 | chr3:21832463-21832583 |
| chr3 | 37125415 | 37125535 | 120 | chr3:37125415-37125535 |
| chr3 | 39306069 | 39306189 | 120 | chr3:39306069-39306189 |
| chr3 | 49757249 | 49757369 | 120 | chr3:49757249-49757369 |
| chr3 | 50378232 | 50378352 | 120 | chr3:50378232-50378352 |
| chr3 | 50378365 | 50378485 | 120 | chr3:50378365-50378485 |
| chr3 | 50378467 | 50378587 | 120 | chr3:50378467-50378587 |
| chr3 | 54067905 | 54068025 | 120 | chr3:54067905-54068025 |
| chr3 | 118955775 | 118955895 | 120 | chr3:118955775-118955895 |
| chr3 | 126113724 | 126113844 | 120 | chr3:126113724-126113844 |
| chr3 | 130098274 | 130098394 | 120 | chr3:130098274-130098394 |
| chr3 | 136062680 | 136062800 | 120 | chr3:136062680-136062800 |
| chr3 | 170070927 | 170071047 | 120 | chr3:170070927-170071047 |
| chr3 | 180042666 | 180042786 | 120 | chr3:180042666-180042786 |
| chr3 | 183273434 | 183273554 | 120 | chr3:183273434-183273554 |
| chr3 | 183273497 | 183273617 | 120 | chr3:183273497-183273617 |
| chr3 | 183543504 | 183543624 | 120 | chr3:183543504-183543624 |
| chr3 | 197283051 | 197283171 | 120 | chr3:197283051-197283171 |
| chr4 | 720749 | 720869 | 120 | chr4:720749-720869 |
| chr4 | 1538465 | 1538585 | 120 | chr4:1538465-1538585 |
| chr4 | 3371506 | 3371626 | 120 | chr4:3371506-3371626 |
| chr4 | 4406140 | 4406260 | 120 | chr4:4406140-4406260 |
| chr4 | 19457103 | 19457223 | 120 | chr4:19457103-19457223 |
| chr4 | 38673077 | 38673197 | 120 | chr4:38673077-38673197 |
| chr4 | 43032247 | 43032367 | 120 | chr4:43032247-43032367 |
| chr4 | 62450050 | 62450170 | 120 | chr4:62450050-62450170 |
| chr4 | 79863379 | 79863499 | 120 | chr4:79863379-79863499 |
| chr4 | 84035878 | 84035998 | 120 | chr4:84035878-84035998 |
| chr4 | 88896574 | 88896694 | 120 | chr4:88896574-88896694 |
| chr4 | 88896849 | 88896969 | 120 | chr4:88896849-88896969 |
| chr4 | 99273692 | 99273812 | 120 | chr4:99273692-99273812 |
| chr4 | 99580470 | 99580590 | 120 | chr4:99580470-99580590 |
| chr4 | 135248195 | 135248315 | 120 | chr4:135248195-135248315 |
| chr4 | 156297882 | 156298002 | 120 | chr4:156297882-156298002 |
| chr4 | 187650241 | 187650361 | 120 | chr4:187650241-187650361 |
| chr5 | 95481 | 95601 | 120 | chr5:95481-95601 |
| chr5 | 143103 | 143223 | 120 | chr5:143103-143223 |
| chr5 | 1363839 | 1363959 | 120 | chr5:1363839-1363959 |
| chr5 | 1394573 | 1394693 | 120 | chr5:1394573-1394693 |
| chr5 | 1865441 | 1865561 | 120 | chr5:1865441-1865561 |
| chr5 | 1974799 | 1974919 | 120 | chr5:1974799-1974919 |
| chr5 | 2094972 | 2095092 | 120 | chr5:2094972-2095092 |
| chr5 | 2095321 | 2095441 | 120 | chr5:2095321-2095441 |
| chr5 | 2128916 | 2129036 | 120 | chr5:2128916-2129036 |
| chr5 | 2141713 | 2141833 | 120 | chr5:2141713-2141833 |
| chr5 | 2175269 | 2175389 | 120 | chr5:2175269-2175389 |
| chr5 | 2186796 | 2186916 | 120 | chr5:2186796-2186916 |
| chr5 | 2239417 | 2239537 | 120 | chr5:2239417-2239537 |
| chr5 | 2442898 | 2443018 | 120 | chr5:2442898-2443018 |
| chr5 | 2515648 | 2515768 | 120 | chr5:2515648-2515768 |
| chr5 | 2633553 | 2633673 | 120 | chr5:2633553-2633673 |
| chr5 | 2645802 | 2645922 | 120 | chr5:2645802-2645922 |
| chr5 | 2658948 | 2659068 | 120 | chr5:2658948-2659068 |
| chr5 | 2702359 | 2702479 | 120 | chr5:2702359-2702479 |
| chr5 | 2763356 | 2763476 | 120 | chr5:2763356-2763476 |
| chr5 | 3225862 | 3225982 | 120 | chr5:3225862-3225982 |
| chr5 | 3311253 | 3311373 | 120 | chr5:3311253-3311373 |
| chr5 | 3339521 | 3339641 | 120 | chr5:3339521-3339641 |
| chr5 | 3535913 | 3536033 | 120 | chr5:3535913-3536033 |
| chr5 | 3761166 | 3761286 | 120 | chr5:3761166-3761286 |
| chr5 | 4116447 | 4116567 | 120 | chr5:4116447-4116567 |
| chr5 | 4399560 | 4399680 | 120 | chr5:4399560-4399680 |
| chr5 | 7825253 | 7825373 | 120 | chr5:7825253-7825373 |
| chr5 | 7850143 | 7850263 | 120 | chr5:7850143-7850263 |
| chr5 | 9738588 | 9738708 | 120 | chr5:9738588-9738708 |
| chr5 | 17275723 | 17275843 | 120 | chr5:17275723-17275843 |
| chr5 | 17631983 | 17632103 | 120 | chr5:17631983-17632103 |
| chr5 | 19886422 | 19886542 | 120 | chr5:19886422-19886542 |
| chr5 | 26669244 | 26669364 | 120 | chr5:26669244-26669364 |
| chr5 | 40681077 | 40681197 | 120 | chr5:40681077-40681197 |
| chr5 | 52327589 | 52327709 | 120 | chr5:52327589-52327709 |
| chr5 | 60624169 | 60624289 | 120 | chr5:60624169-60624289 |
| chr5 | 80922477 | 80922597 | 120 | chr5:80922477-80922597 |
| chr5 | 112073356 | 112073476 | 120 | chr5:112073356-112073476 |
| chr5 | 112073553 | 112073673 | 120 | chr5:112073553-112073673 |
| chr5 | 114110507 | 114110627 | 120 | chr5:114110507-114110627 |
| chr5 | 134363817 | 134363937 | 120 | chr5:134363817-134363937 |
| chr5 | 137990183 | 137990303 | 120 | chr5:137990183-137990303 |
| chr5 | 140985415 | 140985535 | 120 | chr5:140985415-140985535 |
| chr5 | 150017144 | 150017264 | 120 | chr5:150017144-150017264 |
| chr5 | 164556914 | 164557034 | 120 | chr5:164556914-164557034 |
| chr5 | 170736329 | 170736449 | 120 | chr5:170736329-170736449 |
| chr5 | 171538497 | 171538617 | 120 | chr5:171538497-171538617 |
| chr5 | 176829592 | 176829712 | 120 | chr5:176829592-176829712 |
| chr6 | 1620627 | 1620747 | 120 | chr6:1620627-1620747 |
| chr6 | 3023818 | 3023938 | 120 | chr6:3023818-3023938 |
| chr6 | 4482930 | 4483050 | 120 | chr6:4482930-4483050 |
| chr6 | 11975889 | 11976009 | 120 | chr6:11975889-11976009 |
| chr6 | 11975964 | 11976084 | 120 | chr6:11975964-11976084 |
| chr6 | 12701813 | 12701933 | 120 | chr6:12701813-12701933 |
| chr6 | 12718470 | 12718590 | 120 | chr6:12718470-12718590 |
| chr6 | 13408808 | 13408928 | 120 | chr6:13408808-13408928 |
| chr6 | 14284138 | 14284258 | 120 | chr6:14284138-14284258 |
| chr6 | 16729550 | 16729670 | 120 | chr6:16729550-16729670 |
| chr6 | 19734647 | 19734767 | 120 | chr6:19734647-19734767 |
| chr6 | 26240860 | 26240980 | 120 | chr6:26240860-26240980 |
| chr6 | 26250287 | 26250407 | 120 | chr6:26250287-26250407 |
| chr6 | 26271527 | 26271647 | 120 | chr6:26271527-26271647 |
| chr6 | 27782994 | 27783114 | 120 | chr6:27782994-27783114 |
| chr6 | 28956268 | 28956388 | 120 | chr6:28956268-28956388 |
| chr6 | 31527829 | 31527949 | 120 | chr6:31527829-31527949 |
| chr6 | 32116798 | 32116918 | 120 | chr6:32116798-32116918 |
| chr6 | 32909337 | 32909457 | 120 | chr6:32909337-32909457 |
| chr6 | 41528416 | 41528536 | 120 | chr6:41528416-41528536 |
| chr6 | 41978211 | 41978331 | 120 | chr6:41978211-41978331 |
| chr6 | 42738907 | 42739027 | 120 | chr6:42738907-42739027 |
| chr6 | 43612920 | 43613040 | 120 | chr6:43612920-43613040 |
| chr6 | 50695419 | 50695539 | 120 | chr6:50695419-50695539 |
| chr6 | 56819372 | 56819492 | 120 | chr6:56819372-56819492 |
| chr6 | 106442260 | 106442380 | 120 | chr6:106442260-106442380 |
| chr6 | 116691803 | 116691923 | 120 | chr6:116691803-116691923 |
| chr6 | 149806279 | 149806399 | 120 | chr6:149806279-149806399 |
| chr6 | 151325582 | 151325702 | 120 | chr6:151325582-151325702 |
| chr6 | 151373222 | 151373342 | 120 | chr6:151373222-151373342 |
| chr6 | 159240754 | 159240874 | 120 | chr6:159240754-159240874 |
| chr6 | 166260259 | 166260379 | 120 | chr6:166260259-166260379 |
| chr7 | 960351 | 960471 | 120 | chr7:960351-960471 |
| chr7 | 1004688 | 1004808 | 120 | chr7:1004688-1004808 |
| chr7 | 1293539 | 1293659 | 120 | chr7:1293539-1293659 |
| chr7 | 1303291 | 1303411 | 120 | chr7:1303291-1303411 |
| chr7 | 1423562 | 1423682 | 120 | chr7:1423562-1423682 |
| chr7 | 2959061 | 2959181 | 120 | chr7:2959061-2959181 |
| chr7 | 3096221 | 3096341 | 120 | chr7:3096221-3096341 |
| chr7 | 18906373 | 18906493 | 120 | chr7:18906373-18906493 |
| chr7 | 27136157 | 27136277 | 120 | chr7:27136157-27136277 |
| chr7 | 27136261 | 27136381 | 120 | chr7:27136261-27136381 |
| chr7 | 27136364 | 27136484 | 120 | chr7:27136364-27136484 |
| chr7 | 27208225 | 27208345 | 120 | chr7:27208225-27208345 |
| chr7 | 27232777 | 27232897 | 120 | chr7:27232777-27232897 |
| chr7 | 27233394 | 27233514 | 120 | chr7:27233394-27233514 |
| chr7 | 27260225 | 27260345 | 120 | chr7:27260225-27260345 |
| chr7 | 30265565 | 30265685 | 120 | chr7:30265565-30265685 |
| chr7 | 31379760 | 31379880 | 120 | chr7:31379760-31379880 |
| chr7 | 41854765 | 41854885 | 120 | chr7:41854765-41854885 |
| chr7 | 41956367 | 41956487 | 120 | chr7:41956367-41956487 |
| chr7 | 44080215 | 44080335 | 120 | chr7:44080215-44080335 |
| chr7 | 45018759 | 45018879 | 120 | chr7:45018759-45018879 |
| chr7 | 75057174 | 75057294 | 120 | chr7:75057174-75057294 |
| chr7 | 75956086 | 75956206 | 120 | chr7:75956086-75956206 |
| chr7 | 78024245 | 78024365 | 120 | chr7:78024245-78024365 |
| chr7 | 87935919 | 87936039 | 120 | chr7:87935919-87936039 |
| chr7 | 92412259 | 92412379 | 120 | chr7:92412259-92412379 |
| chr7 | 94034468 | 94034588 | 120 | chr7:94034468-94034588 |
| chr7 | 100881782 | 100881902 | 120 | chr7:100881782-100881902 |
| chr7 | 100881926 | 100882046 | 120 | chr7:100881926-100882046 |
| chr7 | 101558340 | 101558460 | 120 | chr7:101558340-101558460 |
| chr7 | 101558537 | 101558657 | 120 | chr7:101558537-101558657 |
| chr7 | 117119322 | 117119442 | 120 | chr7:117119322-117119442 |
| chr7 | 124632941 | 124633061 | 120 | chr7:124632941-124633061 |
| chr7 | 126698284 | 126698404 | 120 | chr7:126698284-126698404 |
| chr7 | 126844936 | 126845056 | 120 | chr7:126844936-126845056 |
| chr7 | 126845053 | 126845173 | 120 | chr7:126845053-126845173 |
| chr7 | 126845408 | 126845528 | 120 | chr7:126845408-126845528 |
| chr7 | 126845641 | 126845761 | 120 | chr7:126845641-126845761 |
| chr7 | 126845762 | 126845882 | 120 | chr7:126845762-126845882 |
| chr7 | 126845998 | 126846118 | 120 | chr7:126845998-126846118 |
| chr7 | 126846235 | 126846355 | 120 | chr7:126846235-126846355 |
| chr7 | 126846353 | 126846473 | 120 | chr7:126846353-126846473 |
| chr7 | 126846586 | 126846706 | 120 | chr7:126846586-126846706 |
| chr7 | 127671134 | 127671254 | 120 | chr7:127671134-127671254 |
| chr7 | 127672014 | 127672134 | 120 | chr7:127672014-127672134 |
| chr7 | 127672050 | 127672170 | 120 | chr7:127672050-127672170 |
| chr7 | 127672175 | 127672295 | 120 | chr7:127672175-127672295 |
| chr7 | 131156494 | 131156614 | 120 | chr7:131156494-131156614 |
| chr7 | 134143859 | 134143979 | 120 | chr7:134143859-134143979 |
| chr7 | 136575300 | 136575420 | 120 | chr7:136575300-136575420 |
| chr7 | 154087915 | 154088035 | 120 | chr7:154087915-154088035 |
| chr7 | 154428972 | 154429092 | 120 | chr7:154428972-154429092 |
| chr7 | 154474053 | 154474173 | 120 | chr7:154474053-154474173 |
| chr7 | 154545041 | 154545161 | 120 | chr7:154545041-154545161 |
| chr7 | 157319694 | 157319814 | 120 | chr7:157319694-157319814 |
| chr7 | 157340330 | 157340450 | 120 | chr7:157340330-157340450 |
| chr7 | 157345599 | 157345719 | 120 | chr7:157345599-157345719 |
| chr7 | 157352918 | 157353038 | 120 | chr7:157352918-157353038 |
| chr7 | 157357827 | 157357947 | 120 | chr7:157357827-157357947 |
| chr7 | 157367099 | 157367219 | 120 | chr7:157367099-157367219 |
| chr7 | 157444179 | 157444299 | 120 | chr7:157444179-157444299 |
| chr7 | 157513848 | 157513968 | 120 | chr7:157513848-157513968 |
| chr7 | 157523348 | 157523468 | 120 | chr7:157523348-157523468 |
| chr7 | 157528384 | 157528504 | 120 | chr7:157528384-157528504 |
| chr7 | 157605624 | 157605744 | 120 | chr7:157605624-157605744 |
| chr7 | 157688444 | 157688564 | 120 | chr7:157688444-157688564 |
| chr7 | 157690079 | 157690199 | 120 | chr7:157690079-157690199 |
| chr7 | 157691481 | 157691601 | 120 | chr7:157691481-157691601 |
| chr7 | 157738185 | 157738305 | 120 | chr7:157738185-157738305 |
| chr7 | 157744256 | 157744376 | 120 | chr7:157744256-157744376 |
| chr7 | 157834786 | 157834906 | 120 | chr7:157834786-157834906 |
| chr7 | 157845205 | 157845325 | 120 | chr7:157845205-157845325 |
| chr7 | 157854712 | 157854832 | 120 | chr7:157854712-157854832 |
| chr7 | 157967866 | 157967986 | 120 | chr7:157967866-157967986 |
| chr7 | 158034721 | 158034841 | 120 | chr7:158034721-158034841 |
| chr7 | 158045943 | 158046063 | 120 | chr7:158045943-158046063 |
| chr7 | 158046134 | 158046254 | 120 | chr7:158046134-158046254 |
| chr7 | 158075645 | 158075765 | 120 | chr7:158075645-158075765 |
| chr8 | 2112332 | 2112452 | 120 | chr8:2112332-2112452 |
| chr8 | 2418460 | 2418580 | 120 | chr8:2418460-2418580 |
| chr8 | 3047476 | 3047596 | 120 | chr8:3047476-3047596 |
| chr8 | 20375518 | 20375638 | 120 | chr8:20375518-20375638 |
| chr8 | 27183056 | 27183176 | 120 | chr8:27183056-27183176 |
| chr8 | 27183272 | 27183392 | 120 | chr8:27183272-27183392 |
| chr8 | 42547581 | 42547701 | 120 | chr8:42547581-42547701 |
| chr8 | 53851091 | 53851211 | 120 | chr8:53851091-53851211 |
| chr8 | 56436711 | 56436831 | 120 | chr8:56436711-56436831 |
| chr8 | 57390668 | 57390788 | 120 | chr8:57390668-57390788 |
| chr8 | 59058588 | 59058708 | 120 | chr8:59058588-59058708 |
| chr8 | 61821382 | 61821502 | 120 | chr8:61821382-61821502 |
| chr8 | 67873739 | 67873859 | 120 | chr8:67873739-67873859 |
| chr8 | 67874118 | 67874238 | 120 | chr8:67874118-67874238 |
| chr8 | 67874306 | 67874426 | 120 | chr8:67874306-67874426 |
| chr8 | 69702366 | 69702486 | 120 | chr8:69702366-69702486 |
| chr8 | 70984139 | 70984259 | 120 | chr8:70984139-70984259 |
| chr8 | 89241572 | 89241692 | 120 | chr8:89241572-89241692 |
| chr8 | 90624067 | 90624187 | 120 | chr8:90624067-90624187 |
| chr8 | 93654843 | 93654963 | 120 | chr8:93654843-93654963 |
| chr8 | 96193881 | 96194001 | 120 | chr8:96193881-96194001 |
| chr8 | 98289842 | 98289962 | 120 | chr8:98289842-98289962 |
| chr8 | 98289996 | 98290116 | 120 | chr8:98289996-98290116 |
| chr8 | 98290088 | 98290208 | 120 | chr8:98290088-98290208 |
| chr8 | 98290169 | 98290289 | 120 | chr8:98290169-98290289 |
| chr8 | 99440219 | 99440339 | 120 | chr8:99440219-99440339 |
| chr8 | 99959370 | 99959490 | 120 | chr8:99959370-99959490 |
| chr8 | 102504421 | 102504541 | 120 | chr8:102504421-102504541 |
| chr8 | 104512259 | 104512379 | 120 | chr8:104512259-104512379 |
| chr8 | 104512805 | 104512925 | 120 | chr8:104512805-104512925 |
| chr8 | 104512872 | 104512992 | 120 | chr8:104512872-104512992 |
| chr8 | 104512940 | 104513060 | 120 | chr8:104512940-104513060 |
| chr8 | 117954094 | 117954214 | 120 | chr8:117954094-117954214 |
| chr8 | 118146358 | 118146478 | 120 | chr8:118146358-118146478 |
| chr8 | 119208426 | 119208546 | 120 | chr8:119208426-119208546 |
| chr8 | 134232904 | 134233024 | 120 | chr8:134232904-134233024 |
| chr8 | 134361448 | 134361568 | 120 | chr8:134361448-134361568 |
| chr8 | 141607184 | 141607304 | 120 | chr8:141607184-141607304 |
| chr8 | 142819460 | 142819580 | 120 | chr8:142819460-142819580 |
| chr8 | 143221439 | 143221559 | 120 | chr8:143221439-143221559 |
| chr8 | 143852527 | 143852647 | 120 | chr8:143852527-143852647 |
| chr8 | 143882585 | 143882705 | 120 | chr8:143882585-143882705 |
| chr8 | 145806213 | 145806333 | 120 | chr8:145806213-145806333 |
| chr9 | 35844789 | 35844909 | 120 | chr9:35844789-35844909 |
| chr9 | 96622275 | 96622395 | 120 | chr9:96622275-96622395 |
| chr9 | 99449285 | 99449405 | 120 | chr9:99449285-99449405 |
| chr9 | 100616547 | 100616667 | 120 | chr9:100616547-100616667 |
| chr9 | 119976862 | 119976982 | 120 | chr9:119976862-119976982 |
| chr9 | 137331316 | 137331436 | 120 | chr9:137331316-137331436 |
| chr9 | 137331336 | 137331456 | 120 | chr9:137331336-137331456 |
| chr9 | 138416330 | 138416450 | 120 | chr9:138416330-138416450 |
| chr9 | 139872335 | 139872455 | 120 | chr9:139872335-139872455 |
| chr9 | 139920029 | 139920149 | 120 | chr9:139920029-139920149 |
| chr9 | 139920149 | 139920269 | 120 | chr9:139920149-139920269 |
| chr9 | 140051145 | 140051265 | 120 | chr9:140051145-140051265 |

TABLE 3
Potential markers for model(s).

| Chr# | Start | Stop | Length | Position |
|---|---|---|---|---|
| chr1 | 10134550 | 10134670 | 120 | chr1: 10134550-10134670 |
| chr1 | 195732593 | 195732713 | 120 | chr1: 195732593-195732713 |
| chr11 | 117748176 | 117748296 | 120 | chr11: 117748176-117748296 |
| chr14 | 100532734 | 100532854 | 120 | chr14: 100532734-100532854 |
| chr15 | 55569455 | 55569575 | 120 | chr15: 55569455-55569575 |
| chr16 | 88202979 | 88203099 | 120 | chr16: 88202979-88203099 |
| chr17 | 2863767 | 2863887 | 120 | chr17: 2863767-2863887 |
| chr18 | 77770320 | 77770440 | 120 | chr18: 77770320-77770440 |
| chr18 | 77770425 | 77770545 | 120 | chr18: 77770425-77770545 |
| chr18 | 77770531 | 77770651 | 120 | chr18: 77770531-77770651 |
| chr19 | 2723033 | 2723153 | 120 | chr19: 2723033-2723153 |
| chr19 | 2723141 | 2723261 | 120 | chr19: 2723141-2723261 |
| chr19 | 38042412 | 38042532 | 120 | chr19: 38042412-38042532 |
| chr19 | 48857934 | 48858054 | 120 | chr19: 48857934-48858054 |
| chr5 | 4629157 | 4629277 | 120 | chr5: 4629157-4629277 |
| chr5 | 134364327 | 134364447 | 120 | chr5: 134364327-134364447 |
| chr5 | 176829688 | 176829808 | 120 | chr5: 176829688-176829808 |
| chr6 | 4352275 | 4352395 | 120 | chr6: 4352275-4352395 |
| chr6 | 4352395 | 4352515 | 120 | chr6: 4352395-4352515 |
| chr6 | 34071136 | 34071256 | 120 | chr6: 34071136-34071256 |
| chr6 | 34071209 | 34071329 | 120 | chr6: 34071209-34071329 |
| chr7 | 73641028 | 73641148 | 120 | chr7: 73641028-73641148 |
| chr7 | 157319146 | 157319266 | 120 | chr7: 157319146-157319266 |
| chr7 | 157563542 | 157563662 | 120 | chr7: 157563542-157563662 |
| chr8 | 142852803 | 142852923 | 120 | chr8: 142852803-142852923 |
| chr8 | 142852878 | 142852998 | 120 | chr8: 142852878-142852998 |

TABLE 4
Markers with stable methylation values.

| Chr# | Start | Stop | Length | Position |
|---|---|---|---|---|
| chr19 | 45909702 | 45909822 | 120 | chr19: 45909702-45909822 |
| chr6 | 91297130 | 91297250 | 120 | chr6: 91297130-91297250 |
| chr7 | 75932502 | 75932622 | 120 | chr7: 75932502-75932622 |
| chr13 | 43566253 | 43566373 | 120 | chr13: 43566253-43566373 |
| chr14 | 106410867 | 106410987 | 120 | chr14: 106410867-106410987 |
| chr6 | 73329928 | 73330048 | 120 | chr6: 73329928-73330048 |
| chr9 | 33166677 | 33166797 | 120 | chr9: 33166677-33166797 |

TABLE 5
Endogenous Controls List.

| Chr# | Start | Stop | Length | Position |
|---|---|---|---|---|
| chr19 | 12305802 | 12305922 | 120 | chr19: 12305802-12305922 |
| chr7 | 27232777 | 27232897 | 120 | chr7: 27232777-27232897 |
| chr5 | 112073553 | 112073673 | 120 | chr5: 112073553-112073673 |
| chr6 | 42738907 | 42739027 | 120 | chr6: 42738907-42739027 |
| chr1 | 145395693 | 145395813 | 120 | chr1: 145395693-145395813 |
| chr4 | 88896849 | 88896969 | 120 | chr4: 88896849-88896969 |
| chr8 | 99959370 | 99959490 | 120 | chr8: 99959370-99959490 |
| chr1 | 92150444 | 92150564 | 120 | chr1: 92150444-92150564 |
| chr8 | 59058588 | 59058708 | 120 | chr8: 59058588-59058708 |
| chr6 | 31527829 | 31527949 | 120 | chr6: 31527829-31527949 |
| chr1 | 26872458 | 26872578 | 120 | chr1: 26872458-26872578 |
| chr17 | 6899237 | 6899357 | 120 | chr17: 6899237-6899357 |
| chr1 | 206753393 | 206753513 | 120 | chr1: 206753393-206753513 |
| chr22 | 45631322 | 45631442 | 120 | chr22: 45631322-45631442 |
| chr21 | 36421407 | 36421527 | 120 | chr21: 36421407-36421527 |
| chr17 | 80358859 | 80358979 | 120 | chr17: 80358859-80358979 |
| chr1 | 230285939 | 230286059 | 120 | chr1: 230285939-230286059 |
| chr16 | 29757258 | 29757378 | 120 | chr16: 29757258-29757378 |
| chr13 | 20805320 | 20805440 | 120 | chr13: 20805320-20805440 |
| chr4 | 720749 | 720869 | 120 | chr4: 720749-720869 |
| chr17 | 48545890 | 48546010 | 120 | chr17: 48545890-48546010 |
| chr7 | 158046134 | 158046254 | 120 | chr7: 158046134-158046254 |
| chr10 | 103911532 | 103911652 | 120 | chr10: 103911532-103911652 |
| chr11 | 1892247 | 1892367 | 120 | chr11: 1892247-1892367 |
| chr14 | 93406022 | 93406142 | 120 | chr14: 93406022-93406142 |
| chr9 | 139920029 | 139920149 | 120 | chr9: 139920029-139920149 |
| chr19 | 15563977 | 15564097 | 120 | chr19: 15563977-15564097 |
| chr12 | 5211801 | 5211921 | 120 | chr12: 5211801-5211921 |
| chr4 | 93103491 | 93103611 | 120 | chr4: 93103491-93103611 |
| chr18 | 3879243 | 3879363 | 120 | chr18: 3879243-3879363 |
| chr18 | 3879243 | 3879363 | 120 | chr18: 3879243-3879363 |
| chr17 | 78999580 | 78999700 | 120 | chr17: 78999580-78999700 |
| chr10 | 88683960 | 88684080 | 120 | chr10: 88683960-88684080 |
| chr18 | 61143866 | 61143986 | 120 | chr18: 61143866-61143986 |
| chr13 | 113977272 | 113977392 | 120 | chr13: 113977272-113977392 |
| chr1 | 151660727 | 151660847 | 120 | chr1: 151660727-151660847 |

TABLE 6
Markers selected for model v1.2.

| cpg_id | marker_id | Origin | Probe | Gene |
|---|---|---|---|---|
| chr1_10134610_10134611 | HCV_pos_group_1_marker_1 | KZ | chr1: 10134550- | UBE4 |
| | | | 10134670 | B |
| chr1_10134620_10134621 | HCV_pos_group_1_marker_1 | KZ | chr1: 10134550- | UBE4 |
| | | | 10134670 | B |
| chr1_151129298_151129299 | marker hcv_neg_1-YZ3 | KZ | chr1: 151129238- | TNFAI |
| | | | 151129358 | P8L2 |
| | | | | (SCN |
| | | | | M1) |
| chr1_206753453_206753454 | HCV_pos_group_1_marker_40 | KZ | chr1: 206753393- | RASS |
| | | | 206753513 | F5 |
| chr1_26872518_26872519 | HCV_pos_group_1_marker_48 | KZ | chr1: 26872458- | RPS6K |
| | | | 26872578 | A1 |
| chr1_26872525_26872526 | HCV_pos_group_1_marker_48 | KZ | chr1: 26872458- | RPS6K |
| | | | 26872578 | A1 |
| chr1_26872538_26872539 | HCV pos_group_1_marker_48 | KZ | chr1: 26872458- | RPS6K |
| | | | 26872578 | A1 |
| chr10_30818609_30818610 | HCV_pos_group_1_marker_58 | KZ | chr10: 30818504- | Not |
| | | | 30818624 | defined |
| chr10_30818611_30818612 | HCV_pos_group_1_marker_58 | KZ | chr10: 30818504- | Not |
| | | | 30818624 | defined |
| chr10_30818618_30818619 | HCV_pos_group_1_marker_58 | KZ | chr10: 30818504- | Not |
| | | | 30818624 | defined |
| chr11_314086_314087 | HCV_pos_group_1_marker_60 | Lit Gao et al. . Clin | chr11: 313999-314119 | IFITM |
| | | Epigenetics 2015; 7: 86. | | 1 |
| chr11_314098_314099 | HCV pos_group_1_marker_60 | Lit Gao et al. . Clin | chr11: 313999-314119 | IFITM |
| | | Epigenetics 2015; 7: 86. | | 1 |
| chr11_314106_314107 | HCV_pos_group_1_marker_60 | Lit Gao et al. . Clin | chr11: 313999-314119 | IFITM |
| | | Epigenetics 2015; 7: 86. | | 1 |
| chr11_314113_314114 | HCV_pos_group_1_marker_60 | Lit Gao et al. . Clin | chr11: 313999-314119 | IFITM |
| | | Epigenetics 2015; 7: 86. | | 1 |
| chr11_314074_314075 | marker_climb_6 | Lit Gao et al. . Clin | chr11: 313999-314119 | IFITM |
| | | Epigenetics 2015; 7: 86. | | 1 |
| chr11_314086_314087 | marker_climb_6 | Lit Gao et al. . Clin | chr11: 313999-314119 | IFITM |
| | | Epigenetics 2015; 7: 86. | | 1 |
| chr11_70211523_70211524 | HCV_pos_group_1_marker_64 | KZ | chr11: 70211458- | PPFIA |
| | | | 70211578 | 1 |
| chr11_70211531_70211532 | HCV_pos_group_1_marker_64 | KZ | chr11: 70211458- | PPFIA |
| | | | 70211578 | 1 |
| chr11_70211534_70211535 | HCV_pos_group_1_marker_64 | KZ | chr11: 70211458- | PPFIA |
| | | | 70211578 | 1 |
| chr11_70211540_70211541 | HCV_pos_group_1_marker_64 | KZ | chr11: 70211458- | PPFIA |
| | | | 70211578 | 1 |
| chr11_7326990_7326991 | CHALM_22 | ENA | chr11: 7326930- | SYT9 |
| | | | 7327050 | |
| chr14_100532790_100532791 | HCV_pos_group_1_marker_75 | KZ | chr14: 100532734- | EVL |
| | | | 100532854 | |
| chr14_100532797_100532798 | HCV_pos_group_1_marker_75 | KZ | chr14: 100532734- | EVL |
| | | | 100532854 | |
| chr16_29757323_29757324 | HCV_pos_group_1_marker_86 | KZ | chr16: 29757258- | C16orf |
| | | | 29757378 | 54 |
| chr16_29757334_29757335 | HCV_pos_group_1_marker_86 | KZ | chr16: 29757258- | C16orf |
| | | | 29757378 | 54 |
| chr16_29757344_29757345 | HCV_pos_group_1_marker_86 | KZ | chr16: 29757258- | C16orf |
| | | | 29757378 | 54 |
| chr16_29757350_29757351 | HCV_pos_group_1_marker_86 | KZ | chr16: 29757258- | C16orf |
| | | | 29757378 | 54 |
| chr16_29757360_29757361 | HCV_pos_group_1_marker_86 | KZ | chr16: 29757258- | C16orf |
| | | | 29757378 | 54 |
| chr16_29757360_29757361 | HCV_pos_group_2_marker_9 | KZ | chr16: 29757258- | C16orf |
| | | | 29757378 | 54 |
| chr16_29757375_29757376 | HCV_pos_group_2_marker_9 | KZ | chr16: 29757258- | C16orf |
| | | | 29757378 | 54 |
| chr16_29757334_29757335 | marker_climb_7 | KZ | chr16: 29757258- | C16orf |
| | | | 29757378 | 54 |
| chr16_29757344_29757345 | marker_climb_7 | KZ | chr16: 29757258- | C16orf |
| | | | 29757378 | 54 |
| chr17_57915773_57915774 | HCV_pos_group_2_marker_13 | Patent US 10,513,739 | chr17: 57915657- | VMP1 |
| | | B2, Table 9 | 57915777 | |
| chr17_57915717_57915718 | marker_hcv_neg_15 | Patent US 10,513,739 | chr17: 57915657- | VMP1 |
| | | B2, Table 9 | 57915777 | |
| chr17_80358819_80358820 | HCV pos group_1_marker_92 | KZ | chr17: 80358759- | OGFO |
| | | | 80358879 | D3 |
| chr17_80358829_80358830 | HCV_pos_group_1_marker_92 | KZ | chr17: 80358759- | OGFO |
| | | | 80358879 | D3 |
| chr17_80358847_80358848 | HCV_pos_group_1_marker_92 | KZ | chr17: 80358759- | OGFO |
| | | | 80358879 | D3 |
| chr17_80358850_80358851 | HCV pos_group_1_marker_92 | KZ | chr17: 80358759- | OGFO |
| | | | 80358879 | D3 |
| chr17_80358919_80358920 | marker_climb_4 | KZ | chr17: 80358859- | OGFO |
| | | | 80358979 | D3 |
| chr17_80358932_80358933 | marker_climb_4 | KZ | chr17: 80358859- | OGFO |
| | | | 80358979 | D3 |
| chr19_2723034_2723035 | marker_hcv_neg_42 | CHALM | chr19: 2723033- | Not |
| | | | 2723153 | defined |
| chr19_2723147_2723148 | marker_climb_3 | CHALM | chr19: 2723033- | Not |
| | | | 2723153, | defined |
| | | | chr19: 2723141- | |
| | | | 2723261 | |
| chr19_2723147_2723148 | marker_hcv_neg_42 | CHALM | chr19: 2723033- | Not |
| | | | 2723153, | defined |
| | | | chr19: 2723141- | |
| | | | 2723261 | |
| chr19_2723169_2723170 | marker_climb_3 | CHALM | chr19: 2723141- | Not |
| | | | 2723261 | defined |
| chr19_2723181_2723182 | marker_climb_3 | CHALM | chr19: 2723141- | Not |
| | | | 2723261 | defined |
| chr19_2723184_2723185 | marker_hcv_neg_19 | CHALM | chr19: 2723141- | Not |
| | | | 2723261 | defined |
| chr19_2723189_2723190 | marker_hcv_neg_19 | CHALM | chr19: 2723141- | Not |
| | | | 2723261 | defined |
| chr19_30562320_30562321 | marker_climb_8 | CHALM | chr19: 30562250- | Not |
| | | | 30562370 | defined |
| chr19_30562385_30562386 | marker_climb_climb_8 | CHALM | chr19: 30562354- | Not |
| | | | 30562474 | defined |
| chr2_113931518_113931519 | HCV_pos_group_1_marker_105 | KZ | chr2: 113931462- | PSD4 |
| | | | 113931582 | |
| chr2_113931525_113931526 | HCV_pos_group_1_marker_105 | KZ | chr2: 113931462- | PSD4 |
| | | | 113931582 | |
| chr22_45631379_45631380 | HCV_pos_group_1_marker_120 | KZ | chr22: 45631322- | KIAAO |
| | | | 45631442 | 930 |
| chr22_45631384_45631385 | HCV pos_group_1_marker_120 | KZ | chr22: 45631322- | KIAAO |
| | | | 45631442 | 930 |
| chr3_197283111_197283112 | YZ1 | KZ | chr3: 197283051- | BDH1 |
| | | | 197283171 | |
| chr5_176829755_176829756 | HCV_pos_group_1_marker_137 | Patent WO 2019- | chr5: 176829688- | F12 |
| | | 071161 - HCC p.431-2, | 176829808 | |
| | | #11 | | |
| chr5_176829777_176829778 | HCV_pos_group_1_marker_137 | Patent WO 2019- | chr5: 176829688- | F12 |
| | | 071161 - HCC p.431-2, | 176829808 | |
| | | #11 | | |
| chr6_11976024_11976025 | marker_climb_9 | KZ | chr6: 11975964- | Not |
| | | | 11976084 | defined |
| chr6_11976066_11976067 | marker_climb_9 | KZ | chr6: 11975964- | Not |
| | | | 11976084 | defined |
| chr6_26240920_26240921 | marker_climb_10 | Lit Revil et al, | chr6: 26240860- | H4C6 |
| | | GASTRO | 26240980 | |
| | | 2013; 145: 1424-1435 | | |
| chr6_26240922_26240923 | marker_climb_10 | Lit Revil et al, | chr6: 26240860- | H4C6 |
| | | GASTRO | 26240980 | |
| | | 2013; 145: 1424-1435 | | |
| chr6_26240920_26240921 | marker_hcv_neg_22 | Lit Revil et al, | chr6: 26240860- | H4C6 |
| | | GASTRO | 26240980 | |
| | | 2013; 145: 1424-1435 | | |
| chr6_26240930_26240931 | marker_hcv_neg_22 | Lit Revil et al, | chr6: 26240860- | H4C6 |
| | | GASTRO | 26240980 | |
| | | 2013; 145: 1424-1435 | | |
| chr6 26240939_26240940 | marker_hcv_neg_37 | Lit Revil et al, | chr6: 26240860- | H4C6 |
| | | GASTRO | 26240980 | |
| | | 2013; 145: 1424-1435 | | |
| chr6_26240950_26240951 | marker_hcv_neg_37 | Lit Revil et al, | chr6: 26240860- | H4C6 |
| | | GASTRO | 26240980 | |
| | | 2013; 145: 1424-1435 | | |
| chr6_26240975_26240976 | marker_hcv_neg_37 | Lit Revil et al, | chr6: 26240860- | H4C6 |
| | | GASTRO | 26240980 | |
| | | 2013; 145: 1424-1435 | | |
| chr6_31527889_31527890 | HCV_pos_group_1_marker_145 | KZ | chr6: 31527829- | LOC10 |
| | | | 31527949 | 028732 |
| | | | | 9 |
| chr6_31527893_31527894 | HCV_pos_group_1_marker_145 | KZ | chr6: 31527829- | LOC10 |
| | | | 31527949 | 028732 |
| | | | | 9 |
| chr6_31527920_31527921 | HCV_pos_group_1_marker_145 | KZ | chr6: 31527829- | LOC10 |
| | | | 31527949 | 028732 |
| | | | | 9 |
| chr6_41528491_41528492 | HCV_pos_group_1_marker_149 | KZ | chr6: 41528416- | FOXP |
| | | | 41528536 | 4 |
| | | | | (AS1) |
| chr6_41528497_41528498 | HCV_pos_group_1_marker_149 | KZ | chr6: 41528416- | FOXP |
| | | | 41528536 | 4 |
| | | | | (AS1) |
| chr6_41528499_41528500 | HCV_pos_group_1_marker_149 | KZ | chr6: 41528416- | FOXP |
| | | | 41528536 | 4 |
| | | | | (AS1) |
| chr6_41528502_41528503 | HCV_pos_group_1_marker_149 | KZ | chr6: 41528416- | FOXP |
| | | | 41528536 | 4 |
| | | | | (AS1) |
| chr7_157319199_157319200 | CHALM_16 | Lit Zheng et al. Clinical | chr7: 157319146- | Not |
| | | Epigenetics (2019) | 157319266 | defined |
| | | 11: 145 | | |
| chr7_157319203_157319204 | CHALM_16 | Lit Zheng et al. Clinical | chr7: 157319146- | Not |
| | | Epigenetics (2019) | 157319266 | defined |
| | | 11: 145 | | |
| chr7_157319206_157319207 | CHALM_16 | Lit Zheng et al. Clinical | chr7: 157319146- | Not |
| | | Epigenetics (2019) | 157319266 | defined |
| | | 11: 145 | | |
| chr7_157563602_157563603 | marker_hcv_neg_29 | XENA | chr7: 157563542- | PTPR |
| | | | 157563662 | N2 |
| chr7_45018849_45018850 | YZ2 | KZ | chr7: 45018759- | YZ2 |
| | (MYO1G) | | 45018879 | (MYO |
| | | | | 1G) |
| chr7_73641071_73641072 | marker_climb_1 | KZ | chr7: 73641028- | LAT2 |
| | | | 73641148 | |
| chr7_73641105_73641106 | marker_climb_1 | KZ | chr7: 73641028- | LAT2 |
| | | | 73641148 | |
| chr8_142852876_142852877 | CHALM_15 | Lit Zheng et al. Clinical | chr8: 142852803- | Not |
| | | Epigenetics (2019) | 142852923 | defined |
| | | 11: 145 | | |
| chr8_142852883_142852884 | CHALM_15 | Lit Zheng et al. Clinical | chr8: 142852803- | Not |
| | | Epigenetics (2019) | 142852923 | defined |
| | | 11: 145 | | |
| chr8_96193898_96193899 | marker_climb_11 | KZ | chr8: 96193881- | Not |
| | | | 96194001 | defined |
| chr8_96193941_96193942 | marker_climb_11 | KZ | chr8: 96193881- | Not |
| | | | 96194001 | defined |

TABLE 7
HCC vs. Non-HCC: ROC-AUCs by stages from different methods.
HCC vs. Non-HCC (healthy + benign + otherCancers)

| | RGC all stages | Stage I | Stage II | Stage III | Stage IV | Unstaged |
|---|---|---|---|---|---|---|
| | (N = 101) | (N = 24) | (N = 6) | (N = 29) | (N = 21) | (N = 21) |
| | nonHCC_cancer | nonHCC_stage_I | nonHCC_stage_II | nonHCC_stage_III | nonHCC_stage_IV | nonHCC_unstaged |
| Meth only | 0.898 | 0.865 | 0.964 | 0.915 | 0.925 | 0.867 |
| AFP only | 0.896 | 0.897 | 0.721 | 0.899 | 0.958 | 0.878 |
| Three-protein | 0.909 | 0.861 | 0.760 | 0.966 | 0.931 | 0.906 |
| Meth + AFP | 0.929 | 0.909 | 0.962 | 0.938 | 0.945 | 0.913 |
| Meth + Three-protein | 0.936 | 0.894 | 0.887 | 0.980 | 0.954 | 0.918 |

TABLE 8
HCC vs. Benign: ROC-AUCs by stages from different methods.
HCC vs. Benign

| | RGC all stages | Stage I | Stage II | Stage III | Stage IV | Unstaged |
|---|---|---|---|---|---|---|
| | (N = 101) | (N = 24) | (N = 6) | (N = 29) | (N = 21) | (N = 21) |
| | nonHCC_cancer | nonHCC_stage_I | nonHCC_stage_II | nonHCC_stage_III | nonHCC_stage_IV | nonHCC_unstaged |
| Meth only | 0.918 | 0.894 | 0.983 | 0.929 | 0.940 | 0.888 |
| AFP only | 0.905 | 0.907 | 0.739 | 0.908 | 0.961 | 0.890 |
| Three-protein | 0.913 | 0.867 | 0.762 | 0.968 | 0.937 | 0.910 |
| Meth + AFP | 0.938 | 0.929 | 0.985 | 0.947 | 0.948 | 0.913 |
| Meth + Three-protein | 0.942 | 0.906 | 0.895 | 0.981 | 0.958 | 0.927 |

TABLE 9
HCC vs. Non-HCC: Sensitivities at 90% specificity by stages from different methods.
HCC vs. Non-HCC (healthy + benign + otherCancers)

| | RGC all stages | Stage I | Stage II | Stage III | Stage IV | Unstaged |
|---|---|---|---|---|---|---|
| | (N = 101) | (N = 24) | (N = 6) | (N = 29) | (N = 21) | (N = 21) |
| | nonHCC_cancer | nonHCC_stage_I | nonHCC_stage_II | nonHCC_stage_III | nonHCC_stage_IV | nonHCC_unstaged |
| Meth only | 76.2% | 58.3% | 100.0% | 79.3% | 85.7% | 76.2% |
| AFP only | 77.2% | 75.0% | 50.0% | 79.3% | 85.7% | 76.2% |
| Three-protein | 79.2% | 70.8% | 66.7% | 89.7% | 81.0% | 76.2% |
| Meth + AFP | 85.1% | 79.2% | 100.0% | 86.2% | 90.5% | 81.0% |
| Meth + Three-protein | 85.1% | 75.0% | 66.7% | 96.6% | 85.7% | 85.7% |

TABLE 10
HCC vs. Benign: Sensitivities at 90% specificity by stages from different methods.
HCC vs. Benign

| | RGC all stages | Stage I | Stage II | Stage III | Stage IV | Unstaged |
|---|---|---|---|---|---|---|
| | (N = 101) | (N = 24) | (N = 6) | (N = 29) | (N = 21) | (N = 21) |
| | nonHCC_cancer | nonHCC_stage_I | nonHCC_stage_II | nonHCC_stage_III | nonHCC_stage_IV | nonHCC_unstaged |
| Meth only | 80.2% | 70.8% | 100.0% | 82.8% | 85.7% | 76.2% |
| APP only | 76.2% | 75.0% | 50.0% | 79.3% | 81.0% | 76.2% |
| Three-protein | 78.2% | 70.8% | 66.7% | 89.7% | 76.2% | 76.2% |
| Meth + APP | 87.1% | 83.3% | 100.0% | 89.7% | 90.5% | 81.0% |
| Meth + Three-protein | 85.1% | 75.0% | 66.7% | 96.6% | 85.7% | 85.7% |

### Example 4

This example demonstrates improved results obtained for a liver cancer test (HelioLiver Test) based on a biomarker profile for early detection of liver cancer, and specifically detection of hepatocellular carcinoma (HCC), as compared to other liver cancer diagnostics. As described herein, the biomarker profile included data pertaining to a methylation profile, a polypeptide profile, and a demographic profile as described herein.

Subjects recruited in this study were patients newly diagnosed with HCC or patients with a benign liver disease that were recommended for HCC surveillance and were found to be without HCC (control subjects). Subjects with HCC were diagnosed by histopathologic examination or by specific radiologic characteristics according to current practice guidelines in China. HCC stage (i.e., extent of tumor spread) was determined for subjects according to the American Joint Commission on Cancer (AJCC) 8th Edition. The control subjects were patients who were recommended to HCC surveillance in China due to underlying chronic liver disease, including chronic fibrotic liver diseases from any cause, chronic hepatitis B virus (HBV) infection, chronic hepatitis C virus infection, fatty liver disease, and nonalcoholic fatty liver disease. The presence of cirrhosis was defined by histology or clinical evidence of portal hypertension in subjects with chronic liver disease. All clinical information, including patient demographics and clinical characteristics, were prospectively obtained from medical records. All subjects were prospectively and consecutively enrolled at the Third Affiliated Hospital of Sun Yat-sen University (Guangzhou, China) and the First Affiliate Hospital of Guangzhou Medical University (Guangzhou, China) between 2020 and 2021 with written informed consent. The study was approved by their respective ethical review boards.

In total, the study included 140 patients with HCC and 150 patients diagnosed with a benign liver disease without HCC (control subjects without HCC). A total of 93 subjects were enrolled at the Third Affiliated Hospital of Sun Yat-sen University, and 210 subjects were enrolled at the First Affiliate Hospital of Guangzhou Medical University. Subsequently, 5 subjects were excluded for incomplete health and/or demographic information, and 44 subjects were excluded for failing to meet quality control criteria for the HelioLiver Test, specifying an average sequencing coverage of ≥50 times among all target sites. The final study population analyzed consisted of 122 patients with HCC and 125 control subjects (Table A).

TABLE A
Clinical characteristics of study participants.

| Subjects (n) | HCC | Control subjects |
|---|---|---|
| Age, median, years | 122 | 125 |
| | 55 | 47 |
| Sex | | |
| Male (n) | 106 (87%) | 83 (66%) |
| Female (n) | 16 (13%) | 42 (34%) |
| Liver disease | | |
| Cirrhosis, n (%) | 45 (37%) | 46 (37%) |
| Chronic HBV, n (%) | 88 (72%) | 72 (58%) |
| Chronic HCV, n (%) | 3 (3%) | 4 (3%) |
| Fatty liver disease, n (%) | 1 (1%) | 20 (16%) |
| Otherª, n (%) | 34 (28%) | 26 (21%) |
| Protein tumor markers | | |
| AFP, median (ng/mL) | AFP, median (ng/ml) | AFP, median (ng/ml) |
| 65.8 1.7 | 65.8 1.7 | 65.8 1.7 |
| AFP-L3%, median (%) | AFP-L3%, median (%) | AFP-L3%, median (%) |
| 10% <5% | 10% <5% | 10% <5% |
| DCP, median (ng/mL) | DCP, median (ng/ml) | DCP, median (ng/ml) |
| 13.8 0.6 | 13.8 0.6 | 13.8 0.6 |
| Stage | | |
| I | 29 (24%) | |
| II | 8 (7%) | |
| III | 43 (35%) | |
| IV | 28 (23%) | |
| Unstaged or unknown | 14 (12%) | |

Serum concentrations of AFP, AFP-L3%₀, and DCP were measured by using commercially available assays (Hotgen Biotech, Beijing, China) on a HotGen MQ60 instrument according to the manufacturer's instructions. The Helios Eclipse platform was used to evaluate methylation patterns of cfDNA at target sites. To this end, total cfDNA was isolated from specimens by using the EliteHealth cfDNA Extraction Kit (EliteHealth, Guangzhou Youze, China). Isolated cfDNA was eluted into nuclease-free low-bind 1.5-mL microcentrifuge tubes and stored at −80° C. DNA concentration was measured using the Qubit dsDNA High Sensitivity Assay Kit (Thermo Fisher Scientific, USA) as per manufacturer's instructions. A total of 5 ng cfDNA per sample was used to prepare the barcoded next-generation sequencing (NGS) libraries by using the NEB Next Enzymatic Methyl-seq Kit (New England Biolabs, USA) according to the manufacturer's instructions. The libraries were then pooled in groups of 24 barcoded libraries at 100 ng each and hybridized with a custom set of HelioLiver capture probes (Twist Bioscience, USA) to capture the target library sequences using the Twist Fast Hybridization and Wash Kit, along with the Twist Universal Blocker. The captured libraries were then supplemented with 20% PhiX genomic DNA library to increase base calling diversity and submitted for NGS on either a HiSeq X or a NovaSeq 6000 platform (Illumina, USA).

Raw sequencing data were first trimmed by TrimGalore (ver. 0.6.5) to remove low-quality (Phred score <20) sequences and potential adapter contamination. To remove M-bias, 5 bp and 10 bp of sequence was trimmed from the 5′ end of Read 1 and Read 2, respectively. Cleaned sequencing reads were then aligned to the hg19 human reference genome by using BSMAP (ver. 2.90). The aligned reads were further processed by Samtools (ver.1.13) and Bedtools (ver. 2.29.1) to select only primarily mapped reads with fragment size between 80 bp and 200 bp. Methratio.py (BSMAP) was finally used to extract the methylation ratio from aligned bam files. Samples with insufficient sequencing depth (<50 times) were excluded from the downstream analysis.

The HelioLiver Test was developed to discriminate between patients with HCC from high-risk patients without HCC. A preliminary NGS methylation (m)-cfDNA panel was assessed to obtain an optimized subset of m-cf-DNA markers. Then, an optimized subset of m-cfDNA markers, clinically available serum protein markers (AFP, AFP-L3%, and DCP), and patient demographics (age and sex) were combined to generate the HelioLiver Test. To this end, we first selected cytosine-guanine dinucleotide (CpG) sites showing significant methylation alteration in HCC samples compared to non-HCC control samples. Subsequently, the feature selection R package “Boruta” was used to identify the optimal cfDNA methylation markers within the Integrative Training Set. This approach identified 77 CpG sites in 28 genes (Table 11) as being significantly and consistently differentially methylated for HCC and was used to construct the cfDNA methylation model for the methylation profile. For model training, we assessed different off-the-shelf machine learning models and chose the random forest model (implemented by R package “Ranger”) that showed the best performance. The hyper parameters of the random forest model were fine-tuned by the grid-search method. The cfDNA methylation component, protein tumor marker component, and demographic component were combined by using a decision tree model to generate the HelioLiver Test diagnostic algorithm. The threshold of the HelioLiver Test diagnostic algorithm was fixed based on the out-of-bag predictions in the Training Set to achieve approximately 90% specificity. The HelioLiver diagnostic algorithm was then locked before the initiation of a validation study (ENCORE). For cfDNA methylation analysis, targeted NGS capture was performed by using the preliminary NGS m-cfDNA panel. However, only the 28 target genes (77 CpG sites) included in the HelioLiver Test were used to calculate HelioLiver Test results.

For the independent clinical validation of the HelioLiver Test, the primary endpoint was to compare the area under receiver operating characteristic (AUROC) curve of the HelioLiver Test to both AFP alone and the GALAD score. The co-secondary endpoints were to compare the sensitivity and specificity of the HelioLiver Test (using a prespecified diagnostic algorithm and cutoffs) to AFP at the most commonly reported clinical cutoff of 20 ng/mL, at a lower cutoff of 10 ng/mL, and to the GALAD score at a proposed cutoff of −0.63. As an exploratory endpoint, the sensitivity of the HelioLiver Test was compared with AFP and the GALAD score at standardized specificities. As a post hoc analysis, the performance characteristics of AFP-L3% alone, DCP alone, and the combination of AFP and DCP were also calculated for comparison. Due to the relatively high prevalence of chronic HBV within the study population, a post hoc subgroup analysis was additionally performed in a subpopulation of subjects without chronic HBV infection, to compare the AUROC curve, sensitivity, and specificity of the HelioLiver Test, AFP alone, and the GALAD score. The comparison of the AUROCs for both all subjects with HCC and only early (stage I and II) HCC were performed by sample permutation-based Wilcoxon signed-rank test (10,000 permutations) with Bonferroni correction. The comparisons of the sensitivity and specificity of the HelioLiver Test to AFP and GALAD score were performed using McNemar's test for paired proportions. A two-tailed p value less than 0.05 was regarded as statistically significant. All statistical analyses were performed by using Prism software version 8.0 (GraphPad, La Jolla, CA). To assess confounding, the logit function from the python statsmodels module (statsmodels.formula.api.logit) was used to perform logistic regression, with the cancer status as the response variable, and the HelioLiver Test result along with age, gender, and several benign liver conditions as explanatory variables. For each variable, the exponential of the coefficient was calculated to determine the odds ratio.

It was observed that 10 of the 28 genes in our cfDNA panel are involved in molecular pathways implicated in HCC pathogenesis, whereas of the 497 unselected genes from the preliminary m-cfDNA assessment, only one has been associated in molecular pathways implicated in HCC pathogenesis.

For validation of the HelioLiver, we prospectively enrolled 247 evaluable subjects, including 122 subjects diagnosed with HCC and 125 subjects with a chronic liver disease, who were found to be without HCC after undergoing HCC surveillance (control subjects). The demographic and clinical characteristics of all eligible subjects are described in Table A. The subjects with HCC were older (median age=55 years) compared with the control subjects (median age=47 years). The major disease etiology was HBV infection similarly among both subjects with HCC (72%) and control subjects (58%), in part due to the high rate of HBV infections in China. As expected, AFP, AFP-L3%, DCP, and the GALAD score were higher in the subjects with HCC compared to the control subjects.

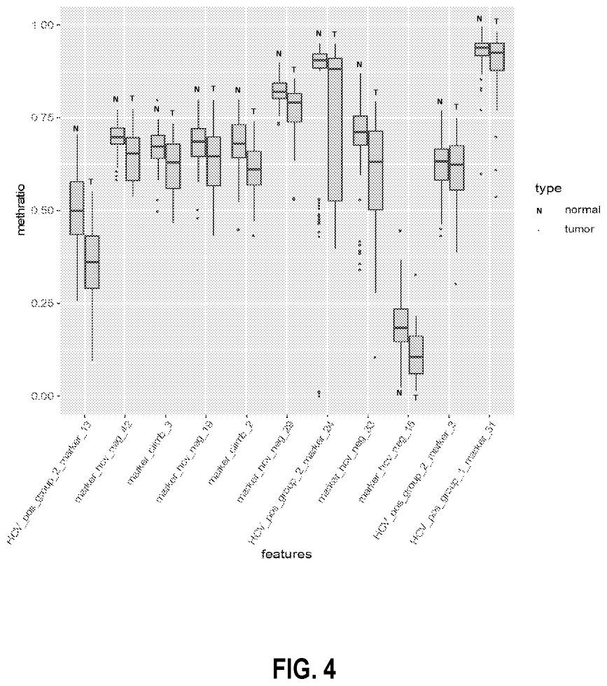

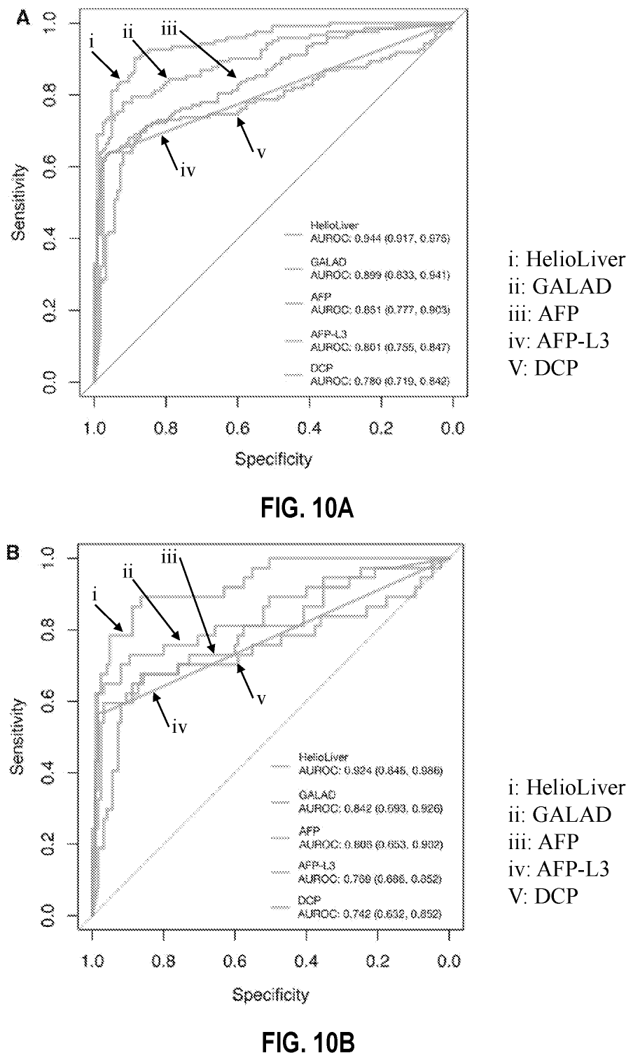

As the primary endpoint of the study, AUROC curves were used to compare the performance characteristics of the HelioLiver Test to both AFP alone and the GALAD score for the detection of HCC (FIG. 4). The HelioLiver Test demonstrated a significantly higher AUROC of 0.944 (95% CI 0.917-0.975) compared with AFP (AUROC 0.851; 95% CI 0.777-0.903; p<0.0001), AFP-L3% (AUROC 0.801; 95% CI 0.755-0.847; p<0.0001), DCP (AUROC 0.780; 95% CI 0.719-0.842; p<0.0001), and the GALAD score (AUROC 0.899; 95% CI 0.833-0.941; p<0.0001) for the detection of HCC overall (FIG. 10A). The HelioLiver Test (AUROC 0.924; 95% CI 0.846-0.986) also outperformed both AFP (AUROC 0.806; 95% CI 0.653-0902; p<0.0001), AFP-L3% (AUROC 0.769; 95% CI 0.686-0.852; p<0.0001), DCP (AUROC 0.742; 95% CI 0.632-0.852; p<0.0001), and the GALAD score (AUROC 0.842; 95% CI 0.693-0.926; p=0.0003) for the detection of early-stage (AJCC stage I and II) HCC (FIG. 10B). As anticipated, the performance of GALAD was superior to AFP, AFP-L3%, and DCP alone for detection of both HCC overall and early HCC (FIGS. 10A and 10B). To investigate whether confounding variables influenced the HelioLiver Test results, we used logistic regression analysis to assess the relationship between the patient group (subjects with HCC or control subjects) and the HelioLiver Test result in the presence of potential confounding variables including age, gender, and underlying liver disease. We then calculated an odds ratio for the HelioLiver Test result, adjusted for these potential confounders. The coefficient associated with the HelioLiver Test prediction was calculated to be 3.9 with a p value <2.2e-16 and an odds ratio=50. This suggests that patients that have a positive HelioLiver Test are approximately 50 times more likely to actually have HCC than patients with a negative test result. This odds ratio was adjusted for patient demographic data (age and gender) and the underlying liver disease of the subjects.

To further confirm that the underlying etiology of liver disease for ENCORE subjects did not influence the performance characteristics of the HelioLiver Test, a subset of 100 subjects diagnosed with HCC and 100 control subjects with matched liver disease etiologies was identified. Within the etiology-matched subgroup of subjects, the HelioLiver Test demonstrated superior performance characteristics for HCC overall (AUROC 0.933; 95% CI 0.905-0.964) compared with AFP (AUROC 0.844; 95% CI 0.789-0.898), AFP-L3% (AUROC 0.797; 95% CI 0.745-0.848; p<0.0001), DCP (AUROC 0.750; 95% CI 0.678-0.821; p<0.0001), and the GALAD score (AUROC 0.881; 95% CI 0.832-0.930). The HelioLiver Test (AUROC 0.917; 95% CI 0.866-0.968) similarly outperformed the AFP (AUROC 0.803; 95% CI 0.708-0.898), AFP-L3% (AUROC 0.765; 95% CI 0.682-0.849; p<0.0001), DCP (AUROC 0.733; 95% CI 0.622-0.844; p<0.0001), and GALAD score (AUROC 0.834; 95% CI 0.743-0.924) for the detection of early (Stage I and II) HCC within the etiology matched subgroup of subjects. As co-secondary endpoints, the sensitivity and specificity of the HelioLiver Test (using a prespecified diagnostic algorithm and cutoffs) were compared with GALAD and the individual protein tumor markers at standard clinical cutoffs. The HelioLiver Test (85.2%; 95% CI 77.8%-⁹0.4%) demonstrated a superior overall sensitivity for the detection of all-stage HCC compared with AFP at both the commonly used cutoff 20 ng/mL (62.3%; 95% CI 53.5%-70.4%) and a lower cutoff of 10 ng/mL (68.0%; 95% CI 59.3%-75.6%).

The HelioLiver Test was also more sensitive than the GALAD score at an established cutoff of −0.63 (75.4%: 95% CI 67.1%-82.2%). The HelioLiver Test demonstrated a superior sensitivity (75.7%; 95% CI 59.9%-86.7%) for early-stage (I and II) HCC when compared with AFP at both the 20-ng/mL cutoff (56.8%; 95% CI 40.1%-71.4%) and the 10-ng/mL cutoff (62.2%; 95% CI 46.1%-76.0%), and the GALAD score (64.9%; 95% CI 48.8%-78.2%) at the cutoff of −0.63. The specificity of the HelioLiver Test (91.2%; 95% CI 84.9%-95.0%) was comparable to AFP at the 10-ng/mL cutoff (90.4%; 95% CI 84.0-94.4%) and the GALAD score (93.6%; 95% CI 87.9%-96.7%). The sensitivity of both the HelioLiver Test and the GALAD score (at both the −0.63 and −1.2 cutoffs) was found to be superior to AFP-L3% (≥10% cutoff), DCP (≥7.5 ng/mL cutoff), and the combination of AFP (≥20 ng/mL cutoff) and DCP (≥7.5 ng/mL cutoff) for the detection of both HCC overall and early-stage HCC.

As an exploratory endpoint, the sensitivity of the HelioLiver Test was compared with AFP and the GALAD score at the specificity determined for the HelioLiver Test (91.2%). At this standardized specificity, the sensitivity of the HelioLiver Test for HCC detection overall was 85.2% (95% CI 77.8%-90.4%), which was higher than AFP (cutoff=12.1 ng/mL; 66.4%; 95% CI 57.6%-74.2%) and the GALAD score (cutoff=−1.2; 77.9%; 95% CI 69.8%-84.4%). The sensitivity of early-stage HCC detection for the HelioLiver Test was 75.7% (95% CI 59.9%-86.7%), which remained higher than AFP (cutoff=12.1 ng/mL; 59.5%; 95% CI 43.5%-73.4%) and the GALAD score (cutoff=−1.2; 70.3%; 95% CI 54.3%-82.5%). The sensitivity of the HelioLiver Test also remained higher than both AFP and the GALAD score at the remaining standardized specificities between 85% and 95%. The major underlying liver disease etiology in the ENCORE study was HBV, which is more prevalent in China compared with many other areas of the world. To gain insight surrounding this issue, a post hoc exploratory subgroup analysis was performed in subjects with non-HBV etiologies. AUROC of the HelioLiver Test (0.93; 95% CI 0.863-0.983) remained higher than AFP (0.913; 95% CI 0.852-0.974) and the GALAD score (0.901; 95% CI 0.825-0.977) within this non-HBV subgroup. Additionally, the sensitivity of the HelioLiver Test (86.7%; 95% CI 70.4%-94.7%) was also higher than AFP (66.7%; 95% CI 48.8%-80.8%) and the GALAD score (80.0%; 95% CI 62.7%-90.5%) within this subgroup for all HCC. These results suggest that the performance of the HelioLiver Test is etiology agnostic.

The HelioLiver Test was found to have a superior sensitivity for HCC and a similar specificity when compared to both AFP alone and the GALAD score. Most importantly, the HelioLiver Test demonstrated a superior sensitivity for early-stage (AJCC I and II) HCC when compared with either AFP testing alone or the GALAD score. The implementation of a blood test such as the HelioLiver Test will enable easy, flexible, noninvasive, and accurate HCC detection at early stages, and significantly improve treatment outcomes for a transformative reduction of HCC mortality. These findings represent a significant advancement in the field of liver cancer testing.

What is claimed is:

1. A method of generating a methylation profile of a biomarker in a subject in need thereof, comprising:
a) processing an extracted genomic DNA with a deaminating agent to generate a genomic DNA sample comprising deaminated nucleotides, wherein the extracted genomic DNA is obtained from a biological sample from the subject;
b) determining the methylation level of a CpG site of one or more genes from a group of genes comprising UBE4B, TNFAIP8L2, RASSF5, RPS6KA1, IFITM1, PPFIA1, SYT9, EVL, C16orf54, VMP1, OGFOD3, PSD4, KIAA0930, BDH1, F12, H4C6, LOC100287329, FOXP4, PTPRN2, YZ2, and LAT2 from the biological sample of the subject;
c) detecting the methylation pattern of one or more biomarkers selected from Tables 1, 2, and 6;
d) measuring methylation level of a corresponding set of genes in control samples without HCC; and
e) determining that the individual has HCC when the methylation level measured in the one or more genes is different (e.g., higher or lower) than the methylation level measured in the respective control samples.
f) determining the level of protein of one or more proteins from a group of proteins comprising AFP, AFP-L3, and DCP from the biological sample of the subject;
g) detecting a hybridization between the extracted genomic DNA and a probe, wherein the probe hybridizes to a selected region;
h) hybridizing to said one or more DNA molecules, one or more target specific DNA hybridization probes, thereby forming one or more DNA hybrids;
i) capturing the DNA hybrids with one or more bisulfite or enzymatically converted genomic DNA;
j) isolating the one or more targeted DNA hybrids;
k) amplifying the one or more captured DNA molecules if necessary;
l) sequencing the DNA molecules or the amplification products, wherein the sequencing is preferably done by means of next generation sequencing;
m) detecting the presence or absence of the cancer, with elevated level of proteins or methylation levels in the one or more genes of the individual as compared to the level of proteins or methylation in the one or more genes in the one or more control samples indicating the presence of the cancer, and the absence of elevated level of proteins or methylation levels indicating the absence of the cancer, wherein the biological sample comprises tissue or body fluid selected from the group consisting of serum, plasma, and urine.
n) determining DNA methylation status of a multitude of independent genomic CpG positions in the genome of said tumor-sample, and classifying the tumor species of the tumor-sample based on the methylation levels by using a classification-rule, wherein the classification-rule is obtained by random forest analysis of a training-data-set, and the training-data-set comprising pre-determined methylation data derived from multitude of pre-classified tumor species, wherein said pre-determined methylation data comprises the methylation status of said CpG positions in the genome of each of said pre-classified tumor species.
o) generating a prediction score based on an optimized algorithm.

2. The method of claim 1, wherein the machine learning system further incorporates imaging data, protein data, age, gender, demographic information, mutation data from specific genes and methylation profiles from the patient into the generation of the risk score.

3. The method of claim 1, wherein the generating further comprises generating a pair-wise methylation difference dataset comprising:
(i) a first difference between the methylation profile of the treated genomic DNA with a methylation profile of a first normal sample;
(ii) a second difference between a methylation profile of a second normal sample and a methylation profile of a third normal sample; and
(iii) a third difference between a methylation profile of a first primary cancer sample and a methylation profile of a second primary cancer sample.

4. In one embodiment, methods are provided for the use of artificial intelligence/machine learning systems that can incorporate and analyze structured and preferably also unstructured data to perform a risk analysis to determine a likelihood for having cancer, initially liver cancer, but also, other types of cancer, including pan-cancer testing (i.e. testing of multiple tumors from a single patient sample). By utilizing algorithms generated from the biomarker levels (e.g. methylation level or protein level or both) from large volumes of longitudinal or prospectively collected blood samples (e.g., real world data from one or more regions where blood based tumor biomarker cancer screening is commonplace) together with one or more clinical parameters (e.g. age, gender, smoking history, underlying disease signs or symptoms) a risk level or percentage of that patient having a cancer type is provided. The machine learning system determines a quantifiable risk for the presence of cancer in patients, preferably before they have symptoms or advanced disease, in terms of an increase over the population (e.g., a cohort population). By determining an individual patient's risk relative to the cohort, physicians may recommend further follow-up testing (e.g. radiography) for those patients who are at higher risks relative to the cohort population and also hope to change patient's behavior which may be increasing the risk of cancer.

5. The method of claim 3, wherein the generating further comprises analyzing the pair-wise methylation difference dataset with a control by a machine learning method to generate the methylation profile.

6. The method of claim 3, wherein the first primary cancer sample is a liver cancer sample.

7. The method of claim 3, wherein the second primary cancer sample is a non-liver cancer sample.

8. The method of claim 5, wherein the control comprises a set of methylation profiles, wherein each said methylation profile is generated from a biological sample obtained from a known cancer type.

9. The method of claim 8, wherein the known cancer type is liver cancer.

10. The method of claim 8, wherein the known cancer type is a relapsed or refractory liver cancer.

11. The method of claim 8, wherein the known cancer type is a metastatic liver cancer.

12. The method of claim 8, where the known cancer type is hepatocellular carcinoma (HCC), fibrolamellar HCC, cholangiocarcinoma, angiosarcoma, or hepatoblastoma.

13. The method of claim 4, wherein the machine learning method utilizes an algorithm selected from one or more of the following: a principal component analysis, a logistic regression analysis, a nearest neighbor analysis, a support vector machine, and a neural network model.

14. The method of claim 1, wherein the method further comprises performing a DNA sequencing reaction to quantify the methylation of each of the one or more biomarkers prior to generating the methylation profile.

15. A method of selecting a subject suspected of having liver cancer for treatment, the method comprising:
a) processing an extracted genomic DNA with a deaminating agent to generate a genomic DNA sample comprising deaminated nucleotides, wherein the extracted genomic DNA is obtained from a biological sample from the subject suspected of having liver cancer;
b) generating a methylation profile comprising one or more biomarkers selected from the Table 1, 2 and 6;
c) comparing the methylation profile of the one or more biomarkers with a control;
d) identifying the subject as having liver cancer if the methylation profile correlates to the control; and
e) administering an effective amount of a therapeutic agent to the subject if the subject is identified as having liver cancer.

16. A method of determining the prognosis of a subject having liver cancer or monitoring the progression of liver cancer in the subject, comprising:
a) processing an extracted genomic DNA with a deaminating agent to generate a genomic DNA sample comprising deaminated nucleotides, wherein the extracted genomic DNA is obtained from a biological sample from the subject having liver cancer;
b) generating a methylation profile comprising one or more biomarkers selected from the Table 2;
c) obtaining a methylation score based on the methylation profile of the one or more biomarkers; and
d) based on the methylation score, initiate a first treatment, decrease a dosage of a first therapeutic agent if the subject has experienced a remission, initiate a second treatment if the subject has experienced a relapse, or switch to a second therapeutic agent if the subject becomes refractory to the first therapeutic agent.

17. The method of any one of the claims 1-16, wherein the biological sample comprises a blood sample.

18. The method of any one of the claims 1-17, wherein the biological sample comprises a tissue biopsy sample.

19. The method of any one of the claims 1-17, wherein the biological sample comprises circulating tumor cells.

20. A method of generating a biomarker profile from a sample obtained from an individual,
wherein the biomarker profile comprises a methylation profile comprising data of one or more CpG sites from Table 11,
the method comprising:
(a) determining a methylation status for each of the one or more CpG sites of the methylation profile from a treated genomic DNA derived from the sample; and
(b) generating the methylation profile based on the methylation status of the one or more CpG site of the methylation profile to generate the biomarker profile.

21. The method of claim 20, wherein the one or more CpG sites of the methylation profile comprises one or more CpG sites of one or more of the following genes: PSD4, EVL, RASSF5, MAP3K8, LAT2, HEXDC, MYO1G, CTTN, UBE4B, KIAA0930, LTA, C16orf54, LOC101928253, URI1, TNFAIP8L2 (SCNM1), FOXP4 (AS1), IFITM1, RPS6KA1, LINC01298, HIST1H4F, BDH1, MIR153-2, PFN3, LOC101929153, MIR1302-7, LOC100506585, DIRAS1, or MIR21.

22. The method of claim 20 or 21, wherein the one or more CpG sites of the methylation profile comprises one or more CpG sites of the following genes: PSD4, EVL, RASSF5, MAP3K8, LAT2, HEXDC, MYO1G, CTTN, UBE4B, KIAA0930, LTA, C16orf54, LOC101928253, URI1, TNFAIP8L2 (SCNM1), FOXP4 (AS1), IFITM1, RPS6KA1, LINC01298, HIST1H4F, BDH1, MIR153-2, PFN3, LOC101929153, MIR1302-7, LOC100506585, DIRAS1, and MIR21.

23. The method of claims 20 or 21, wherein the one or more CpG sites of the methylation profile comprises one or more of the following CpG sites: chr17:57915773-57915774, chr19:2723147-2723148, chr19:2723034-2723035, chr17:57915717-57915718, chr5:4629212-4629213, chr5:4629193-4629194, chr19:2723181-2723182, chr19:2723169-2723170, chr6:26240930-26240931, chr6:26240920-26240921, chr19:30562385-30562386, chr19:30562320-30562321, chr11:314074-314075, chr6:26240975-26240976, chr6:26240950-26240951, chr6:26240939-26240940, chr19:2723189-2723190, or chr19:2723184-2723185.

24. The method of claims 20 or 21, wherein the one or more CpG sites of the methylation profile comprises the following CpG sites: chr17:57915773-57915774, chr19:2723147-2723148, chr19:2723034-2723035, chr17:57915717-57915718, chr5:4629212-4629213, chr5:4629193-4629194, chr19:2723181-2723182, chr19:2723169-2723170, chr6:26240930-26240931, chr6:26240920-26240921, chr19:30562385-30562386, chr19:30562320-30562321, chr11:314074-314075, chr6:26240975-26240976, chr6:26240950-26240951, chr6:26240939-26240940, chr19:2723189-2723190, and chr19:2723184-2723185.

25. The method of any one of claims 20-24, wherein the one or more CpG sites of the methylation profile comprises the following CpG sites: chr17:57915773-57915774, chr19:2723147-2723148, chr19:2723034-2723035, chr17:57915717-57915718, chr5:4629212-4629213, chr5:4629193-4629194, chr19:2723181-2723182, chr19:2723169-2723170, chr6:26240930-26240931, chr6:26240920-26240921, chr19:30562385-30562386, chr19:30562320-30562321, chr11:314074-314075, chr6:26240975-26240976, chr6:26240950-26240951, chr6:26240939-26240940, chr19:2723189-2723190, chr19:2723184-2723185, chr8:142852883-142852884, chr8:142852876-142852877, chr7:157563602-157563603, chr11:314113-314114, chr11:314106-314107, chr11:314098-314099, chr11:314086-314087, chr1:206753453-206753454, chr7:157319206-157319207, chr7:157319203-157319204, chr7:157319199-157319200, chr1:151129298-151129299, chr7:73641105-73641106, chr7:73641071-73641072, chr16:29757375-29757376, chr16:29757360-29757361, chr11:70211540-70211541, chr11:70211534-70211535, chr11:70211531-70211532, chr11:70211523-70211524, chr14:100532797-100532798, chr14:100532790-100532791, chr5:176829777-176829778, chr5:176829755-176829756, chr16:29757350-29757351, chr16:29757323-29757324, chr3:197283111-197283112, chr6:11976066-11976067, chr6:11976024-11976025, chr6:41528502-41528503, chr6:41528499-41528500, chr6:41528497-41528498, chr6:41528491-41528492, chr16:29757344-29757345, chr16:29757334-29757335, chr17:80358932-80358933, chr17:80358919-80358920, chr6:31527920-31527921, chr6:31527893-31527894, chr6:31527889-31527890, chr2:113931525-113931526, chr2:113931518-113931519, chr7:45018849-45018850, chr8:96193941-96193942, chr8:96193898-96193899, chr1:26872538-26872539, chr1:26872525-26872526, chr1:26872518-26872519, chr22:45631384-45631385, chr22:45631379-45631380, chr10:30818618-30818619, chr10:30818611-30818612, chr10:30818609-30818610, chr1:10134620-10134621, chr1:10134610-10134611, chr17:80358850-80358851, chr17:80358847-80358848, chr17:80358829-80358830, and chr17:80358819-80358820.

26. The method of any one of claims 20-25, wherein the methylation status of each CpG site is based on a p-value, and wherein the 0-value of a CpG site is determined based on the proportion of instances of methylation at the CpG site divided by the sum of the instances of methylation at the CpG site plus the instances where the CpG site is not methylated.

27. The method of any one of claims 20-26, wherein the methylation status is determined using sequencing information derived from the treated genomic DNA.

28. The method of claim 27, wherein the sequencing information is obtained using a sequencing technique.

29. The method of claim 28, wherein the sequencing technique is a next generation sequencing technique.

30. The method of claim 28 or 29, wherein the sequencing technique is a whole-genome sequencing technique.

31. The method of claim 28 or 29, wherein the sequencing technique is a targeted sequencing technique.

32. The method of any one of claims 28-31, wherein the sequence technique is capable of providing paired-end sequencing reads.

33. The method of any one of claims 28-32, wherein the sequencing technique is performed such that the sequencing depth is at least about 50×.

34. The method of any one of claims 28-33, further comprising performing the sequencing technique.

35. The method of any one of claims 20-34, further comprising obtaining the treated genomic DNA derived from the sample.

36. The method of claim 35, wherein the obtaining the treated genomic DNA comprises subjecting DNA derived from the sample to processing that enables determination of a methylation status of a CpG.

37. The method of claim 36, wherein the processing to obtain the treated genomic DNA comprises an enzyme-based technique for the conversion of unmethylated cytosines to enable the determination of the methylation status of a CpG site.

38. The method of claim 37, wherein the enzyme-based technique is an EM-seq technique.

39. The method of claim 36, wherein the processing to obtain the treated genomic DNA comprises a bisulfite-based technique.

40. The method of any one of claims 20-39, wherein the detecting the methylation status for each of the one or more CpG sites is based on sequence reads obtained from the treated genomic DNA.

41. The method of claim 40, wherein the sequence reads used for the detecting the methylation status for each of the one or more CpG sites are pre-processed.

42. The method of claim 41, wherein the sequence read pre-processing comprises removing low-quality reads.

43. The method of claim 41 or 42, wherein the sequence read pre-processing comprises removing sequence adaptor sequences.

44. The method of any one of claims 41-43, wherein the sequence read pre-processing comprises removing M-bias.

45. The method of any one of claims 41-44, wherein the sequence read pre-processing comprises producing paired reads.

46. The method of any one of claims 41-45, wherein the sequence read pre-processing comprises removing sequence reads having a sequencing depth of less than 50×.

47. The method of any one of claims 41-46, wherein the sequence read pre-processing comprises mapping sequence reads to a reference genome.

48. The method of claim 47, wherein the reference genome is a human reference genome.

49. The method of any one of claims 20-48, wherein the biomarker profile further comprises a polypeptide profile.

50. The method of claim 49, wherein the polypeptide profile comprises data of one or more of an alpha fetoprotein (AFP) level, a Lens culinaris agglutinin-reactive AFP (AFP-L3%) level, or a des-gamma-carboxyprothrombin (DCP) level obtained from the individual.

51. The method of claim 50, wherein the polypeptide profile comprises data of the AFP level, AFP-L3%, and the DCP level.

52. The method of claim 50 or 51, wherein the AFP level, AFP-L3%, and DCP level are based on respective serum concentrations measured from the individual.

53. The method of claim 52, wherein the serum concentrations are derived from the sample obtained from the individual.

54. The method of any one of claims 20-53, wherein the biomarker profile further comprises a demographic profile.

55. The method of claim 54, wherein the demographic profile comprises the age of the individual.

56. The method of claim 55, wherein the demographic profile comprises the sex of the individual.

57. A method of generating a biomarker profile from a sample obtained from an individual,
wherein the biomarker profile comprises:
a methylation profile comprising data of one or more CpG sites from Table 11;
a polypeptide profile comprising data of one or more of an AFP level, an AFP-L3%, or a DCP level; and
a demographic profile comprising data of one or more of the age or sex of the individual,
the method comprising:
(a) determining, for the methylation profile, a methylation status for each of the one or more CpG sites of the methylation profile from a treated genomic DNA derived from the sample;
(b) determining, for the polypeptide profile, one or more the AFP level, the AFP-L3%, or the DCP level from the sample;
(c) determining, for the demographic profile, one or more of the age or sex of the individual; and
(d) generating the biomarker profile based on the methylation profile, the polypeptide profile, and the demographic profile.

58. The method of claim 57, wherein the methylation profile comprises data of all CpG sites from Table 11.

59. The method of claim 57 or 58, wherein the polypeptide profile comprises the AFP level, the AFP-L3%, and the DCP level.

60. The method of any one of claims 57-59, wherein the demographic profile comprises the age and sex of the individual.

61. The method of any one claims 20-60, wherein the generating the biomarker profile comprises providing the methylation profile, the polypeptide profile, and/or the demographic profile to one or more machine learning classifiers to generate the biomarker profile.

62. The method of claim 61, wherein the one or more machine learning classifiers comprises a random forest model.

63. The method of claim 61 or 62, wherein the one or more machine learning classifiers comprises a grid-search technique.

64. The method of claim 63, wherein the grid-search technique comprises optimizing the hyper parameters of the random forest model.

65. The method of any one of claims 61-64, wherein the biomarker profile combines the methylation profile, the polypeptide profile, and/or the demographic profile using a decision tree model.

66. The method of any one of claims 61-65, wherein at least one of the one or more machine learning classifiers is trained using a data derived from one or more individuals having known condition(s) and one or more associated methylation profiles, polypeptide profiles, or demographic profiles.

67. The method of claim 66, wherein the known condition is whether the individual has a liver cancer or chronic liver disease.

68. The method of any one of claims 20-67, wherein the sample is a liquid biopsy sample.

69. The method of any one of claims 20-68, wherein the sample is a blood sample.

70. The method of any one of claims 20-69, wherein the sample comprises cfDNA.

71. The method of any one of claims 20-70, wherein the sample is a cfDNA sample.

72. The method of any one of claims 20-71, wherein the subject is suspected of having a liver cancer.

73. The method of any one of claims 20-72, wherein the liver cancer is hepatocellular carcinoma.

74. A system for determining a biomarker profile from a sample obtained from an individual,
wherein the biomarker profile comprises one or more of:
a methylation profile comprising data of one or more CpG sites from Table 11;
a polypeptide profile comprising data of one or more of an AFP level, an AFP-L3%, or a DCP level; or
a demographic profile comprising data of one or more of the age or sex of the individual,
the system comprising:
one or more processors; and
memory storing one or more programs, the one or more programs configured to be executed by the one or more processors, the one or more programs including instructions for:
receiving sequencing information comprising sequence reads;
determining one or more of the following:
the methylation profile based on data of the one or more CpG sites from Table 11;
the polypeptide profile based on data of the one or more of the AFP level, the AFP-L3%, or the DCP level; or
the demographic profile based on data of the one or more of the age or sex of the individual,
determining the biomarker profile based on one or more of the methylation profile, the polypeptide profile, or the demographic profile.

75. The system of claim 74, further comprising one or more machine learning classifiers configured to determine the biomarker profile.

76. A system for determining a biomarker profile from a sample obtained from an individual,
wherein the biomarker profile comprises one or more of:
a methylation profile comprising data of one or more CpG sites from Table 11;
a polypeptide profile comprising data of one or more of an AFP level, an AFP-L3%, or a DCP level; or
a demographic profile comprising data of one or more of the age or sex of the individual,
the system comprising:
one or more processors; and
memory storing one or more programs, the one or more programs configured to be executed by the one or more processors, the one or more programs including instructions for:
receiving data pertaining to one or more of the methylation profile, the polypeptide profile, and the demographic profile;
applying one or more machine learning classifiers to the received data to determine the biomarker profile based on one or more of the methylation profile, the polypeptide profile, or the demographic profile.

77. The system of claim 76, wherein the one or more machine learning classifiers comprises a random forest model.

78. The system of claim 76 or 77, wherein the one or more machine learning classifiers comprises a grid-search technique.

79. The system of claim 78, wherein the grid-search technique comprises optimizing the hyper parameters of the random forest model.

80. The system of any one of claims 76-79, wherein the biomarker profile combines the methylation profile, the polypeptide profile, and/or the demographic profile using a decision tree model.

81. The system of any one of claims 76-80, wherein at least one of the one or more machine learning classifiers is trained using a data derived from one or more individuals having known condition(s) and one or more associated methylation profiles, polypeptide profiles, or demographic profiles.

82. The system of claim 81, wherein the known condition is whether the individual has a liver cancer or chronic liver disease.

83. A kit for generating a biomarker profile from a sample from an individual, the kit comprising one or more probes, wherein each probe is suitable for detecting a methylation status of a CpG site in Table 11.

84. The kit of claim 83, wherein each probe hybridizes to at least a portion of the targeted region in Table 11.

85. The kit of claim 84, wherein the at least the portion is at least about 50 base pairs.

86. The kit of claim 85, wherein the at least the portion is about 120 base pairs.

87. The kit of 85 or 86, wherein the each probe is complementary to the target portion.

88. The kit of any one of claims 83-87, wherein each probe is about 50 to about 120 base pairs.

89. The kit of any one of claims 83-88, wherein each probe is configured to determine the methylation status of one or more CpG sties from Table 11.

90. The kit of any one of claims 83-89, further comprising reagents to determine one or more of an AFP level, an AFP-L3%, or a DCP level from a sample from the individual.

91. The kit of any one of claims, further comprising instructions for determining the age and/or sex of the individual.
